(19) United States
(12) Patent Application Publication
(10) Pub. No.: US 2009/0044291 A1
(43) Pub. Date: Feb. 12, 2009

(54) DROUGHT-RESISTANT PLANTS AND METHOD FOR PRODUCING THE PLANTS

(75) Inventors: Da-Peng Zhang, Beijing (CN); Yuan-Yue Shen, (US); Xiao-Fang Wang, Beijing (CN); Fu-Qing Wu, (US); Shu-Yuan Du, (US); Zheng Cao, (US); Yi Shang, (US); Yan-Hong Xu, Beijing (CN)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: D-Helix, San Francisco, CA (US)

(21) Appl. No.: 11/846,471

(22) Filed: Aug. 28, 2007

Related U.S. Application Data
(60) Provisional application No. 60/841,505, filed on Aug. 31, 2006.

(51) Int. Cl. C12N 15/54; A01H 5/00

(52) U.S. Cl. 800/278; 536/23.2; 800/298

(57) ABSTRACT

This application provides a recombinant expression cassette for expressing the H subunit of Mg-chelatase, a plant gene product that is newly identified as an abscisic acid receptor. Also provided are a transgenic plant with drought-resistance and a method for producing such plants.

### CROSS-REFERENCES TO RELATED APPLICATIONS

The present patent application claims benefit of priority to U.S. Provisional Patent Application No. 60/841,505, filed Aug. 31, 2006, the contents of which are incorporated by reference in the entirety for all purposes.

### FIELD OF THE INVENTION

This invention relates to methods and compositions for generating plants with altered abscisic acid sensitivity.

### BACKGROUND OF THE INVENTION

The phytohormone abscisic acid (ABA) regulates many agriculturally important stress and developmental responses throughout the life cycle of plants. In seeds, ABA is responsible for the acquisition of nutritive reserves, desiccation tolerance, maturation and dormancy (M. Koornneef et al., Plant Physiol. Biochem., 36:83 (1998); J. Leung & J. Giraudat, Annu. Rev. Plant. Physiol. Plant. Mol. Biol., 49:199 (1998)). During vegetative growth, ABA is a central internal signal that triggers plant responses to various adverse environmental conditions including drought, salt stress and cold (M. Koornneef et al., Plant Physiol. Biochem., 36:83 (1998); J. Leung & J. Giraudat, Annu. Rev. Plant. Physiol. Plant. Mol. Biol., 49:199 (1998)). A rapid response mediated by ABA is stomatal closure in response to drought (J. Leung & J. Giraudat, Annu. Rev. Plant. Physiol. Plant. Mol. Biol., 49:199 (1998); E. A. C. MacRobbie, Philos. Trans. R Soc. Lond. B Biol. Sci., 353:1475 (1998); J. M. Ward et al., Plant Cell, 7:833 (1995)). Stomata on the leaf surface are formed by pairs of guard cells whose turgor regulates stomatal pore apertures (E. A. C. MacRobbie, Philos. Trans. R Soc. Lond. B Biol. Sci., 353:1475 (1998); J. M. Ward et al., Plant Cell, 7:833 (1995)). ABA induces stomatal closure by triggering cytosolic calcium ([Ca²⁺_{cyt}) increases which regulate ion channels in guard cells (E. A. C. MacRobbie, Philos. Trans. R Soc. Lond. B Biol. Sci., 353:1475 (1998); J. M. Ward et al., Plant Cell, 7:833 (1995)). This response is vital for plants to limit transpirational water loss during periods of drought.

### BRIEF SUMMARY OF THE INVENTION

The present inventors have discovered, for the first time, that the H subunit of Mg-chelatase (CHLH) is a receptor for abscisic acid (ABA), a phytohormone that plays a role in plant physiology. Because ABA is involved in regulating the opening of stomatal aperture, an important mechanism for a plant to adjust transpirational water loss in response to changes in water availability in the environment, this discovery provides a method for increasing drought tolerance in a plant, as well as other stress tolerance related to ABA-sensitivity. This method involves expressing CHLH (also referred to herein as the “ABA receptor” or “ABAR”), in a plant, for instance, by introducing a recombinant expression vector comprising a heterologous promoter and a CHLH-coding sequence into the plant. The heterologous promoter and the CHLH-coding sequence being operably linked in the expression vector, CHLH is therefore expressed in the plant and confers enhanced ABA sensitivity to the plant.

The present invention provides methods of enhancing abscisic acid sensitivity in a plant. In some embodiments, the methods comprise introducing an recombinant expression cassette into a plant, wherein the expression cassette comprises a promoter operably linked to a polynucleotide encoding the H subunit of Mg-chelatase, wherein the promoter is heterologous to the polynucleotide, wherein the plant has increased abscisic acid sensitivity compared to an otherwise identical plant lacking the expression cassette.

In some embodiments, the plant has improved drought tolerance compared to an otherwise identical plant lacking the expression cassette

In some embodiments, the H subunit of Mg-chelatase is at least 80% identical to a protein selected from the group consisting of SEQ ID NO:2, SEQ ID NO:4, SEQ ID NO:6, SEQ ID NO:8, SEQ ID NO:10 and SEQ ID NO:12.

In some embodiments, the promoter is constitutive. In some embodiments, the promoter is inducible. In some embodiments, the promoter is tissue-specific. In some embodiments, the promoter directs expression in guard cells, for example is guard cell specific.

In some embodiments, the methods comprise generating a plurality of plants comprising the introduced expression cassette, and screening the plants for abscisic acid sensitivity compared to an otherwise identical plant lacking the expression cassette.

The present invention also provides methods of decreasing abscisic acid sensitivity in a plant. In some embodiments, the methods comprise introducing an recombinant expression cassette into a plant, wherein the expression cassette comprises a promoter operably linked to a polynucleotide comprising at least 20 nucleotides complementary or identical to a contiguous sequence in an mRNA encoding a H subunit of Mg-chelatase in the plant, wherein the promoter is heterologous to the polynucleotide, thereby reducing expression of the H subunit Mg-chelatase in the plant, wherein the plant has reduced abscisic acid sensitivity compared to an otherwise identical plant lacking the expression cassette.

In some embodiments, the polynucleotide comprises at least 50 nucleotides complementary or identical to a contiguous sequence in a cDNA encoding a H subunit of Mg-chelatase in the plant. In some embodiments, the polynucleotide comprises at least 200 nucleotides complementary or identical to a contiguous sequence in an cDNA encoding a H subunit of Mg-chelatase in the plant.

In some embodiments, the H subunit of Mg-chelatase is at least 80% identical to a protein selected from the group consisting of SEQ ID NO:2, SEQ ID NO:4, SEQ ID NO:6, SEQ ID NO:8, SEQ ID NO:10 and SEQ ID NO:12. In some embodiments, the polynucleotide comprises at least 20, 50, 100 or 200 nucleotides complementary or identical to a contiguous sequence of SEQ ID NO:1, SEQ ID NO:3, SEQ ID NO:5, SEQ ID NO:7, SEQ ID NO:9, or SEQ ID NO:11.

In some embodiments, the promoter directs expression of the polynucleotide to abscission zones of the plant.

The present invention also provides for recombinant expression cassettes comprising a promoter operably linked to a polynucleotide encoding the H subunit of Mg-chelatase, wherein the promoter is heterologous to the polynucleotide, and wherein introduction of the expression cassette into a plant results in enhanced abscisic acid sensitivity in the plant compared to an otherwise identical plant lacking the expression cassette.

In some embodiments, introduction of the expression cassette into a plant results in improved drought tolerance in the plant compared to an otherwise identical plant lacking the expression cassette

In some embodiments, the H subunit of Mg-chelatase is at least 80% identical to a protein selected from the group consisting of SEQ ID NO:2, SEQ ID NO:4, SEQ ID NO:6, SEQ ID NO:8, SEQ ID NO:10 and SEQ ID NO:12.

In some embodiments, the promoter is constitutive. In some embodiments, the promoter is inducible. In some embodiments, the promoter is tissue-specific. In some embodiments, the promoter directs expression in guard cells.

The present invention also provides for transgenic plants comprising a recombinant expression cassette, wherein the expression cassette comprises a promoter operably linked to a polynucleotide encoding the H subunit of Mg-chelatase, wherein the promoter is heterologous to the polynucleotide, and wherein the plant has enhanced abscisic acid sensitivity compared to an otherwise identical plant lacking the expression cassette.

In some embodiments, the plant has improved drought tolerance compared to an otherwise identical plant lacking the expression cassette.

In some embodiments, the H subunit of Mg-chelatase is at least 80% identical to a protein selected from the group consisting of SEQ ID NO:2, SEQ ID NO:4, SEQ ID NO:6, SEQ ID NO:8, SEQ ID NO:10 and SEQ ID NO:12.

In some embodiments, wherein the promoter is constitutive. In some embodiments, the promoter is inducible. In some embodiments, the promoter is tissue-specific. In some embodiments, the promoter directs expression in guard cells.

The invention also provides for any plant part from the transgenic plants of the invention. Examples of such plant parts include, but are not limited to: seeds, flowers, leafs and fruits.

### DEFINITIONS

“The H subunit of Mg-chelatase” refers to the H subunit of Mg-chelatase. Mg-chelatase is a multi-subunit enzyme that catalyzes Mg²⁺ into a protoporphyrin in the chlorophyll synthesis pathway. See, e.g., Walker et al., Biochem. J. 327-321-333 (1997), incorporated herein by reference. Exemplary H subunits of Mg chelatase are encompassed by the consensus sequence provided in FIG. 1 of Walker et al., Biochem. J. 327-321-333 (1997). The H subunit of Mg-chelatase can be from photosynthetic bacteria or plants. In some embodiments, the H subunit of Mg-chelatase is substantially identical to any of SEQ ID NO:2, SEQ ID NO:4, SEQ ID NO:6, SEQ ID NO:8, SEQ ID NO:10 and SEQ ID NO:12.

The term “nucleic acid” or “polynucleotide” refers to a single or double-stranded polymer of deoxyribonucleotide or ribonucleotide bases read from the 5′ to the 3′ end, or an analog thereof.

The term “promoter,” as used herein, refers to a polynucleotide sequence capable of driving transcription of a coding sequence in a cell. Thus, promoters used in the polynucleotide constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “plant promoter” is a promoter capable of initiating transcription in plant cells. A “constitutive promoter” is one that is capable of initiating transcription in nearly all tissue types, whereas a “tissue-specific promoter” initiates transcription only in one or a few particular tissue types.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid, and hemizygous.

A polynucleotide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., is a genetically engineered coding sequence, e.g., from a different gene in the same species, or an allele from a different ecotype or variety).

A polynucleotide “exogenous” to an individual plant is a polynucleotide which is introduced into the plant by any means other than by a sexual cross. Examples of means by which this can be accomplished are described below, and include Agrobacterium-mediated transformation, biolistic methods, electroporation, and the like. Such a plant containing the exogenous nucleic acid is referred to here as a T₁ (e.g., in Arabidopsis by vacuum infiltration) or R₀ (for plants regenerated from transformed cells in vitro) generation transgenic plant.

As used herein, the term “transgenic” describes a non-naturally occurring plant that contains a genome modified by man, wherein the plant includes in its genome an exogenous nucleic acid molecule, which can be derived from the same or a different plant species. The exogenous nucleic acid molecule can be a gene regulatory element such as a promoter, enhancer, or other regulatory element, or can contain a coding sequence, which can be linked to a heterologous gene regulatory element. Transgenic plants that arise from sexual cross or by selfing are descendants of such a plant.

An “expression cassette” refers to a nucleic acid construct that, when introduced into a host cell, results in transcription and/or translation of an RNA or polypeptide, respectively. Antisense or sense constructs that are not or cannot be translated are expressly included by this definition. In the case of both expression of transgenes and suppression of endogenous genes (e.g., by antisense, or sense suppression) one of skill will recognize that the inserted polynucleotide sequence need not be identical, but may be only “substantially identical” to a sequence of the gene from which it was derived. As explained below, these substantially identical variants are specifically covered by reference to a specific nucleic acid sequence.

“Increased” or “enhanced” CHLH expression or activity refers to an augmented change in the protein's expression or activity. Examples of such increased activity or expression include the following: CHLH expression or activity is increased above the level of that in wild-type, non-transgenic control plants (i.e., the quantity of CHLH activity or expression of the CHLH gene is increased). CHLH expression or activity is present in an organ, tissue, or cell where it is not normally detected in wild-type, non-transgenic control plants (i.e., CHLH expression or activity is increased within certain tissue types). CHLH expression or activity is increased when its expression or activity is present in an organ, tissue or cell for a longer period than in a wild-type, non-transgenic controls (i.e., duration of CHLH expression or activity is increased).

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

The phrase “substantially identical,” used in the context of two nucleic acids or polypeptides, refers to a sequence that has at least 25% sequence identity with a reference sequence. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 90%, 95%, or 99%, compared to a reference sequence using the programs described herein; preferably BLAST using standard parameters, as described below. This definition also refers to the complement of a test sequence, when the test sequence has substantial identity to a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

(see, e.g., Creighton, Proteins (1984)).

As used herein, the term “drought-resistance” or “drought-tolerance,” including any of their variations, refers to the ability of a plant to recover from periods of drought stress (i.e., little or no water for a period of days). Typically, the drought stress will be at least 5 days and can be as long as 18 to 20 days or more, depending on, for example, the plant species.

### BRIEF DESCRIPTION OF THE DRAWINGS

FIG. 1 illustrates that ABAR binds ABA. a, Saturable ABA-specific binding (filled circles) to the pure yeast-expressed ABAR. Open circles, non-specific binding. b, Scatchard plot of binding data in a. B, [3H]ABA bound; F, free [3H]ABA. The parameters of the curve are Kd=32 nM; Bmax=1.28 mol mol−1; R2=0.98. c, Displacement of [3H](+)-ABA binding by (+)-ABA and (−)-ABA. Filled triangles, in vitro (+)-ABA binding (mol mol−1); open triangles, in vitro (−)-ABA binding (mol mol-1); filled circles, (−)-ABA pull-down assay (d.p.m.); open circles, (+)-ABA pull-down assay (d.p.m.). d, Saturable ABA-specific binding to the extracts of leaves of gl1 (WT), RNAi (line 12) and overexpressing (OE, line 1) lines. NB, non-specific binding. e, Immunosignal of ABAR protein in the extracts in d and the band intensities relative to the wild type. Each point in a-d is the mean plusminus s.d. (n=10).

FIG. 2 illustrates scatchard plot of binding data in FIG. 1d1. For the wild-type plants (WT), Kd (equilibrium dissociation constant)=35 nM and Bmax (maximum binding)=60 nmol ABA g⁻¹ crude protein (R²=0.96); for the RNAi transgenic plants (RNAi), Kd=36 nM and Bmax=25 nmol g⁻¹ (R²=0.96); and for the overexpressors of ABAR/CHLH gene (OE), Kd=38 nM and Bmax=80 nmol g⁻¹ (R²=0.95). B, [³H]ABA bound; F, [³H]ABA free.

FIG. 3 illustrates regulation of ABAR expression levels alters ABA binding activity. [³H](+)ABA radioactivity was pulled down by immunoprecipitation with anti-ABAR serum in the extracts prepared from the leaves of the WT, RNAi or OE lines (columnar panel). The amounts of the immuno-precipitated ABAR protein form the corresponding extracts, determined by immunoblotting with anti-ABAR serum, are displayed below the columnar panel. Values below the immuno-bands indicate the band intensities relative to WT. Addition of mouse preimmune serum instead of the antiserum to the medium of the pull down assay in the extracts from the leaves of wild-type plants was taken as a control (CK1). A second control experiment (CK2) was conducted with the supernatants obtained after the precipitation of the wild-type plant extracts with the preimmune serum. The third control (CK3) was done by depleting ABAR protein with the anti-ABAR serum from the wild-type plant extracts, in which the supernatants after the immuno-depletion were used to assay either [³H]ABA binding or residual ABAR protein by immunoblotting with anti-ABAR serum. The treatments with preimmune serum could neither pull down any ABA-binding activity (CK1) nor substantially affect ABA-binding activity in the wild-type plant extracts (CK2). The level of ABA-binding activity in the preimmune serum-treated extracts of wild-type plants is comparable to that of the pulled down-binding activity from the wild-type plant extracts (CK2, WT). The anti-ABAR serum depletes most of the ABA-binding activity from the wild-type plant extracts (CK3). The data from the control experiments indicate that the pull down assays are specific. d.p.m., disintegrations per minute. The RNAi line used was the line 12, and OE was the line 1 (see FIG. 5). Each value in the columnar panel is the mean±s.d. (n=5).

FIG. 4 illustrates changes in ABAR expression alter plant sensitivity to ABA. a, b, Seed germination (a) and seedling (10 days in ABA) growth (b) of gl1 (WT, white columns in a), RNAi (black columns in a) and overexpressing (OE, hatched columns in a) lines in medium containing (plusminus)-ABA. ABA concentrations are in muM. c, ABA-induced stomatal closure (top) and inhibition of opening (middle). Black columns, initial stomatal aperture; grey columns, apertures after ABA treatment. ABA concentrations are in muM. Bottom: stomatal apertures in the assay of inhibition of opening by 2 muM ABA. d, Water loss from detached leaves. Open circles, WT; filled circles, OE; triangles, RNAi. e, Plant status after drought treatment (controls were fully watered plants). The RNAi line 12 and OE line 1 were used. Error bars indicate s.d. (n=5).

FIG. 5 illustrates negative correlation of ABAR levels with ABA-insensitive phenotypes in transgenic plants. For estimating the ABA-responsive phenotype intensity of different transgenic lines in germination, seedling growth and stomatal movement, the values of wild-type (gl 1) plants obtained in the exogenously applied (±)-ABA concentrations (1, 2, and 5 μM for germination or stomatal aperture, and 1, 5, 10, and 20 μM for seedling growth, see FIG. 4) were taken as 1. The assays of the estimation were repeated thrice where each repetition in each line contained about 300 seeds for germination assay, 80 seedlings for postgermination growth and more than 30 plants for stomatal observations. The values higher than those of gl1 plants were considered to be positive and the lower to be negative. The maximum value in each class of phenotypes (germination, seedling growth or stomatal movement) was referred to as 5. The ‘phenotype intensity’ for each transgenic line was estimated by the averages based on these calculations. The amounts of ABAR protein were determined by immunoblotting of which the intensity of the resulting immuno-bands was estimated by densitometric scans using a digital imaging system. The intensity of the ABA-insensitive phenotypes in stomatal response is negatively correlated with the ABAR expression levels. A globally negative correlation of the ABA-insensitive phenotype intensity with the ABAR/CHLH levels is found in germination and postgermination growth, but the phenotypes become weaker when the ABAR/CHLH levels are low to a certain extent (indicated by arrows), suggesting that some quantitative threshold of this protein may exist in the signaling process of germination and early growth of seedlings.

FIG. 6 illustrates ABAR knockout mutation confers immature embryo. a, Position of T-DNA insertion in the 1st exon of ABAR gene at 791 bp downstream of start codon. b, A picture of embryos from the wild-type seed (WT, the ecotype Colombia) and mutant seed (abar-1) recovered heterozygous plants. c, A section of wild-type seed (transferred to light at 20° C. for 14 h after stratification) shown by light microscopy (left panel) and by confocal laser scanning microscopy with immuno-staining by anti-ABAR serum and fluorescein isothiocyanate (right panel). Bar=100 μm. d, A section of the knockout mutant seed (transferred to light at 20° C. for 72 h after stratification) observed as in c. The late- or non-germinating seeds, small in their size, were shown to be the abar-1 seeds in which the ABAR protein was not detectable by the immuno-staining with the ABAR-specific antiserum. e, Structure of a wild-type embryonic cell, showing plenty of lipid bodies and mature protein bodies in the cell. f, Structure of the mutant abar-1 embryonic cells, showing deficiency of lipid bodies and presence of immature protein bodies in these cells. Bars in e and f represent 2 μm. Abbreviations: c, cotyledon; h, hypocotyl; ipb, immature protein body; lb, lipid body; pb, protein body.

FIG. 7 illustrates ABAR-mediated ABA signaling is a distinct process. a, ABA treatment decreases the chlorophyll (filled triangles) and Proto (open triangles) levels, but enhances MgProto (filled circles) and ABAR protein (diamonds) levels and Mg-chelatase (open circles) activity in gl1 seedlings. b, Oestradiol (ED)-induced downregulation of ABAR expression (top panel; immunosignal and its relative intensity) in the inducible RNAi plants results in ABA-insensitive phenotypes in both stomatal closure (middle panel) and inhibition of stomatal opening (bottom panel) induced by 10 muM ABA. c, The ABAR level (indicated by arrow and relative band intensity) is reduced in cch, but not in the other mutants containing different levels of chlorophyll. Filled columns, chlorophyll a; open columns, chlorophyll b. d-f, cch is an ABA-insensitive mutant in germination (d, 1 muM ABA), seedling growth (e, root length of 7-day-old seedlings in 10 muM ABA) and stomatal movement (f, top, stomatal closure; bottom, stomatal opening; 10 muM ABA was used). Filled columns in d and e, with ABA; open columns, without ABA. Black columns in f, with ABA, 2 h; grey columns, without ABA, 2 h; white columns, without ABA, 0 h. Error bars indicate s.d. (n=5).

FIG. 8 illustrates Protoporphyrin IX (Proto) is not able to displace [³H]ABA bound to ABAR. Proto-pp, binding assay with the pure yeast-expressed ABAR (data as mol mol⁻¹); Proto-pd, pull-down assay (data as d.p.m., disintegrations per minute). The displacement by (+)-ABA was taken as a control (ABA-pp for Proto-pp and ABA-pd for Proto-pd).

FIG. 9 illustrates the levels of ABAR/CHLH protein, but not the contents of chlorophyll (Chl) or Mg-protoporphyrin IX (MgProto), are correlated with ABA-responsive stomatal movement. The changes of Chl and MgProto (top panel) and levels of ABAR/CHLH protein (below the top panel, estimated by immunoblotting) after the treatment of gl1 plants with chloramphenical (CP) were compared with corresponding effects of ABA on promoting stomatal closure (middle columnar panel) and on inhibiting stomatal opening (bottom columnar panel). ABA contents (μg g⁻¹ dry weight) remained unchanged in either CP-non-treated ([ABA]−CP) or CP-treated ([ABA]+CP) plants during the period. In the top columnar panel, ‘Chla−CP’ or ‘MgProto−CP’ and ‘Chla+CP’ or ‘MgProto+CP’ denote, respectively, Chl a or MgProto contents in the CP-non-treated (−CP) and CP-treated (+CP) plants. Error bars in the ‘Chla+CP’ columns are for total Chl (Chl a plus chl b). In the middle columnar panel, stomatal apertures were scored before ABA treatment in the CP-non-treated (−CP) and CP-treated (+CP) plants, and they were assayed again 2 h after 10 μM (±)-ABA treatment in these −CP(−CP+ABA) and +CP (+CP+ABA) plants. In the bottom columnar panel, the stomatal initial apertures were scored in the CP-non-treated (−CP/init) and CP-treated (+CP/init) plants, and 2 h later, stomatal apertures were assayed again in the CP-non-treated plants subjected to the ABA-free-(−CP−ABA) or 10 μM (±)-ABA-treatment (−CP+ABA), and also in the CP-treated plants subjected to the ABA-free-(+CP−ABA) or 10 μM (±)-ABA-treatment (+CP+ABA). The contents of Chl (Chl a plus Chl b) and MgProto as well as stomatal apertures are presented in percentages relative to those of the CP-non-treated plants, i.e., the values of the columns ‘Chla-CP’ (Chl a plus Chl b) and ‘MgProto-CP’ are taken as 100%, respectively, for Chl and MgProto contents in the top panel, and those of the columns ‘−CP’ in the middle columnar panel and ‘−CP−ABA’ in the bottom columnar panel are taken as 100% for stomatal apertures, respectively, for the corresponding panels. Error bars indicate s.d. (n=5).

FIG. 10 illustrates the contents of chlorophyll (Chl) and Mg-protoporphyrin IX (MgProto) are not correlated with ABA-responsive stomatal movement. Changes of Chl and MgProto (top panel) and levels of ABAR/CHLH protein (below the top panel, indicated by ‘ABAR’ and assessed by immunoblotting) after the treatment of gl1 plants with norflurazon (Nf) were compared with corresponding effects of ABA on promoting stomatal closure (middle columnar panel) and on inhibiting stomatal opening (bottom columnar panel). ABA contents (μg g⁻¹ dry weight) remained unchanged in either Nf-non-treated ([ABA]−Nf) or Nf-treated ([ABA]+Nf) plants during the period. In the top columnar panel, ‘Chla−Nf’ or ‘MgProto−Nf’ and ‘Chla+Nf’ or ‘MgProto+Nf’ denote, respectively, Chl a or MgProto contents in the Nf-non-treated (−Nf) and Nf-treated (+Nf) plants. Error bars in the ‘Chla+Nf’ columns are for total Chl (Chl a plus Chl b). In the middle columnar panel, stomatal apertures were scored before ABA treatment in the Nf-non-treated (−Nf) and Nf-treated (+Nf) plants, and they were assayed again 2 h after 10 μM (±)-ABA treatment in these −Nf(−Nf+ABA) and +Nf (+Nf+ABA) plants. In the bottom columnar panel, the stomatal initial apertures were scored in the Nf-non-treated (−Nf/init) and Nf-treated (+Nf/init) plants, and 2 h later, stomatal apertures were assayed again in the Nf-non-treated plants subjected to the ABA-free-(−Nf−ABA) or 10 μM (±)-ABA-treatment (−Nf+ABA), and also in the Nf-treated plants subjected to the ABA-free-(+Nf−ABA) or 10 μM (±)-ABA-treatment (+Nf+ABA). The contents of Chl (Chl a plus Chl b) and MgProto as well as stomatal apertures are presented in percentages relative to those of the Nf-non-treated plants, i.e., the values of the columns ‘Chla−Nf’ (Chl a plus Chl b) and ‘MgProto−Nf’ in the top panel are taken as 100%, respectively, for total Chl and MgProto contents, and those of the columns ‘−Nf’ in the middle columnar panel and ‘−Nf−ABA’ in the bottom columnar panel are taken as 100% for stomatal apertures, respectively, for the corresponding panels. Error bars indicate s.d. (n=5).

FIG. 11 illustrates down-regulation of ABAR/CHLH expression alters expression of the ABA-responsive genes in the inducible RNAi protoplast system. The protoplasts prepared from the inducible RNAi transgenic plants were incubated for 8 h in the 17beta-estradiol-containing medium (filled columns) or 17beta-estradiol-free medium (control, open columns). The expression of ABAR/CHLH and the ABA-responsive genes was assessed by real-time PCR. The values from the control protoplasts were taken as 100%, and those from the 17beta-estradiol-treated protoplasts were calculated based on the values of the control. Error bars indicate s. d. (n=5).

FIG. 12 illustrates the cch mutation decreases the activity of ABA-binding to ABAR/CHLH. [³H]ABA binding to ABAR/CHLH was assayed in the total proteins prepared from the leaves of wild-type Col plants and gun5 and cch mutants. The binding was measured by two independent assays. One is the binding assay in the 30 nM (+)-ABA-containing buffer, of which the data are expressed as nmol g⁻¹ (nmol [³H]ABA bound to 1 g total proteins), and another is the pull-down assay with anti-ABAR/CHLH serum (data expressed as d.p.m.). The amounts of ABAR/CHLH protein pulled down (indicated by arrow) from the total proteins were estimated by immunoblotting. Error bars indicate s.d. (n=3).

FIG. 13 illustrates the spatial expression of ABAR and alteration of ABA-signaling genes in transgenic plants. a, ABAR expression at the mRNA (actin mRNA as a loading control) and protein (immunodetected with anti-ABAR serum) levels in leaves (L), stems (St), siliques (Sl), flowers (Fl), roots (R), dry seeds (S1), and seeds kept at 20° C. for 24 h after stratification (S2). b, c, Real-time PCR analysis for expression of ABA-signalling genes in leaves (b) and green siliques (c) of gl1 (WT), RNAi (line 12) and overexpressing (OE line 1) plants.

### DETAILED DESCRIPTION

### I. Introduction

Based on the discovery that the Mg-chelatase H subunit (referred to herein as “CHLH” and sometimes “ABAR”)) as a receptor for abscisic acid, this invention provides a novel method for generating plants with modulated ABA sensitivity plants by increasing or decreasing the expression of CHLH in plants.

Abscisic acid is a multifunctional phytohormone involved in a variety of important protective functions including bud dormancy, seed dormancy and/or maturation, abscission of leaves and fruits, and response to a wide variety of biological stresses (e.g. cold, heat, salinity, and drought). ABA is also responsible for regulating stomatal closure by a mechanism independent of CO₂ concentration. Thus, because CHLH acts as a receptor for ABA, these phenotypes can be modulated by modulating expression of CHLH. Phenotypes that are induced by ABA can be increased or speeded in plants with increased expression of CHLH whereas such phenotypes can be reduced or slowed in plants with decreased expression of CHLH. CHLH mediates ABA signaling as a positive regulator in, for example, seed germination, post-germination growth, stomatal movement and plant tolerance to stress including, but not limited to, drought. Accordingly, when abscisic acid sensitivity is increased by overexpressing CHLH, desirable characteristics in plants such as increased stress (e.g., drought) tolerance and delayed seed germination is achieved.

### II. Mg-Chelatase H Subunit

A wide variety of CHLH polypeptide sequences are known in the art and can be used according to the methods and compositions of the invention. CHLH has been cloned and studied in a number of plant and cyanobacteria species, though never with the appreciation of the ability of the protein to function as an ABA receptor. Any known CHLH proteins, as well as active variants thereof and orthologous proteins in other plant and non-plant species can be used, as desired. As listing of some known CHLH protein and polynucleotide sequences from various species is provided in Table 1.

TABLE 1

| Plant species | Protein | Coding or cDNA sequence |
|---|---|---|
| Arabidopsis | SEQ ID NO: 2 | SEQ ID NO: 1 |
| Rice | SEQ ID NO: 4 | SEQ ID NO: 3 |
| Soybean | SEQ ID NO: 6 | SEQ ID NO: 5 |
| Barley | SEQ ID NO: 8 | SEQ ID NO: 7 |
| Snapdragon | SEQ ID NO: 10 | SEQ ID NO: 9 |
| Tobacco | SEQ ID NO: 12 | SEQ ID NO: 11 |

The present invention provides for use of the above proteins and/or nucleic acid sequences, or sequences substantially identical (e.g., 70%, 75%, 78%, 80%, 85%, 90%, 95%, 98% identical) to those listed above in the methods and compositions (e.g., expression cassettes, plants, etc.) of the present invention. In situations where variants of the above sequences are desired, it can be useful to generate sequence alignments to identify conserved amino acid or motifs (i.e., where alteration in sequences may alter protein function) and regions where variation occurs in alignment of sequences (i.e., where variation of sequence is not likely to significantly affect protein activity).

As can be seen in Table 2 below, a number of known plant CHLH polypeptides are at least about 80% identical to the Arabidopsis sequence (SEQ ID NO:2).

TABLE 2

| Organism | Aligned to | Percent Identity |
|---|---|---|
| SEQ ID NO: 2 | SEQ ID NO: 1 | 100% |
| Arabidopsis thaliana | Arabidopsis thaliana | |
| sequence Genbank | sequence Genbank | |
| #CAA92802.1 | #CAA92802.1 | |
| SEQ ID NO: 4 Oryza | SEQ ID NO: 1 | 82.3% |
| sativa sequence Genbank | Arabidopsis thaliana | |
| #ABF95686.1 | sequence Genbank | |
| | #CAA92802.1 | |
| SEQ ID NO: 6 Glycine | SEQ ID NO: 1 | 85.4% |
| max sequence Genbank | Arabidopsis thaliana | |
| #CAA04526.1 | sequence Genbank | |
| | #CAA92802.1 | |
| SEQ ID NO: 8 Hordeum | SEQ ID NO: 1 | 81.8% |
| vulgare sequence | Arabidopsis thaliana | |
| Genbank #AAK72401.1 | sequence Genbank | |
| | #CAA92802.1 | |
| SEQ ID No: 10 | SEQ ID NO: 1 | 85.2% |
| Antirrhinum majus | Arabidopsis thaliana | |
| sequence Genbank | sequence Genbank | |
| #CAA51664.1 | #CAA92802.1 | |
| SEQ ID NO: 12 Nicotiana | SEQ ID NO: 1 | 81.7% |
| tabacum sequence | Arabidopsis thaliana | |
| Genbank #AAB97152.1 | sequence Genbank | |
| | #CAA92802.1 | |

The isolation of a polynucleotide sequence encoding a plant CHLH (e.g., from plants where CHLH sequences have not yet been identified) may be accomplished by a number of techniques. For instance, oligonucleotide probes based on the CHLH coding sequences disclosed (e.g., as listed in Table 1) here can be used to identify the desired CHLH gene in a cDNA or genomic DNA library. To construct genomic libraries, large segments of genomic DNA are generated by random fragmentation, e.g., using restriction endonucleases, and are ligated with vector DNA to form concatemers that can be packaged into the appropriate vector. To prepare a cDNA library, mRNA is isolated from the desired tissue, such as a leaf from a particular plant species, and a cDNA library containing the gene transcript of interest is prepared from the mRNA. Alternatively, cDNA may be prepared from mRNA extracted from other tissues in which CHLH gene is expressed.

The cDNA or genomic library can then be screened using a probe based upon the sequence of a CHLH gene disclosed here (e.g., as listed in Table 1). Probes may be used to hybridize with genomic DNA or cDNA sequences to isolate homologous genes in the same or different plant species. Alternatively, antibodies raised against an polypeptide can be used to screen an mRNA expression library.

Alternatively, the nucleic acids encoding CHLH can be amplified from nucleic acid samples using amplification techniques. For instance, polymerase chain reaction (PCR) technology can be used to amplify the coding sequences of CHLH directly from genomic DNA, from cDNA, from genomic libraries or cDNA libraries. PCR and other in vitro amplification methods may also be useful, for example, to clone polynucleotide sequences encoding CHLH to be expressed, to make nucleic acids to use as probes for detecting the presence of the desired mRNA in samples, for nucleic acid sequencing, or for other purposes. For a general overview of PCR see PCR Protocols: A Guide to Methods and Applications. (Innis, M, Gelfand, D., Sninsky, J. and White, T., eds.), Academic Press, San Diego (1990). Appropriate primers and probes for identifying sequences from plant tissues are generated from comparisons of the sequences provided here with other related genes.

The advancement in studies of plant genomes also permits a person of skill in the art to quickly determine the CHLH coding sequence for a selected plant. For instance, the partial or entire genome of a number of plants has been sequenced and open reading frames identified. By a routine BLAST search, one can immediately identify the coding sequence for CHLH in various plants.

### III. Use of CHLH Nucleic Acids of the Invention

The invention provides methods of modulating ABA sensitivity in a plant by altering CHLH expression or activity, for example, by introducing into a plant a recombinant expression cassette comprising a regulatory element (e.g., a promoter) operably linked to a CHLH polynucleotide, i.e., a nucleic acid encoding CHLH or a sequence comprising a portion of the sequence of a CHLH mRNA or complement thereof.

In some embodiments, the methods of the invention comprise increasing and/or ectopically expressing CHLH polypeptides in a plant. Such embodiments are useful for increasing ABA sensitivity of a plant, and resulting in, for example, improved stress (e.g., drought) tolerance and/or delayed seed germination (to avoid pre-mature germination, for example as can occur in humid environments or due to other exposure to moisture). For stress tolerance, promoters can be selected that are generally constitutive and are expressed in most plant tissues, or can be leaf or root specific. To affect seed germination, promoters are generally used that result in expression in seed or, in some embodiments, floral organs or embryos.

In some embodiments, the methods of the invention comprise decreasing endogenous CHLH expression in plant, thereby decreasing ABA sensitivity in the plant. Such methods can involve, for example, mutagenesis (e.g., chemical, radiation, transposon or other mutagenesis) of CHLH sequences in a plant to reduce CHLH expression or activity, or introduction of a polynucleotide substantially identical to at least a portion of a CHLH cDNA sequence or a complement thereof (e.g., an “RNAi construct”) to reduce CHLH expression. Decreased (or increased) CHLH expression can be used to control the development of abscission zones in leaf petioles and thereby control leaf loss, i.e, delay leaf loss if expression is decreased and speed leaf loss if expression is increased in abscission zones leaf.

### A. Increasing CHLH Expression or Activity

Isolated sequences prepared as described herein can also be used to prepare expression cassettes that enhance or increase CHLH gene expression. Where overexpression of a gene is desired, the desired gene from a different species may be used to decrease potential sense suppression effects.

Any of a number of means well known in the art can be used to increase CHLH activity in plants. Any organ or plant part can be targeted, such as shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat), fruit, abscission zone, etc. Alternatively, one or several CHLH genes can be expressed constitutively (e.g., using the CaMV 35S promoter or other constitutive promoter).

One of skill will recognize that the polypeptides encoded by the genes of the invention, like other proteins, have different domains which perform different functions. Thus, the overexpressed or ectopically expressed polynucleotide sequences need not be full length, so long as the desired functional domain of the protein is expressed. Alternatively, or in addition, active CHLH proteins can be expressed as fusions, without necessarily significantly altering CHLH activity. Examples of fusion partners include, but are not limited to, poly-His or other tag sequences.

### B. Decreasing CHLH Expression or Activity

A number of methods can be used to inhibit gene expression in plants. For instance, antisense technology can be conveniently used. To accomplish this, a nucleic acid segment from the desired gene is cloned and operably linked to a promoter such that the antisense strand of RNA will be transcribed. The expression cassette is then transformed into plants and the antisense strand of RNA is produced. In plant cells, it has been suggested that antisense RNA inhibits gene expression by preventing the accumulation of mRNA which encodes the enzyme of interest, see, e.g., Sheehy et al., Proc. Nat. Acad. Sci. USA, 85:8805-8809 (1988); Pnueli et al., The Plant Cell 6:175-186 (1994); and Hiatt et al., U.S. Pat. No. 4,801,340.

The antisense nucleic acid sequence transformed into plants will be substantially identical to at least a portion of the endogenous gene or genes to be repressed. The sequence, however, does not have to be perfectly identical to inhibit expression. Thus, an antisense or sense nucleic acid molecule encoding only a portion of CHLH, or a portion of the CHLH cDNA, can be useful for producing a plant in which CHLH expression is suppressed. The vectors of the present invention can be designed such that the inhibitory effect applies to other proteins within a family of genes exhibiting homology or substantial homology to the target gene.

For antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. For example, a sequence of between about 30 or 40 nucleotides can be used, and in some embodiments, about full length nucleotides should be used, though a sequence of at least about 20, 50 100, 200, or 500 nucleotides can be used.

Catalytic RNA molecules or ribozymes can also be used to inhibit expression of CHLH genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs.

A number of classes of ribozymes have been identified. One class of ribozymes is derived from a number of small circular RNAs that are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes is described in Haseloff et al. Nature, 334:585-591 (1988).

Another method of suppression is sense suppression (also known as co-suppression). Introduction of expression cassettes in which a nucleic acid is configured in the sense orientation with respect to the promoter has been shown to be an effective means by which to block the transcription of target genes. For an example of the use of this method to modulate expression of endogenous genes see, Napoli et al., The Plant Cell 2:279-289 (1990); Flavell, Proc. Natl. Acad. Sci., USA 91:3490-3496 (1994); Kooter and Mol, Current Opin. Biol. 4:166-171 (1993); and U.S. Pat. Nos. 5,034,323, 5,231,020, and 5,283,184.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity can exert a more effective repression of expression of the endogenous sequences. In some embodiments, sequences with substantially greater identity are used, e.g., at least about 80, at least about 95%, or 100% identity are used. As with antisense regulation, the effect can be designed and tested to apply to any other proteins within a similar family of genes exhibiting homology or substantial homology.

For sense suppression, the introduced sequence in the expression cassette, needing less than absolute identity, also need not be full length, relative to either the primary transcription product or fully processed mRNA. This may be preferred to avoid concurrent production of some plants that are overexpressers. A higher identity in a shorter than full length sequence compensates for a longer, less identical sequence. Furthermore, the introduced sequence need not have the same intron or exon pattern, and identity of non-coding segments will be equally effective. Normally, a sequence of the size ranges noted above for antisense regulation is used, i.e., 30-40, or at least about 20, 50, 100, 200, 500 or more nucleotides.

Endogenous gene expression may also be suppressed by means of RNA interference (RNAi) (and indeed co-suppression can be considered a type of RNAi), which uses a double-stranded RNA having a sequence identical or similar to the sequence of the target gene. RNAi is the phenomenon in which when a double-stranded RNA having a sequence identical or similar to that of the target gene is introduced into a cell, the expressions of both the inserted exogenous gene and target endogenous gene are suppressed. The double-stranded RNA may be formed from two separate complementary RNAs or may be a single RNA with internally complementary sequences that form a double-stranded RNA. Although complete details of the mechanism of RNAi are still unknown, it is considered that the introduced double-stranded RNA is initially cleaved into small fragments, which then serve as indexes of the target gene in some manner, thereby degrading the target gene. RNAi is known to be also effective in plants (see, e.g., Chuang, C. F. & Meyerowitz, E. M., Proc. Natl. Acad. Sci. USA 97: 4985 (2000); Waterhouse et al., Proc. Natl. Acad. Sci. USA 95:13959-13964 (1998); Tabara et al. Science 282:430-431 (1998); Matthew, Comp Funct Genom 5: 240-244 (2004); Lu, et al., Nucleic Acids Research 32(21):e171 (2004)). For example, to achieve suppression of the expression of a DNA encoding a protein using RNAi, a double-stranded RNA having the sequence of a DNA encoding the protein, or a substantially similar sequence thereof (including those engineered not to translate the protein) or fragment thereof, is introduced into a plant of interest. The resulting plants may then be screened for a phenotype associated with the target protein and/or by monitoring steady-state RNA levels for transcripts encoding the protein. Although the genes used for RNAi need not be completely identical to the target gene, they may be at least 70%, 80%, 90%, 95% or more identical to the target gene sequence. See, e.g., U.S. Patent Publication No. 2004/0029283. The constructs encoding an RNA molecule with a stem-loop structure that is unrelated to the target gene and that is positioned distally to a sequence specific for the gene of interest may also be used to inhibit target gene expression. See, e.g., U.S. Patent Publication No. 2003/0221211.

The RNAi polynucleotides can encompass the full-length target RNA or may correspond to a fragment of the target RNA. In some cases, the fragment will have fewer than 100, 200, 300, 400, 500 600, 700, 800, 900 or 1,000 nucleotides corresponding to the target sequence. In addition, in some embodiments, these fragments are at least, e.g., 50, 100, 150, 200, or more nucleotides in length. In some cases, fragments for use in RNAi will be at least substantially similar to regions of a target protein that do not occur in other proteins in the organism or may be selected to have as little similarity to other organism transcripts as possible, e.g., selected by comparison to sequences in analyzing publicly-available sequence databases.

Expression vectors that continually express siRNA in transiently- and stably-transfected have been engineered to express small hairpin RNAs, which get processed in vivo into siRNAs molecules capable of carrying out gene-specific silencing (Brummelkamp et al., Science 296:550-553 (2002), and Paddison, et al., Genes & Dev. 16:948-958 (2002)). Post-transcriptional gene silencing by double-stranded RNA is discussed in further detail by Hammond et al. Nature Rev Gen 2: 110-119 (2001), Fire et al. Nature 391: 806-811 (1998) and Timmons and Fire Nature 395: 854 (1998).

One of skill in the art will recognize that using technology based on specific nucleotide sequences (e.g., antisense or sense suppression technology), families of homologous genes can be suppressed with a single sense or antisense transcript. For instance, if a sense or antisense transcript is designed to have a sequence that is conserved among a family of genes, then multiple members of a gene family can be suppressed. Conversely, if the goal is to only suppress one member of a homologous gene family, then the sense or antisense transcript should be targeted to sequences with the most variance between family members.

Another means of inhibiting CHLH function in a plant is by creation of dominant negative mutations. In this approach, non-functional, mutant CHLH polypeptides, which retain the ability to interact with wild-type subunits are introduced into a plant. A dominant negative construct also can be used to suppress CHLH expression in a plant. A dominant negative construct useful in the invention generally contains a portion of the complete CHLH coding sequence sufficient, for example, for DNA-binding or for a protein-protein interaction such as a homodimeric or heterodimeric protein-protein interaction but lacking the transcriptional activity of the wild type protein.

### IV. Recombinant Expression Vector

Once the coding or cDNA sequence for CHLH is obtained, it can also be used to prepare an expression cassette for expressing the CHLH protein in a transgenic plant, directed by a heterologous promoter. Increased expression of CHLH polynucleotide is useful, for example, to produce plants with enhanced drought-resistance. Alternatively, as described above, expression vectors can also be used to express CHLH polynucleotides and variants thereof that inhibit endogenous CHLH expression.

Any of a number of means well known in the art can be used to increase or decrease CHLH activity or expression in plants. Any organ can be targeted, such as shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit. Alternatively, the CHLH gene can be expressed constitutively (e.g., using the CaMV 35S promoter).

To use CHLH coding or cDNA sequences in the above techniques, recombinant DNA vectors suitable for transformation of plant cells are prepared. Techniques for transforming a wide variety of higher plant species are well known and described in the technical and scientific literature. See, e.g., Weising et al. Ann. Rev. Genet. 22:421-477 (1988). A DNA sequence coding for the CHLH polypeptide preferably will be combined with transcriptional and translational initiation regulatory sequences which will direct the transcription of the sequence from the gene in the intended tissues of the transformed plant.

For example, a plant promoter fragment may be employed to direct expression of the CHLH gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill.

Alternatively, the plant promoter may direct expression of the CHLH protein in a specific tissue (tissue-specific promoters) or may be otherwise under more precise environmental control (inducible promoters). Examples of tissue-specific promoters under developmental control include promoters that initiate transcription only in certain tissues, such as leaves or guard cells (including but not limited to those described in WO/2005/085449; U.S. Pat. No. 6,653,535; Li et al., Sci China C Life Sci. 2005 April; 48(2):181-6; Husebye, et al., Plant Physiol, April 2002, Vol. 128, pp. 1180-1188; and Plesch, et al., Gene, Volume 249, Number 1, 16 May 2000, pp. 83-89(7)). Examples of environmental conditions that may affect transcription by inducible promoters include anaerobic conditions, elevated temperature, or the presence of light.

If proper protein expression is desired, a polyadenylation region at the 3′-end of the coding region should be included. The polyadenylation region can be derived from the natural gene, from a variety of other plant genes, or from T-DNA.

The vector comprising the sequences (e.g., promoters or CHLH coding regions) will typically comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or Basta.

In some embodiments, the CHLH nucleic acid sequence is expressed recombinantly in plant cells to enhance and increase levels of total CHLH polypeptide. A variety of different expression constructs, such as expression cassettes and vectors suitable for transformation of plant cells can be prepared. Techniques for transforming a wide variety of higher plant species are well known and described in the technical and scientific literature. See, e.g., Weising et al. Ann. Rev. Genet. 22:421-477 (1988). A DNA sequence coding for a CHLH protein can be combined with cis-acting (promoter) and trans-acting (enhancer) transcriptional regulatory sequences to direct the timing, tissue type and levels of transcription in the intended tissues of the transformed plant. Translational control elements can also be used.

The invention provides a CHLH nucleic acid operably linked to a promoter which, in some embodiments, is capable of driving the transcription of the CHLH coding sequence in plants. The promoter can be, e.g., derived from plant or viral sources. The promoter can be, e.g., constitutively active, inducible, or tissue specific. In construction of recombinant expression cassettes, vectors, transgenics, of the invention, a different promoters can be chosen and employed to differentially direct gene expression, e.g., in some or all tissues of a plant or animal.

A. Constitutive Promoters

A promoter fragment can be employed to direct expression of a CHLH nucleic acid in all transformed cells or tissues, e.g., as those of a regenerated plant. The term “constitutive regulatory element” means a regulatory element that confers a level of expression upon an operatively linked nucleic molecule that is relatively independent of the cell or tissue type in which the constitutive regulatory element is expressed. A constitutive regulatory element that is expressed in a plant generally is widely expressed in a large number of cell and tissue types. Promoters that drive expression continuously under physiological conditions are referred to as “constitutive” promoters and are active under most environmental conditions and states of development or cell differentiation.

A variety of constitutive regulatory elements useful for ectopic expression in a transgenic plant are well known in the art. The cauliflower mosaic virus 35S (CaMV 35S) promoter, for example, is a well-characterized constitutive regulatory element that produces a high level of expression in all plant tissues (Odell et al., Nature 313:810-812 (1985)). The CaMV 35S promoter can be particularly useful due to its activity in numerous diverse plant species (Benfey and Chua, Science 250:959-966 (1990); Futterer et al., Physiol. Plant 79:154 (1990); Odell et al., supra, 1985). A tandem 35S promoter, in which the intrinsic promoter element has been duplicated, confers higher expression levels in comparison to the unmodified 35S promoter (Kay et al., Science 236:1299 (1987)). Other useful constitutive regulatory elements include, for example, the cauliflower mosaic virus 19S promoter; the Figwort mosaic virus promoter; and the nopaline synthase (nos) gene promoter (Singer et al., Plant Mol. Biol. 14:433 (1990); An, Plant Physiol. 81:86 (1986)).

Additional constitutive regulatory elements including those for efficient expression in monocots also are known in the art, for example, the pEmu promoter and promoters based on the rice Actin-1 5′ region (Last et al., Theor. Appl. Genet. 81:581 (1991); Mcelroy et al., Mol. Gen. Genet. 231:150 (1991); Mcelroy et al., Plant Cell 2:163 (1990)). Chimeric regulatory elements, which combine elements from different genes, also can be useful for ectopically expressing a nucleic acid molecule encoding a CHLH protein (Comai et al., Plant Mol. Biol. 15:373 (1990)).

Other examples of constitutive promoters include the 1′- or 2′-promoter derived from T-DNA of Agrobacterium tumafaciens (see, e.g., Mengiste (1997) supra; O'Grady (1995) Plant Mol. Biol. 29:99-108); actin promoters, such as the Arabidopsis actin gene promoter (see, e.g., Huang (1997) Plant Mol. Biol. 1997 33:125-139); alcohol dehydrogenase (Adh) gene promoters (see, e.g., Millar (1996) Plant Mol. Biol. 31:897-904); ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol. 208:551-565 (1989)), Gpc2 from maize (GenBank No. U45855, Manjunath et al., Plant Mol. Biol. 33:97-112 (1997)), other transcription initiation regions from various plant genes known to those of skill. See also Holtorf Plant Mol. Biol. 29:637-646 (1995).

B. Inducible Promoters

Alternatively, a plant promoter may direct expression of the CHLH gene under the influence of changing environmental conditions or developmental conditions. Examples of environmental conditions that may effect transcription by inducible promoters include anaerobic conditions, elevated temperature, drought, or the presence of light. Such promoters are referred to herein as “inducible” promoters. For example, the invention can incorporate drought-specific promoter such as the drought-inducible promoter of maize (Busk (1997) supra); or alternatively the cold, drought, and high salt inducible promoter from potato (Kirch (1997) Plant Mol. Biol. 33:897-909).

Alternatively, plant promoters which are inducible upon exposure to plant hormones, such as auxins, are used to express the CHLH gene. For example, the invention can use the auxin-response elements E1 promoter fragment (AuxREs) in the soybean (Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant. Microbe Interact. 10:933-937); and, the promoter responsive to the stress hormone abscisic acid (Sheen (1996) Science 274:1900-1902).

Plant promoters inducible upon exposure to chemicals reagents that may be applied to the plant, such as herbicides or antibiotics, are also useful for expressing the CHLH gene. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. A CHLH coding sequence can also be under the control of, e.g., a tetracycline-inducible promoter, e.g., as described with transgenic tobacco plants containing the Avena sativa L. (oat) arginine decarboxylase gene (Masgrau (1997) Plant J 11:465-473); or, a salicylic acid-responsive element (Stange (1997) Plant J. 11:1315-1324; Uknes et al., Plant Cell 5:159-169 (1993); Bi et al., Plant J. 8:235-245 (1995)).

Examples of useful inducible regulatory elements include copper-inducible regulatory elements (Mett et al., Proc. Natl. Acad. Sci. USA 90:4567-4571 (1993); Furst et al., Cell 55:705-717 (1988)); tetracycline and chlor-tetracycline-inducible regulatory elements (Gatz et al., Plant J. 2:397-404 (1992); Röder et al., Mol. Gen. Genet. 243:32-38 (1994); Gatz, Meth. Cell Biol. 50:411-424 (1995)); ecdysone inducible regulatory elements (Christopherson et al., Proc. Natl. Acad. Sci. USA 89:6314-6318 (1992); Kreutzweiser et al., Ecotoxicol. Environ. Safety 28:14-24 (1994)); heat shock inducible regulatory elements (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example, IPTG-inducible expression (Wilde et al., EMBO J. 11:1251-1259 (1992)). An inducible regulatory element useful in the transgenic plants of the invention also can be, for example, a nitrate-inducible promoter derived from the spinach nitrite reductase gene (Back et al., Plant Mol. Biol. 17:9 (1991)) or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)).

C. Tissue-Specific Promoters

Alternatively, the plant promoter may direct expression of the CHLH gene in a specific tissue (tissue-specific promoters). Tissue specific promoters are transcriptional control elements that are only active in particular cells or tissues at specific times during plant development, such as in vegetative tissues or reproductive tissues.

Examples of tissue-specific promoters under developmental control include promoters that initiate transcription only (or primarily only) in certain tissues, such as vegetative tissues, e.g., roots or leaves, or reproductive tissues, such as fruit, ovules, seeds, pollen, pistols, flowers, or any embryonic tissue. Reproductive tissue-specific promoters may be, e.g., ovule-specific, embryo-specific, endosperm-specific, integument-specific, seed and seed coat-specific, pollen-specific, petal-specific, sepal-specific, or some combination thereof.

Other tissue-specific promoters include seed promoters. Suitable seed-specific promoters are derived from the following genes: MAC1 from maize (Sheridan (1996) Genetics 142:1009-1020); Cat3 from maize (GenBank No. L05934, Abler (1993) Plant Mol. Biol. 22:10131-1038); vivparous-1 from Arabidopsis (Genbank No. U93215); atmycl from Arabidopsis (Urao (1996) Plant Mol. Biol. 32:571-57; Conceicao (1994) Plant 5:493-505); napA from Brassica napus (GenBank No. J02798, Josefsson (1987) JBL 26:12196-1301); and the napin gene family from Brassica napus (Sjodahl (1995) Planta 197:264-271).

A variety of promoters specifically active in vegetative tissues, such as leaves, stems, roots and tubers, can also be used to express the CHLH gene. For example, promoters controlling patatin, the major storage protein of the potato tuber, can be used, see, e.g., Kim (1994) Plant Mol. Biol. 26:603-615; Martin (1997) Plant J. 11:53-62. The ORF13 promoter from Agrobacterium rhizogenes that exhibits high activity in roots can also be used (Hansen (1997) Mol. Gen. Genet. 254:337-343. Other useful vegetative tissue-specific promoters include: the tarin promoter of the gene encoding a globulin from a major taro (Colocasia esculenta L. Schott) corm protein family, tarin (Bezerra (1995) Plant Mol. Biol. 28:137-144); the curculin promoter active during taro corm development (de Castro (1992) Plant Cell 4:1549-1559) and the promoter for the tobacco root-specific gene TobRB7, whose expression is localized to root meristem and immature central cylinder regions (Yamamoto (1991) Plant Cell 3:371-382).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier (1997) FEBS Lett. 415:91-95). A ribulose bisphosphate carboxylase promoters expressed almost exclusively in mesophyll cells in leaf blades and leaf sheaths at high levels, described by Matsuoka (1994) Plant J. 6:311-319, can be used. Another leaf-specific promoter is the light harvesting chlorophyll a/b binding protein gene promoter, see, e.g., Shiina (1997) Plant Physiol. 115:477-483; Casal (1998) Plant Physiol. 116:1533-1538. The Arabidopsis thaliana myb-related gene promoter (Atmyb5) described by Li (1996) FEBS Lett. 379:117-121, is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize by Busk (1997) Plant J. 11:1285-1295, can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, described by Di Laurenzio (1996) Cell 86:423-433; and, Long (1996) Nature 379:66-69; can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto (1995) Plant Cell. 7:517-527). Also useful are kn1-related genes from maize and other species which show meristem-specific expression, see, e.g., Granger (1996) Plant Mol. Biol. 31:373-378; Kerstetter (1994) Plant Cell 6:1877-1887; Hake (1995) Philos. Trans. R. Soc. Lond. B. Biol. Sci. 350:45-51. For example, the Arabidopsis thaliana KNAT1 promoter (see, e.g., Lincoln (1994) Plant Cell 6:1859-1876).

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In another embodiment, the CHLH gene is expressed through a transposable element. This allows for constitutive, yet periodic and infrequent expression of the constitutively active polypeptide. The invention also provides for use of tissue-specific promoters derived from viruses including, e.g., the tobamovirus subgenomic promoter (Kumagai (1995) Proc. Natl. Acad. Sci. USA 92:1679-1683; the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer (1996) Plant Mol. Biol. 31:1129-1139).

### IV. Production of Transgenic Plants

As detailed herein, the present invention provides for transgenic plants comprising recombinant expression cassettes either for expressing CHLH proteins in a plant or for inhibiting or reducing endogenous CHLH expression. Thus, in some embodiments, a transgenic plant is generated that contains a complete or partial sequence of an endogenous CHLH encoding polynucleotide, either for increasing or reducing CHLH expression and activity. In some embodiments, a transgenic plant is generated that contains a complete or partial sequence of a polynucleotide that is substantially identical to an endogenous CHLH encoding polynucleotide, either for increasing or reducing CHLH expression and activity. In some embodiments, a transgenic plant is generated that contains a complete or partial sequence of a polynucleotide that is from a species other than the species of the transgenic plant. It should be recognized that transgenic plants encompass the plant or plant cell in which the expression cassette is introduced as well as progeny of such plants or plant cells that contain the expression cassette, including the progeny that have the expression cassette stably integrated in a chromosome.

A recombinant expression vector comprising a CHLH coding sequence driven by a heterologous promoter may be introduced into the genome of the desired plant host by a variety of conventional techniques. For example, the DNA construct may be introduced directly into the genomic DNA of the plant cell using techniques such as electroporation and microinjection of plant cell protoplasts, or the DNA construct can be introduced directly to plant tissue using ballistic methods, such as DNA particle bombardment. Alternatively, the DNA construct may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. While transient expression of CHLH is encompassed by the invention, generally expression of construction of the invention will be from insertion of expression cassettes into the plant genome, e.g., such that at least some plant offspring also contain the integrated expression cassette.

Microinjection techniques are also useful for this purpose. These techniques are well known in the art and thoroughly described in the literature. The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

Agrobacterium tumefaciens-mediated transformation techniques, including disarming and use of binary vectors, are well described in the scientific literature. See, for example, Horsch et al. Science 233:496-498 (1984), and Fraley et al. Proc. Natl. Acad. Sci. USA 80:4803 (1983).

Transformed plant cells derived by any of the above transformation techniques can be cultured to regenerate a whole plant that possesses the transformed genotype and thus the desired phenotype such as enhanced drought-resistance. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

The expression cassettes of the invention can be used to confer drought resistance on essentially any plant. Thus, the invention has use over a broad range of plants, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and, Zea. In some embodiments, the plant is selected from the group consisting of rice, maize, wheat, soybeans, cotton, canola, and alfalfa. In some embodiments, the plant is an ornamental plant. In some embodiment, the plant is a vegetable- or fruit-producing plant.

In some embodiments, the methods of the invention are used to confer drought-resistance on turf grasses. A number of turf grasses are known to those of skill in the art. For example, fescue, Festuca spp. (e.g., F. arundinacea, F. rubra, F. ovina var. duriuscula, and F. ovina) can be used. Other grasses include Kentucky bluegrass Poa pratensis and creeping bentgrass Agrostis palustris.

Those of skill will recognize that a number of plant species can be used as models to predict the phenotypic effects of transgene expression in other plants. For example, it is well recognized that both tobacco (Nicotiana) and Arabidopsis plants are useful models of transgene expression, particularly in other dicots.

The plants of the invention have either enhanced or reduced abscisic acid sensitivity compared to plants are otherwise identical except for expression of CHLH. Abscisic acid sensitivity can be monitored by observing or measuring any phenotype mediated by ABA. Those of skill in the art will recognize that ABA is a well-studied plant hormone and that ABA mediates many changes in characteristics, any of which can be monitored to determined whether ABA sensitivity has been modulated. In some embodiments, modulated ABA sensitivity is manifested by altered timing of seed germination or altered stress (e.g., drought) tolerance.

Drought resistance can assayed according to any of a number of well-known techniques. For example, plants can be grown under conditions in which less than optimum water is provided to the plant. Drought resistance can be determined by any of a number of standard measures including turgor pressure, growth, yield, and the like. In some embodiments, the methods described in the Example section, below can be conveniently used.

### EXAMPLES

The following examples are offered to illustrate, but not to limit the claimed invention.

The phytohormone ABA has a vital function in plant adaptation to stressful environments by regulating stomatal aperture and the expression of stress-responsive genes, and in plant development such as seed maturation, germination and seedling growth (Leung, J. et al., Annu. Rev. Plant Physiol. Plant Mol. Biol. 49:199-222 (1998); Finkelstein, R. R. et al., Plant Cell S15-S45 (2002); Himmelbach, A. et al., Curr. Opin. Plant Biol. 6:470-479 (2003)). Genetic approaches have permitted the characterization of numerous components involved in ABA signalling but have failed to identify ABA receptors (Leung, J. et al., Annu. Rev. Plant Physiol. Plant Mol. Biol. 49:199-222 (1998); Finkelstein, R. R. et al., Plant Cell S15-S45 (2002); Himmelbach, A. et al., Curr. Opin. Plant Biol. 6:470-479 (2003)). Biochemical approaches provide another way to isolate ABA receptors by the identification of ABA-binding proteins that are putative ABA receptors (Homberg, C. et al., Nature 310:321-324 (1984); Zhang, D. P. et al., Plant physiol. 128:714-725 (2002); Razem, F. A. et al., J. Biol. Chem. 279:9922-99′29 (2004)). Recently, the RNA-binding protein FCA, a homologue of an ABA-binding protein ABAP1 (Razem, F. A. et al., J. Biol. Chem. 279:9922-9929 (2004)), was identified as an ABA receptor in the regulation of flowering time (Razem, F. A. et al., Nature 439:290-294 (2006)). However, ABA receptors involved in seed development, seedling growth and stomatal movement remain elusive.

We previously reported an ABA-specific-binding protein from broad bean (Vicia faba) and found that this protein was potentially involved in ABA-induced stomatal signaling (Zhang, D. P. et al., Plant physiol. 128:714-725 (2002)). So we designated it ABAR (for putative abscisic acid receptor). On the basis of the sequencing information, we isolated from broad bean leaves a complementary DNA fragment encoding the carboxy-terminal half of about 770 amino acids of the putative H subunit (CHLH) of the magnesium-protoporphyrin-IX chelatase (Mg-chelatase). CHLH has been reported to have multiple functions in plant cells. In addition to its enzymatic functions as a subunit of the Mg-chelatase in producing photosynthetic apparatus (Walker, C. J. et al., Biochem. J. 327:321-333 (1997)), CHLH has a key function in mediating plastid-to-nucleus signaling (Mochizuki, N. et al., Proc. Natl. Acad. Sci. USA 98:2053-2058 (2001); Surpin, M., et al., Plant Cell S327-S338 (2002); Strand, A., et al., Nature 421:79-83 (2003); Nott, A. et al., Annu. Rev. Plant Biol. 57:730-759 (2006)). We found that the yeast-expressed product of the cDNA fragment encoding the C-terminal portion of the broad bean ABAR or CHLH binds ABA specifically (data not shown). The information from Vicia faba led us to analyse the functions of ABAR in ABA signalling in Arabidopsis thaliana. Here we show that the Arabidopsis ABAR/CHLH (the Arabidopsis genomic locus tag for CHLH is At5 g13630, and the GenBank accession numbers are AY070133, BT002311, NM_{—}121366, Z68495 or AY078971) is an ABA receptor that regulates seed development, post-germination growth and stomatal aperture.

### Experimental Procedures

### Plant Materials, Constructs for and Generation of Transgenic Plants, and Growth Conditions.

Arabidopsis thaliana ecotype gl1 was used in the generation of transgenic plants. The gl1 is Arabidopsis thaliana ecotype Columbia carrying the homozygous recessive glabrous mutation. We used gl1 because the plants of gl1 grow better in our experimental conditions. To create transgenic plant lines overexpressing ABAR/CHLH gene (Arabidopsis genomic locus tag: At5g13630), the open reading frame (ORF) for the ABAR/CHLH gene was isolated by polymerase chain reaction (PCR) using the forward primer 5′-TAGGCGCGCCAAAATGGCTTCGCTTGTGTATTCTCC-3′ (SEQ ID NO:13) and reverse primer 5′-GGACTAGTTTATCGATCGATCCCTTCGATCTTGTC-3′ (SEQ ID NO:14). The cauliflower mosaic virus (CaMV) 35S:ABAR chimeric gene construct was generated by ligating the ORF (4146 bp) of ABAR/CHLH gene into the pGSA1276 vector (http://www.arabidopsis.org/abrc/catalog/vector_{—}1) by AscI and SpeI sites. To create ABAR/CHLH antisense lines, a gene-specific DNA fragment covering parts of 5′-untranslated region and coding region (corresponding to nucleotides-125 to 948) of the full length ABAR cDNA was amplified with the forward primer 5′-ACGGGTACCGAGAGAATCATAAACTCCCACTTGG-3′ (SEQ ID NO:15) and reverse primer 5′-TCGTCTAGAGAGTGAGTCATTGGTGTCCCTTC-3′ (SEQ ID NO:16). The fragment of 1073 bp was inserted inversely into the vector of a pCAMBIA-1300-based Super promoter (Leung, J. et al., Annu. Rev. Plant Physiol. Plant Mol. Biol. 49:199-222 (1998)) by KpnI and XbalI sites under the control of the Super promoter. To generate RNA interference (RNAi) lines down-regulating ABAR/CHLH expression, a gene-specific 653-bp fragment amplified with forward primer 5′-CGGTCTAGAACAGAGATTCTGTGGTTGGG-3′ (SEQ ID NO:17) and reverse primer 5′-ATACCCGGGGGCACTTGCCATTGCTGCTGTT-3′ (SEQ ID NO:18) which locates downstream 2363 to 3015 bp of the start codon and has Xbal I and SmaI restriction sites was used as sense arm, and a 596-bp fragment amplified with forward primer 5′-ATAGAGCTCTTTTGCCGTGCGGGCTTCACGT-3′ (SEQ ID NO:19) and reverse primer 5′-CTGTACGTAGGCACTTGCCATTGCTGCTGTT-3′ (SEQ ID NO:20) which locates downstream 2420 to 3015 bp of the start codon and has SacI and SnaBI sites was used as anti-sense arm. The PCR fragments were ligated, respectively, into pBI121 vector (13.0 kb, Clontech) under the control of CaMV 35S promoter by corresponding restriction sites. To generate the chemical-regulated inducible RNAi lines under-expressing ABAR/CHLH upon induction by beta-estradiol (Finkelstein, R. R. et al., Plant Cell S15-S45 (2002)), a gene-specific 351-bp fragment corresponding to the region of nt 843 to 1193 of the ABAR/CHLH cDNA was PCR-amplified with forward primer 5′-CCGCTCGAGGTTCTTGGATACTGGAATTTGG-3′ (SEQ ID NO:21) and reverse primer 5′-ACGCGTCGACGGTCCACCAACAAGAGCAAAAC-3′ (SEQ ID NO:22). This fragment was inserted in sense orientation into the XhoI/SalI sites of pSK-int vector as described previously (Finkelstein, R. R. et al., Plant Cell S15-S45 (2002)). The same fragment, amplified with forward primer 5′-CGGAATTCGGTCCACCAACAAGAGCAAAAC-3′ (SEQ ID NO:23) and reverse primer 5′-GACTAGTGTTCTTGGATACTGGAATTTGG-3′ (SEQ ID NO:24), was subsequently placed in antisense orientation into the EcoRI/SpeI sites of pSK-int already carrying the sense fragment. Finally, the entire RNAi cassette comprising the sense and antisense fragments interspersed by the actin II intron was excised from pSK-int using the flanking SpeI/XhoI sites and inserted into the SpeI/XhoI site of pX7-GFP vector yielding the construct pX7-ABARi.

The sequences of the constructs for the stable expression and inducible RNAi and antisense-ABAR/CHLH described above are all listed in the Table 3.

TABLE 3

The sequences of the constructs of the stable
expression and inducible RNAi and anitsense-
ABAR/CHLH

1. The sequence of the stable RNAi construct
Sense arm (SEQ ID NO:25): nt 2363-3015 in the
Arabidopsis ABAR/CHLH full length cDNA; total
length: 653 bp. Letters in italics indicate the
primer sequences.
ACAGAGATTCTGTGGTTGGGAAAGTTTATTCCAAGATTATGGAGATTGAA
TCAAGGCTTTTGCCGTGCGGGCTTCACGTCATTGGAGAGCCTCCATCCGC
CATGGAAGCTGTGGCCACACTGGTCAACATTGCTGCTCTAGATCGTCCGG
AGGATGAGATTTCAGCTCTTCCTTCTATATTAGCTGAGTGTGTTGGAAGG
GAGATAGAGGATGTTTACAGAGGAAGCGACAAGGGTATCTTGAGCGATGT
AGAGCTTCTCAAAGAGATCACTGATGCCTCACGTGGCGCTGTTTCCGCCT
TTGTGGAAAAAACAACAAATAGCAAAGGACAGGTGGTGGATGTGTCTGAC
AAGCTTACCTCGCTTCTTGGGTTTGGAATCAATGAGCCATGGGTTGAGTA
TTTGTCCAACACCAAGTTCTACAGGGCGAACAGAGATAAGCTCAGAACAG
TGTTTGGTTTCCTTGGAGAGTGCCTGAAGTTGGTGGTCATGGACAACGAA
CTAGGGAGTCTAATGCAAGCTTTGGAAGGCAAGTACGTCGAGCCTGGCCC
CGGAGGTGATCCCATCAGAAACCCAAAGGTCTTACCAACCGGTAAAAACA
TCCATGCCTTAGATCCTCAGGCTATTCCCACAACAGCAGCAATGGCAAGT
GCC
Antisense arm (SEQ ID NO:26): nt 2420-3015 in the
Arabidopsis ABAR/CHLH full length cDNA; total
length: 596 bp. Letters in italics indicate the
primer sequences.
TTTTGCCGTGCGGGCTTCACGTCATTGGAGAGCCTCCATCCGCCATGGAA
GCTGTGGCCACACTGGTCAACATTGCTGCTCTAGATCGTCCGGAGGATGA
GATTTCAGCTCTTCCTTCTATATTAGCTGAGTGTGTTGGAAGGGAGATAG
AGGATGTTTACAGAGGAAGCGACAAGGGTATCTTGAGCGATGTAGAGCTT
CTCAAAGAGATCACTGATGCCTCACGTGGCGCTGTTTCCGCCTTTGTGGA
AAAAACAACAAATAGCAAAGGACAGGTGGTGGATGTGTCTGACAAGCTTA
CCTCGCTTCTTGGGTTTGGAATCAATGAGCCATGGGTTGAGTATTTGTCC
AACACCAAGTTCTACAGGGCGAACAGAGATAAGCTCAGAACAGTGTTTGG
TTTCCTTGGAGAGTGCCTGAAGTTGGTGGTCATGGACAACGAACTAGGGA
GTCTAATGCAAGCTTTGGAAGGCAAGTACGTCGAGCCTGGCCCCGGAGGT
GATCCCATCAGAAACCCAAAGGTCTTACCAACCGGTAAAAACATCCATGC
CTTAGATCCTCAGGCTATTCCCACAACAGCAGCAATGGCAAGTGCC
Loop sequence (a fragment of GUS gene) (SEQ ID NO:
27): Letters in italics indicate SmaI and SnaBI
sites, respectively.
CCCGGGTGGTCAGTCCCTTATGTTACGTCCTGTAGAAACCCCAACCCGTG
AAATCAAAAAACTCGACGGCCTGTGGGCATTCAGTCTGGATCGCGAAAAC
TGTGGAATTGATCAGCGTTGGTGGGAAAGCGCGTTACAAGAAAGCCGGGC
AATTGCTGTGCCAGGCAGTTTTAACGATCAGTTCGCCGATGCAGATATTC
GTAATTATGCGGGCAACGTCTGGTATCAGCGCGAAGTCTTTATACCGAAA
GGTTGGGCAGGCCAGCGTATCGTGCTGCGTTTCGATGCGGTCACTCATTA
CGGCAAAGTGTGGGTCAATAATCAGGAAGTGATGGAGCATCAGGGCGGCT
ATACGCCATTTGAAGCCGATGTCACGCCGTATGTTATTGCCGGGAAAAGT
GTACGTA.
2. The sequence of the chemical-regulated
inducible RNAi construct (SEQ ID NO:28)
The nt 843-1193 in the Arabidopsis ABAR/CHLH full
length cDNA; total length: 351 bp. The sequence
for the sense arm is the same as that for anti-
sense arm. Letters in italics indicate the
sequences of the primers.
GTTCTTGGATACTGGAATTTGGCATCCACTTGCTCCAACCATGTACGATG
ATGTGAAGGAGTACTGGAACTGGTATGACACTAGAAGGGACACCAATGAC
TCACTCAAGAGGAAAGATGCAACGGTTGTCGGTTTAGTCTTGCAGAGGAG
TCACATTGTGACTGGTGATGATAGTCACTATGTGGCTGTTATCATGGAGC
TTGAGGCTAGAGGTGCTAAGGTCGTTCCTATATTCGCAGGAGGGTTGGAT
TTCTCTGGTCCAGTAGAGAAATATTTCGTAGACCCGGTGTCGAAACAGCC
CATCGTAAACTCTGCTGTCTCCTTGACTGGTTTTGCTCTTGTTGGTGGAC
C
Loop sequence: A fragment (about 300 bp) of Arabi-
dopsis Actin II intron sequence.
3. The sequence of the antisense-ABAR/CHLH
construct (SEQ ID NO:29)
The nt-125 to 948 upstream of and within the open
reading frame of the Arabidopsis ABAR/CHLH full
length cDNA; total length: 1073 bp. Letters in
italics indicate the sequences of the primers.
GAGAGAATCATAAACTCCCACTTGGAGCTCAAAAAGTGTAAGAGACAACC
AACAAAAAACGATTCATCTCTTCTCCTATCCTCTCCTCTTCGAATTCAAC
GTTTGGAGAATCCAGCAGCCGCAAAATGGCTTCGCTTGTGTATTCTCCAT
TCACTCTATCCACTTCTAAAGCAGAGCATCTCTCTTCGCTCACTAACAGT
ACCAAACATTCTTTCCTCCGGAAGAAACACAGATCAACCAAACCAGCCAA
ATCTTTCTTCAAGGTGAAATCTGCTGTATCTGGAAACGGCCTCTTCACAC
AGACGAACCCGGAGGTCCGTCGTATAGTTCCGATCAAGAGAGACAACGTT
CCGACGGTGAAAATCGTCTACGTCGTCCTCGAGGCTCAGTACCAGTCTTC
TCTCAGTGAAGCCGTGCAATCTCTCAACAAGACTTCGAGATTCGCATCCT
ACGAAGTGGTTGGATACTTGGTCGAGGAGCTTAGAGACAAGAACACTTAC
AACAACTTCTGCGAAGACCTTAAAGACGCCAACATCTTCATTGGTTCTCT
GATCTTCGTCGAGGAATTGGCGATTAAAGTTAAGGATGCGGTGGAGAAGG
AGAGAGACAGGATGGACGCAGTTCTTGTCTTCCCTTCAATGCCTGAGGTA
ATGAGACTGAACAAGCTTGGATCTTTTAGTATGTCTCAATTGGGTCAGTC
AAAGTCTCCGTTTTTCCAACTCTTCAAGAGGAAGAAACAAGGCTCTGCTG
GTTTTGCCGATAGTATGTTGAAGCTTGTTAGGACTTTGCCTAAGGTTTTG
AAGTACTTACCTAGTGACAAGGCTCAAGATGCTCGTCTCTACATCTTGAG
TTTACAGTTTTGGCTTGGAGGCTCTCCTGATAATCTTCAGAATTTTGTTA
AGATGATTTCTGGATCTTATGTTCCGGCTTTGAAAGGTGTCAAAATCGAG
TATTCGGATCCGGTTTTGTTCTTGGATACTGGAATTTGGCATCCACTTGC
gTCCAACCATGTACGATGATGTGAAGGAGTACTGGAACTGGTATGACACTA
GAAGGGACACCAATGACTCACTC

These constructs were introduced in Agrobacterium tumefaciens GV3101 strain and transformed into plants by floral infiltration. The homozygous T3 seeds of the transgenic plants were used for analysis. For the inducible RNAi, ten putative transgenic lines were tested by Northern and Western blot after application of 10 μM 17beta-estradiol (Sigma), and all the ten lines showed significant decline of ABAR transcript and product. The seeds of the gun4-1, gun5-1 and cch mutants were a generous gift from Dr. J. Chory (The Salk Institute, La Jolla, Calif.). The seeds of the mutants hy1-1 (ABRC#: CS67), hy2-1 (CS68) and chl-2 (CS3362) were obtained from the Arabidopsis Biological Resource Center (ABRC). Except for the mutants hy1-1 and hy2-1 with the ecotype Ler as background, all the other mutants were isolated from the ecotype Columbia. Plants were grown in a growth chamber at 20-21° C. on Murashige-Skoog (MS) medium at about 80 μmol photons m⁻² s⁻¹, or in compost soil at about 120 μmol photons m⁻² s⁻¹ over a 16-h photoperiod.

### Expression of Recombinant ABAR/CHLH in Yeast and Purification of the Fusion Protein.

Full-length ABAR/CHLH coding sequence was PCR-amplified with forward primer 5′-TCGTCGACAAAATGGCTTCGCTTGTGTATTCTCC-3′ (SEQ ID NO:30) and reverse primer 5′-TAGCGGCCGCTATCGATCGATCCCTTCGATCTTGTC-3′ (SEQ ID NO:31). The recombinant ABAR/CHLH was expressed in yeast as a fusion protein with a 6×His (SEQ ID NO:32) tag by using Pichia Methanolica Expression kit (Invitrogen) and the fusion protein was affinity-purified according to manufacturer's instructions.

### ABA Binding Assays.

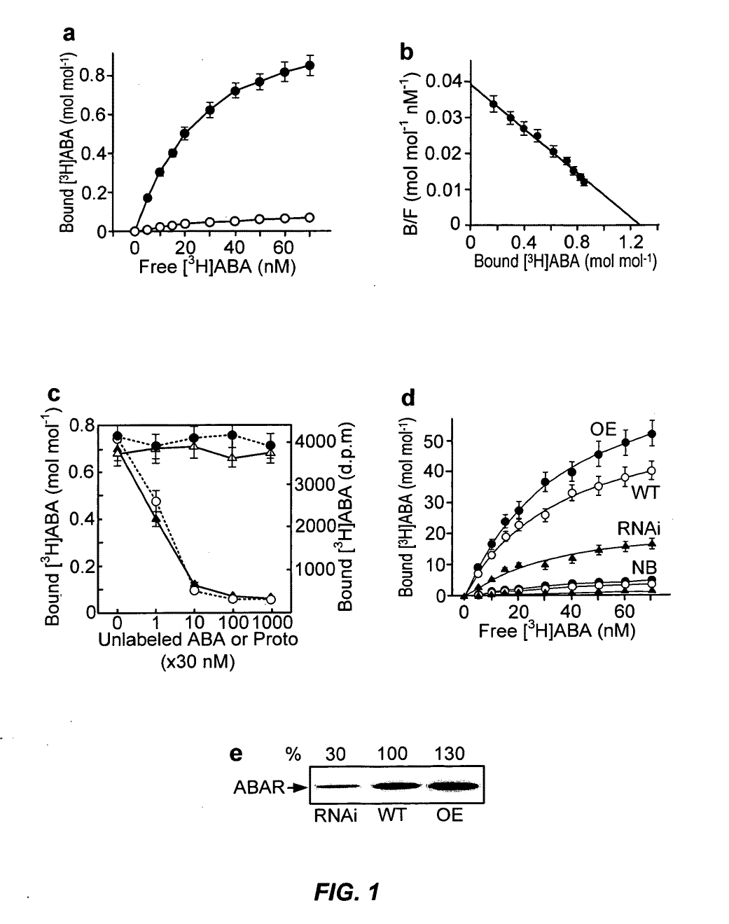

The ABA-binding activity of ABAR was assayed according to the previously described procedures (Zhang, D. P. et al., Plant physiol. 128:714-725 (2002)) with modifications. The binding medium (medium A) contained 50 mM Tris-HCl (pH 7.0), 2 mM MgCl₂, 1 mM CaCl₂, and 250 mM mannitol. The medium for extracting the Arabidopsis natural ABAR/CHLH protein (medium B) was the medium A supplemented with 2 mM 1,4-dithiothreitol (DTT), 1 mM phenylmethyl sulfonyl fluoride (PMSF), 10 μg/μl leupeptin, and 10 μl/μl pepstin A but minus 250 mM mannitol. [³H](+)ABA (American Radiolabeled Chemicals, 2.37×10¹² Bq mmol⁻¹, purity 98.4%) at 30 nM, or at a step gradient of concentrations from 0 to 70 nM (when analyzing ABA binding kinetics, see FIG. 1a, d1), together with 2 μg purified yeast-expressed ABAR/CHLH protein or 50 μg proteins of the crude extracts from two-week-old Arabidopsis seedlings, was added into the medium A. The total volume for the binding assays was adjusted to 200 μl. The mixtures were incubated at 25° C. for 30 min, and then quickly placed on ice. Following the addition of 100 μl 0.5% (w/v) Dextran T70-coated charcoal (DCC) to remove the free [³H]ABA by adsorption, the mixtures were maintained on ice for 5 min, and then centrifuged to remove DCC before the radioactivity in the supernatant was counted. The specific binding was determined by the difference between the radioactivity bound to the immuno-purified ABAR protein or crude extracts of Arabidopsis seedlings incubated only with [³H]ABA (total binding) and radioactivity bound in the presence of 1,000-fold molar excess of unlabeled (+)-ABA (Sigma; non-specific binding. Non-specific binding in all the assays was lower than 10% of the total binding, see FIG. 1a, d1). The unlabeled ABA was added into the incubation medium at the same time with [³H]ABA. The ABA-binding activity of the crude ABA-binding protein from Arabidopsis seedling was expressed as the number of nanomoles of [³H]ABA specifically bound per gram of protein, and that of the immuno-purified ABAR protein as the number of moles of [³H]ABA per mole of protein. The stereo-specificity of the ABA-binding was assayed as described previously (Zhang, D. P. et al., Plant physiol. 128:714-725 (2002)). The two inactive ABA isomers, (−)-ABA and trans-ABA (Sigma), together with (+)-ABA (as controls), were used to compete possibly for the same binding sites of the proteins. The conditions of incubation were the same as described above (the incubation medium containing 30 nM [³H]ABA), and the two ABA isomers and (+)-ABA were assayed in the concentrations ranging from 1 to 1000-fold molar excess of [³H]ABA.

We conducted a preliminary experiment of ABA binding by using the transgenic yeast extracts, which showed that the extracts from the yeasts expressing ABAR cDNA specifically bound ABA (Kd=36 nM), but the controls (the cell-free protein extracts from either the yeasts transformed by the same empty vector or non-transgenic yeast lines) did not. In addition, we observed that neither the denatured ABAR protein by boiling nor bovine serum albumin (BSA) showed any binding activity to ABA (data not shown), indicating that ABA binding requires active ABAR.

The pull down assay was done as follows to detect ABA binding to the natural ABAR/CHLH protein. Total proteins were extracted from two-week-old Arabidopsis seedlings with the medium C composed of the medium B supplemented with 250 mM mannitol. The crude extracts (3 mg total proteins) was incubated by gently shaking in the medium C (400 μl) containing 30 nM [³H](+)ABA at 4° C. for 2 h, and the anti-ABAR^{N} serum (10 μg, described below) was added into the medium for a further incubation at 4° C. for 2 h, and then the medium was supplemented by 100 μl 25% (v/v) protein A-agarose (Santa Cruz) for a final incubation at 4° C. for 2 h. Following the incubation, protein A-agarose was recovered by a brief centrifugation and washed with 1 ml medium C. The pellets were resuspended in 100 ml water and mixed with scintillation fluid before the radioactivity (d.p.m., disintegrations per minute) of the bound [³H](+)ABA was measured.

Three independent control experiments were performed for the pull down assays. A control was done by addition of the same amounts of mouse preimmune serum instead of the antiserum to the medium of the above-described pull down assay in the extracts from the leaves of wild-type plants. In the second control experiment, [³H]ABA binding was assayed in the binding medium A as described above with the supernatants obtained after the precipitation of the wild-type plant extracts with the preimmune serum (at 4° C. for 2 h). Depleting ABAR protein with the anti-ABAR serum from the wild-type plant extracts was taken as the third control. The anti-ABAR serum (20 μg) was added to the extracts for incubation at 4° C. for 2 h to deplete ABAR protein from the total proteins, and the supernatants deprived of ABAR protein were obtained after removing protein A-agarose-antiserum-ABAR protein complexes by centrifugation. These supernatants were used to assay either [³H]ABA binding or residual ABAR protein by immunoblotting with anti-ABAR serum.

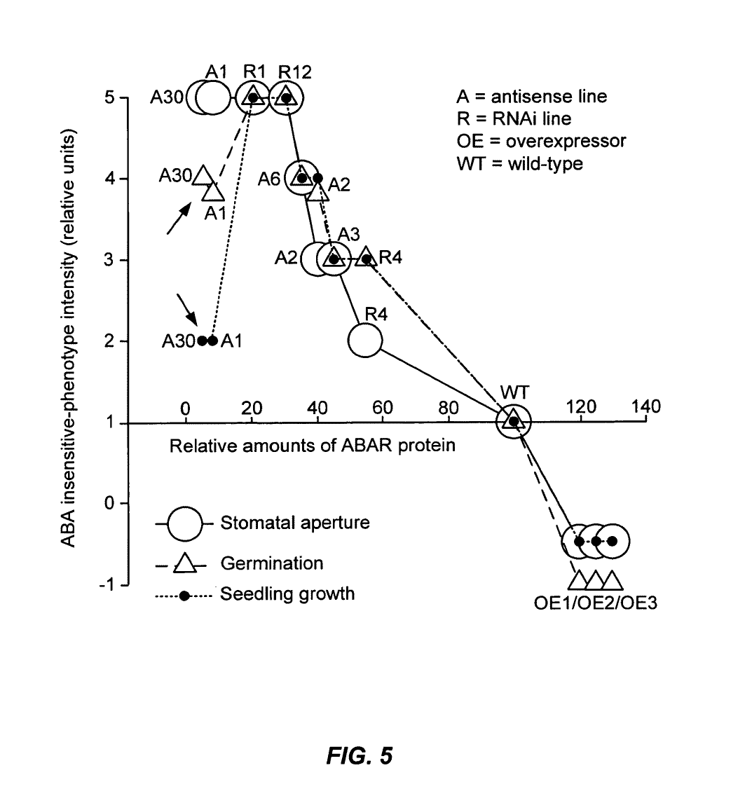

For the assays of ABA binding in transgenic ABAR-RNAi and -overexpressor plants, we used more than three lines for each type of the transgenic plants and the results obtained were similar. Thus, only were the data with RNAi line 12 and overexpressor line 1 (see FIG. 5) shown in the text.

### T-DNA Insertion Knockout Mutation in the ABAR/CHLH Gene.

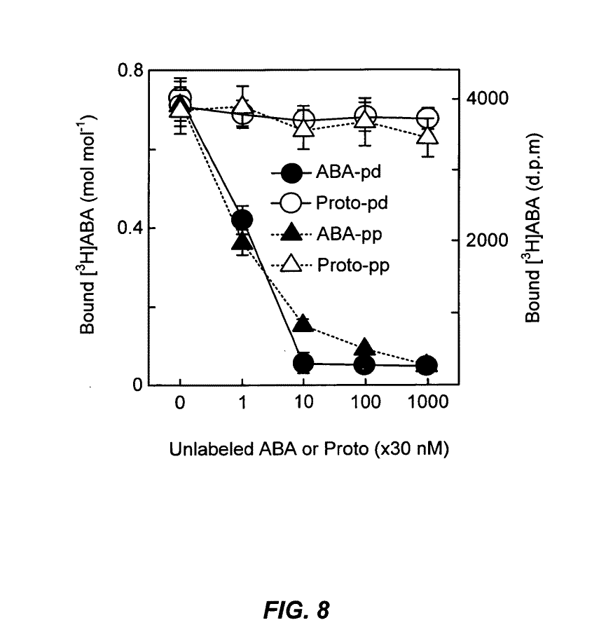

T-DNA insertion lines in the ABAR/CHLH gene in Colombia ecotype were obtained from the Salk Institute (http://signal.salk.edu/) through ABRC. The screening for the knockout mutants was done following the recommended procedures. We identified a T-DNA insertion allele (SALK_{—}062726) in the 1st exon of the ABAR/CHLH gene (FIG. 8), designated abar-1.

The heterozygous ABAR/abar-1 plants grew as well as their background wild-type, but produced both the normally-germinating seeds and late- or non-germinating seeds at a ratio of 3:1. Of all the abar-1 seeds, less than 10% germinated, and the albino seedlings died after one week in MS-medium. The phenotypes of the abar-1 seeds were rescued by introducing into the heterozygous ABAR/abar-1 plants the wild-type ABAR/CHLH gene included in a 7.6-kb Sal I-Sph I fragment from MSH12 that contains the genomic sequence of ABAR/CHLH (data not shown), showing that the abar-1 phenotypes in seeds are caused by defects in the ABAR/CHLH gene.

### Phenotypic Analysis.

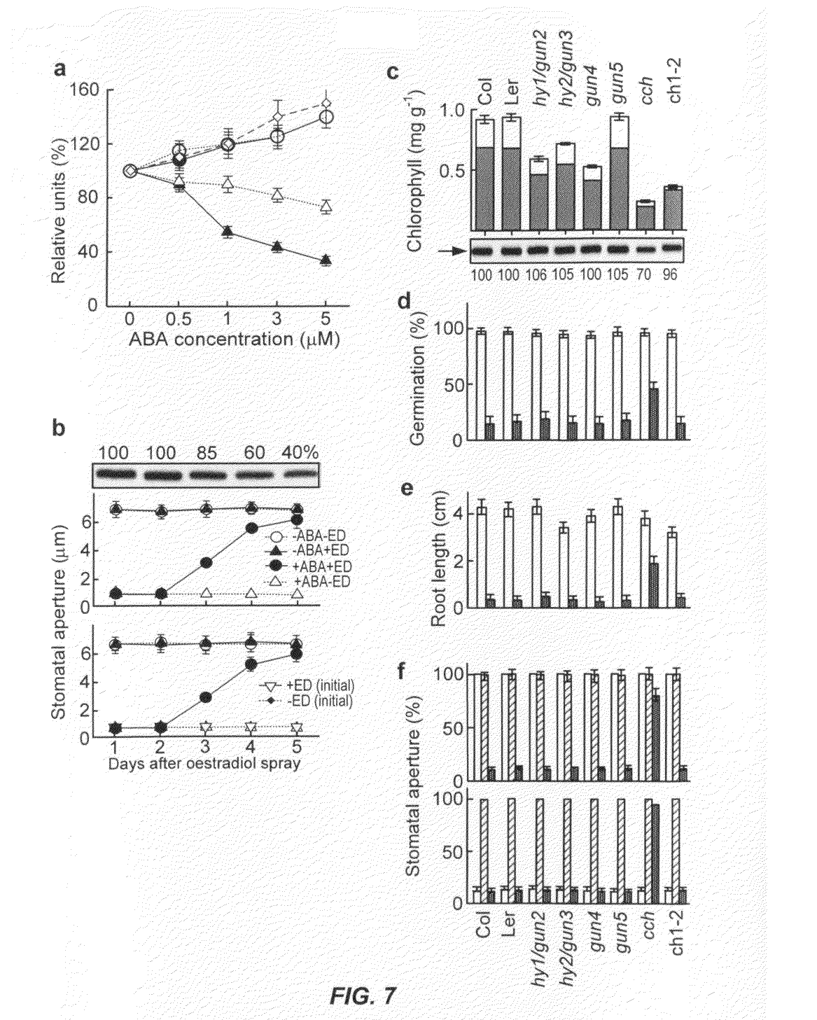

For germination assay, approximately 100 seeds each from wild types (gl1, Colombia or Ler) and mutants or transgenic mutants were planted in triplicate on MS medium (Sigma, product#, M5524) with or without different concentrations of (±)-ABA and incubated at 4° C. for 3 days before being placed 20° C. under light conditions, and germination (emergence of radicals) was scored at the indicated times. For seedling growth experiment, seeds were germinated after stratification on common MS medium and 48 h later transferred to MS medium supplemented with different concentrations of ABA in the vertical position. Seedling growth was observed 10 days after the transfer. It should be noted that the phenotypes in ABA-responsive post-germination growth in the mutants used in this experiment were observed only if the seedlings were transferred to the ABA-containing medium less than 48 h after stratification, but it was not observed when the transfer was done more than 48 h after stratification (data not shown). This phenomenon may be due to a post-germination developmental arrest checkpoint mediated by temporal expression of ABI5 (Lopez-Molina, L. et al., Proc. Natl. Acad. Sci. USA 98:4782-4787 (2001)). For drought tolerance experiment, plants were grown aseptically in Petri dishes containing selective agar germination medium for 2 weeks, and then transferred to 8-cm compost-soil-filled pots. 15 d later when plantlets reached the stage of five to six fully expanded leaves, drought was imposed by withdrawing irrigation for one-half of the plants until the lethal effects was observed on most of these plants, whereas the other half were grown under a standard irrigation regime as a control. For water loss assay, rosette leaves were detached from their roots, placed on filter paper, and left on the lab bench. The loss in fresh weight was monitored at the indicated times. For stomatal aperture assays, leaves were floated in the buffer containing 50 mM KCl and 10 mM Mes-Tris (pH 6.15) under a halogen cold-light source (Colo-Parmer) at 200 μmol m⁻² sec⁻¹ for 2 hr followed by addition of different concentrations of (±)-ABA. Apertures were recorded on epidermal strips after 2 h of further incubation to estimate ABA-induced closure. To study inhibition of opening, leaves were floated on the same buffer in the dark for 2 hr before they were transferred to the cold-light for 2 h in the presence of ABA, and then apertures were determined. For the assays of stomatal response to ABA with different mutants, it is necessary to note the maximum stomatal apertures (100%, see FIG. 7f) in the experimental conditions (μm): 4.7 for hy1/gun2; 4.6 for hy2/gun3; 4.6 for gun4; 4.2 for gun5; 5.1 for cch; 5.1 for ch1-2; 5.2 for the wild-type Col; 4.8 for the wild type Ler.

For the phenotype analysis in transgenic ABAR-RNAi and -overexpressor plants, we used more than ten lines for each type of the transgenic plants and the results obtained were similar. Thus, only were the data with RNAi line 12 and overexpressor line 1 (see FIG. 5) shown in the text.

### RNA Gel Blotting, Reverse Transcriptase-Mediated PCR and Real-Time PCR.

RNA gel blotting for ABAR/CHLH expression was done essentially as described previously (Yu, X. C. et al., Plant Physiol 140:558-579 (2006)) by using forward primer 5′-CTGAGTGTGTTGGAAGGGAGATAGA-3′ (SEQ ID NO:33) and reverse primer 5′-CTCTACCAACCTCTCAACCACAATC-3′ (SEQ ID NO:34) for PCR-amplification of the gene-specific probes. The mRNA band intensity was estimated by densitometric scans of the bands using a digital imaging system. To analyze the expression of ABAR/CHLH by reverse transcriptase-mediated PCR, the gene-specific primers for ABAR/CHLH were 5′-CCGCTCGAGGTTCTTGGATACTGGAATTTGG-3′ (SEQ ID NO:21) (forward) and 5′-ACGCGTCGACGGTCCACCAACAAGAGCAAAAC-3′ (SEQ ID NO:22) (reverse). Real-time PCR for mRNA expression of various ABA-signaling genes (see Table 1 for the gene-specific primers) was performed according to the instructions provided for the DNA Engine Opticon 2 Thermal Cycler (MJ Research) with SYBR Premix Ex Taq system (Takara).

TABLE 4
Gene-specific primers for real time PCR analysis.

| | Arabidopsis | | SEQ |
|---|---|---|---|
| Gene | genomic | | ID |
| name | locus tag | Forward primer and reverse primer | NO: |
| RD29A | At5g52310 | 5′-ATCACTTGGCTCCACTGTTGTTC-3′ and | 35 |
| | | 5′-ACAAAACACACATAAACATCCAAAGT-3′ | 36 |
| MYB2 | At2g47190 | 5′-TGCTCGTTGGAACCACATCG-3′ and | 37 |
| | | 5′-ACCACCTATTGCCCCAAAGAGA-3′ | 38 |
| MYC2 | At1g32640 | 5′-TCATACGACGGTTGCCAGAA-3′ and | 39 |
| | | 5′-AGCAACGTTTACAAGCTTTGATTG-3′ | 40 |
| OST1 | At4g33950 | 5′-TGGAGTTGCGAGATTGATGAGAG-3′ and | 41 |
| | | 5′-CCTGTGGTTGATTATCTCCCTTTTT-3′ | 42 |
| ABI1 | At4g26080 | 5′-AGAGTGTGCCTTTGTATGGTTTTA-3′ and | 43 |
| | | 5′-CATCCTCTCTCTACAATAGTTCGCT-3′ | 44 |
| ABI2 | At5g57050 | 5′-GATGGAAGATTCTGTCTCAACGATT-3′ and | 45 |
| | | 5′-GTTTCTCCTTCACTATCTCCTCCG-3′ | 46 |
| ABI3 | At3g24650 | 5′-TCCATTAGACAGCAGTCAAGGTTT-3′ and | 47 |
| | | 5′-GGTGTCAAAGAACTCGTTGCTATC-3′ | 48 |
| ABI4 | At2g40220 | 5′-GGGCAGGAACAAGGAGGAAGTG-3′ and | 49 |
| | | 5′-ACGGCGGTGGATGAGTTATTGAT-3′ | 50 |
| ABI5 | At2g36270 | 5′-CAATAAGAGAGGGATAGCGAACGAG-3′ and | 51 |
| | | 5′-CGTCCATTGCTGTCTCCTCCA-3′ | 52 |
| CIPK15 | At5g01810 | 5′-CAGAGAAGGAAAAGAAGCGGTG-3′ and | 53 |
| | | 5′-CTCCTCCTTCTCCTCTCCCTTCT-3′ | 54 |
| EM1 | At3g51810 | 5′-CAAAGCAACTGAGCAGAGAAGAGC-3′ and | 55 |
| | | 5′-CCTCCCTTGCTCCTTCCTTCA-3′ | 56 |
| EM6 | At2g40170 | 5′-CAGCAGATGGGACGCAAAGG-3′ and | 57 |
| | | 5′-TATTACATCCGTGTGGGGAAGTTTG-3′ | 58 |

For the expression analysis of the ABA signaling genes in transgenic ABAR-RNAi and -overexpressor plants, we used more than three lines for each type of the transgenic plants and the results obtained were similar. Thus, only were the data with RNAi line 12 and overexpressor line 1 (see FIG. 5) shown in the text.

### Production of Anti-ABAR/CHLH Serum.

A fragment of ABAR/CHLH cDNA corresponding to the N-terminal 258 amino acids (from 52 to 310) was isolated using forward primer 5′-TTAGAATTCGGAAACGGCCTCTTCACACAGAC-3′ (SEQ ID NO:59) and reverse primer 5′-CGCGTCGACTCCCTTCTAGTGTCATACCAGTTCCAG-3′ (SEQ ID NO:60) and expressed in E. coli as glutathione S-transferase-ABAR^{N} fusion protein. The affinity-purified fusion protein was used for standard immunization protocols in mouse. The antiserum was affinity-purified and shown to be highly specific.

### Immunoblotting and Immunolabeling.

The immunoblotting of the total proteins with anti-ABAR^{N} serum was done essentially according to the previously described procedures (Yu, X. C. et al., Plant Physiol 140:558-579 (2006)). Protein band intensity was estimated by densitometric scans of the bands using a digital imaging system. For immunolabeling of ABAR protein in seeds, the sections prepared from the paraffin-embedded seeds were incubated with anti-ABAR^{N} serum and goat anti-mouse IgG conjugated with fluorescein isothiocyanate (MP Biomedicals), and observed under a confocal laser scanning microscope.

### Chlorophyll and Porphyrin Measurements.

The contents of chlorophyll, protoporphyrin IX and Mg-protoporphyrin IX were assayed essentially by the previously described procedures (Mochizuki, N. et al., Proc. Natl. Acad. Sci. USA 98:2053-2058 (2001)).

### Assays of the Effects of ABA on ABAR/CHLH Expression and Mg-Chelatase Activity.

For the effects of ABA in vivo, the seeds of the ecotype gl1 were germinated after stratification on common MS medium and 48 h later transferred to MS medium supplemented with different concentrations of ABA. 10 d later, the contents of chlorophyll and porphyrins including of protoporphyrin IX and Mg-protoporphyrin IX were measured. At the same time total proteins were extracted from leaves for testing the amounts of ABAR/CHLH protein by immunoblotting.

For the effects of ABA on Mg-chelatase activity in vitro, isolation of intact chloroplasts from 3-week-old gl1 plants and assays of both plasmid intactness and Mg-chelatase activity were done essentially according to the method of Walker and Weinstein (Walker, C. J. et al., Plant Physiol. 95:1189-1196 (1991); Walker, C. J. et al., Proc. Natl. Acad. Sci. USA 88:5789-5793 (1991)). Briefly, the purified chloroplasts with high degree of intactness were pre-incubated for 20 min in the chloroplast isolation buffer supplemented with different concentrations of (±)-ABA. For assaying Mg-chelatase activity, reactions were started by addition of the ABA-pretreated plastids and terminated by addition of ice-cold acetone. All the manipulations were performed under dim light to prevent porphyrin-mediated photooxidative damage. After centrifugation, the green supernatant was reserved and diluted with 4 ml of 75% (v/v) acetone. The fluorescence of the product was read directly in this acetone extract.

### Induction of RNAi.

For the treatment of seedlings of the inducible RNAi lines with 17beta-estradiol (Sigma) to induce down-regulation of ABAR/CHLH expression, 17beta-estradiol was diluted from 10 mM stock solution prepared in dimethyl sulfoxide (DMSO) to 10 μM, and equivalent volume of DMSO was included in the 17beta-estradiol-free-treated controls. Three-week old T3 RNAi seedlings were treated with 10 μM 17beta-estradiol by spraying intact plants (single spray for each set of experiments). The contents of chlorophyll, protoporphyrin IX and Mg-protoporphyrin IX and stomatal response to exogenous (±)-ABA were assayed at different time intervals after the 17beta-estradiol application. The amounts of ABAR/CHLH protein were also determined at the same time by immunoblotting. For the treatment of protoplasts of the inducible RNAi lines, the manipulations were conducted under dim light. The protoplasts were prepared from leaves of the inducible RNAi plants as described by Sheen at http://genetics.mgh.harvard.edu/sheenweb. The protoplasts of high quality were incubated for 8 h in the medium containing 400 mM mannitol, 15 mM MgCl₂, 4 mM Mes (pH5.7) and 2 μM 17beta-estradiol, and equivalent volume of DMSO was included in the 17beta-estradiol-free-treated controls. The expression of ABAR/CHLH and some ABA-responsive genes was assessed by real-time PCR.

Treatments with Norflurazon and Chloramphenicol.

The reagent chloramphenicol (Sigma; CP) was diluted from 100 mg/ml stock solutions prepared in ethanol to 150 μg/ml, and equivalent volume of ethanol was included in the chloramphenicol-free-treated controls. For norflurazon (Sigma; Nf) treatment, Nf was diluted from 100 mM stock solutions prepared in DMSO to 3 μM, and equivalent volume of DMSO was included in the Nf-free-treated controls. The 3-week-old seedlings were treated with CP or Nf by irrigation of either 150 μg/ml CP or 3 μM Nf dissolved both in distilled water. Stomatal response to exogenous (±)-ABA was assayed at different time intervals after the application of the reagents, and the amounts of ABAR/CHLH protein were immuno-determined at the same time.

### ABA Analysis.

ABA contents in tissues were assayed by radioimmunoassay method as described previously (Zhang, D. P. et al., Plant physiol. 128:714-725 (2002)).

### Results

### ABAR Specifically Binds ABA

The purified yeast-expressed Arabidopsis ABAR binds ABA as a saturable process (FIG. 1a). The ABAR protein possesses one binding site, as shown by a linear Scatchard plot and a maximum binding (Bmax) of 1.28 mol ABA per mol of protein, and has a high binding affinity for ABA, as shown by its equilibrium dissociation constant (Kd) of 32 nM (FIG. 1b). Furthermore, the purified ABAR binds ABA in a highly stereospecific manner, which was revealed by the efficient displacement of [3H](+)-ABA binding by the physiologically active form (+)-ABA but not by two inactive ABA isomers, (−)-ABA and trans-ABA, which are structurally similar to (+)-ABA (FIG. 1c; data for trans-ABA not shown).

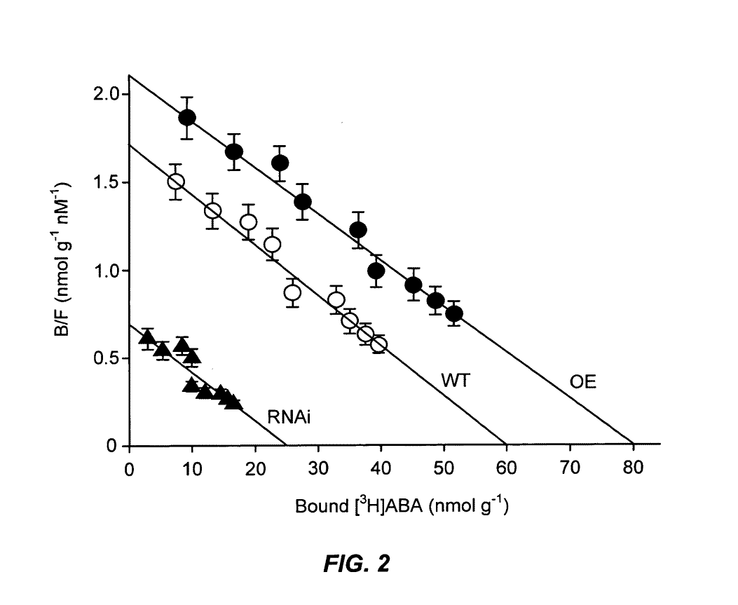

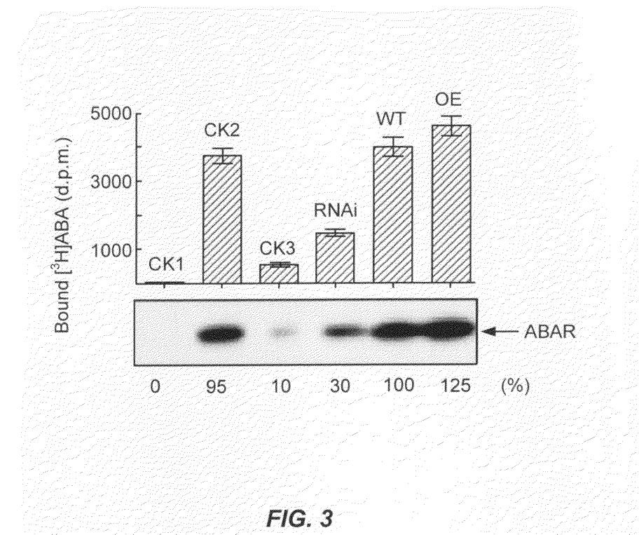

We further analysed the ABA-binding ability of natural ABAR protein. The saturable process of ABA binding to the extracts of leaves was found (FIG. 1d). Upregulation of the ABAR level by overexpressing ABAR enhanced, but downregulation by RNA-mediated interference (RNAi) reduced, the Bmax of ABA binding, whereas neither substantially changed Kd (from 35 to 38 nM) (FIG. 1d, e, and FIG. 2), revealing that the changes in ABAR abundance alter the numbers of ABA-binding sites but do not modify the affinity. A pull-down assay with the ABAR-specific antiserum specifically co-precipitated the ABA-binding activities proportionally to the amounts of the ABAR protein (FIG. 3), and the pulled-down ABA-binding activity was shown to be highly stereospecific for (+)-ABA (FIG. 1c; data for trans-ABA not shown). The data reveal that ABAR specifically binds ABA, and the binding meets primary criteria for an ABA receptor.

### ABAR Mediates ABA Signaling as a Positive Regulator

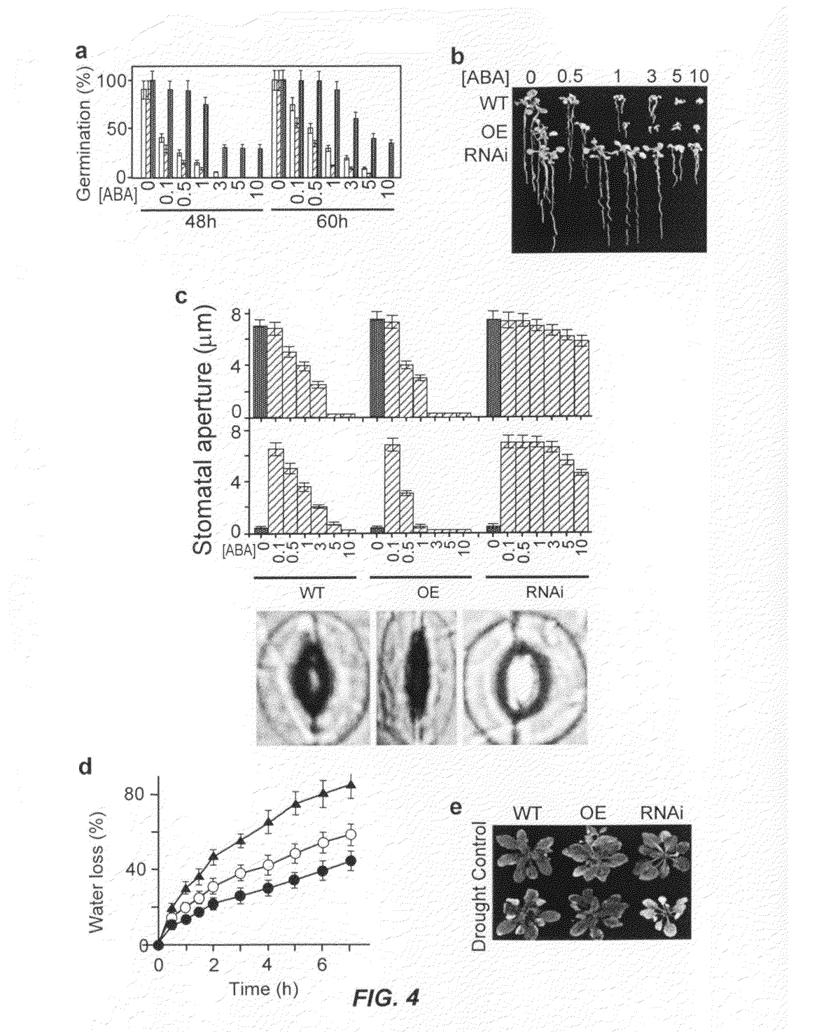

To explore the functions of ABAR in ABA signalling, we generated Arabidopsis transgenic RNAi, antisense and overexpression lines. The plants underexpressing ABAR as a result of RNAi showed significant ABA-insensitive phenotypes in seed germination (FIG. 4a), post-germination growth arrest by ABA (FIG. 4b) and ABA-induced promotion of stomatal closure and inhibition of stomatal opening (FIG. 4c). In contrast, the plants overexpressing ABAR displayed the ABA-hypersensitive phenotypes (FIG. 4a-c) and were more resistant to dehydration from their leaves or whole plants, but the RNAi plants were more sensitive to dehydration (FIG. 4d, e). Overall, the ABAR levels were negatively correlated with the intensity of the ABA-insensitive phenotypes (FIG. 5). The ABA concentrations did not change in the transgenic plants (in the range of 0.2 mug g-l dry weight), showing that ABAR is not involved in ABA biosynthesis.

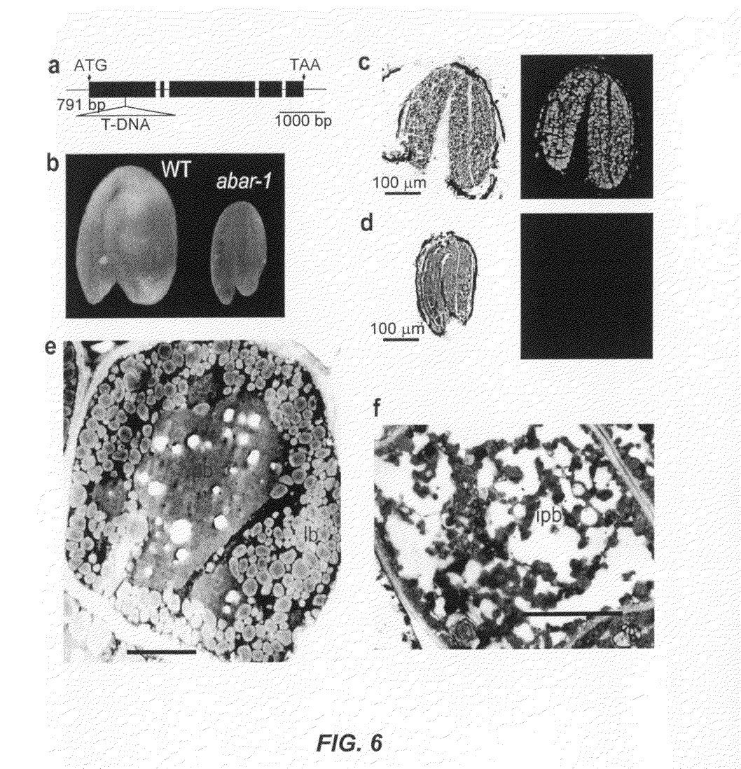

We further identified a transferred DNA (T-DNA) insertion mutant in the ABAR gene (FIG. 6), designated abar-1. Homozygous abar-1 is lethal. The abar-1 seeds are deficient in lipid and mature protein bodies (FIG. 6), indicating a possible distortion of late embryonic development (Finkelstein, R. R. et al., Plant Cell S15-S45 (2002)). These phenotypes are similar to those of the mutations in ABA-signalling genes such as ABI3, which has specific effects on seed maturation (Giraudat, J. et al., Plant Cell 4:1251-1261 (1992); Nambara, E. et al., Plant J 2:435-441 (1992)). Taken together, the data show that ABAR mediates ABA signalling as a positive regulator.

### ABAR-Mediated ABA Signaling is a Distinct Process

Mg-chelatase, which is composed of three subunits, namely CHLD, CHLI and CHLH, catalyses the insertion of Mg2+ into protoporphyrin-IX (Proto) to form Mg-protoporphyrin-IX (MgProto), the first step unique to chlorophyll synthesis (Walker, C. J. et al., Biochem. J. 327:321-333 (1997)). CHLH has a central function as a monomeric Proto-binding protein (Walker, C. J. et al., Biochem. J. 327:321-333 (1997); Karger, G. A. et al., Biochemistry 40:9291-9299 (2001)). The Arabidopsis genomes uncoupled 5 (gun5) mutant, resulting in a single amino acid Ala 990right arrowVal mutation in CHLH, showed that CHLH is involved in plastid-to-nucleus retrograde signalling by controlling the metabolism of the tetrapyrrole signal MgProto or by sensing the signal (Mochizuki, N. et al., Proc. Natl. Acad. Sci. USA 98:2053-2058 (2001); Surpin, M., et al., Plant Cell S327-S338 (2002); Strand, A., et al., Nature 421:79-83 (2003); Nott, A. et al., Annu. Rev. Plant Biol. 57:730-759 (2006)). We observed that treatment with exogenous ABA significantly decreased both the chlorophyll and Proto contents but stimulated ABAR expression and Mg-chelatase activity and enhanced the MgProto contents (FIG. 7a). This positive regulation of ABAR by ABA seems to support its function as an ABA sensor and indicates that ABA-induced chlorophyll decrease might not be attributable to the action of ABA on ABAR. However, as an ABA and Proto dual-ligand-binding protein, ABAR binds ABA independently of Proto (FIG. 8), indicating that ABA signal perception might be distinct from Proto binding.

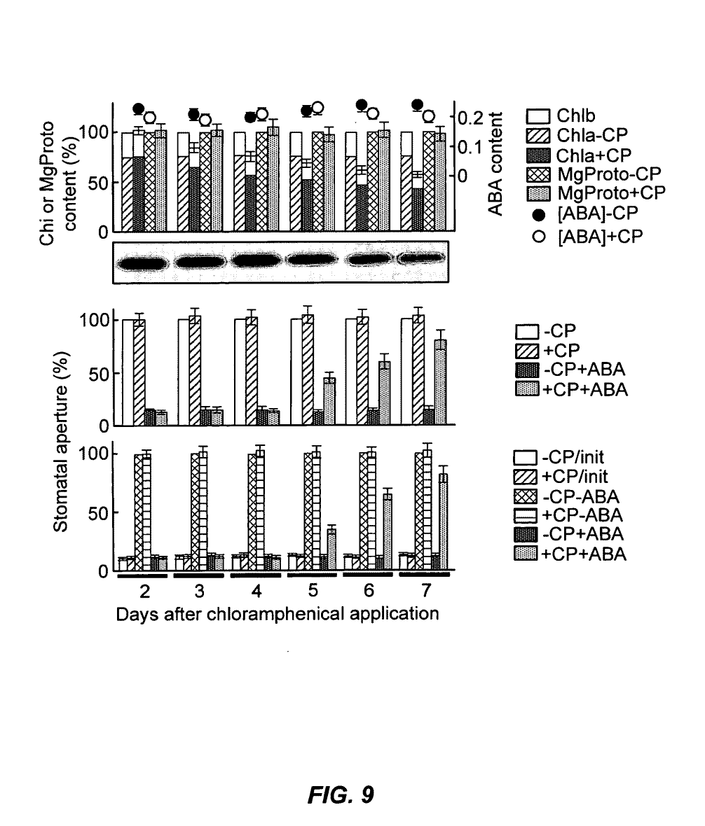

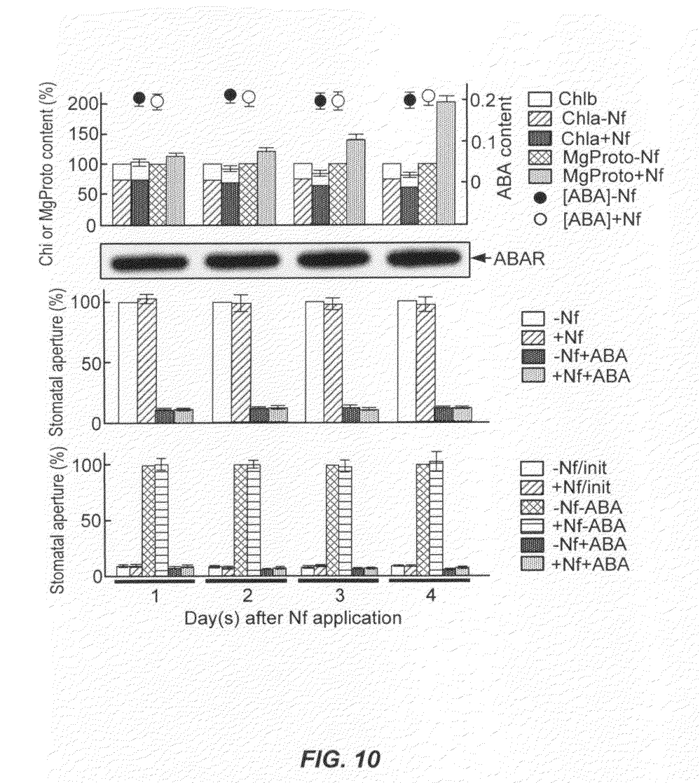

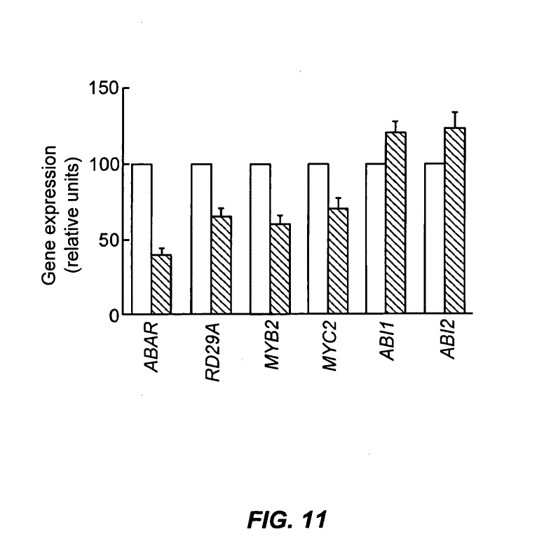

We observed, in a pharmaceutical assay using both norflurazon (Chamovitz, D. et al., Plant Mol. Biol. 16:967-974 (1991)) (an inhibitor of the carotenoid biosynthetic enzyme phytoene desaturase that causes photo-oxidative damage to chloroplasts) and chloramphenicol (CP; an inhibitor of plastid translation), that an ABA-insensitive stomatal movement occurred in parallel with a decrease in ABAR levels (FIG. 9), but no correlation of ABA-responsive stomatal movement with chlorophyll or MgProto contents was found (FIGS. 9, 10). We also used a chemical-regulated inducible RNAi system (Guo, H. S. et al., Plant J. 34:383-392 (2003)) to investigate ABAR-mediated ABA signalling. After induction by 17beta-oestradiol, a decrease in the ABAR levels was observed without an alteration in the chlorophyll and MgProto contents (FIG. 7b, and data not shown), and this decrease in ABAR levels induced a parallel insensitivity of stomatal movement to ABA (FIG. 7b). Using the protoplasts prepared from the inducible RNAi plants, we found that a decline of ABAR expression repressed the mRNA levels of RD29A (Yamaguchi-Shinozaki, K. et al., Plant Cell 6:251-264 (1994)), MYB2 and MYC2 (Abe, H. et al., Plant Cell 15:63-78 (2003))—genes that respond positively to ABA—but upregulated two negative regulators of ABA signalling, namely ABI1 (Leung, J. et al., Science 264:1448-1452 (1994); Meyer, K. et al., Science 264:1452-1455 (1994); Gosti, F. et al., Plant Cell 11:1897-1909 (1999)) and ABI2 (Gosti, F. et al., Plant Cell 11:1897-1909 (1999); Leung, J. et al., Plant Cell 9:759-771 (1997)) (FIG. 11). These approaches provide additional, more direct, evidence for functions of ABAR as a positive regulator of ABA signalling independently of chlorophyll and MgProto.

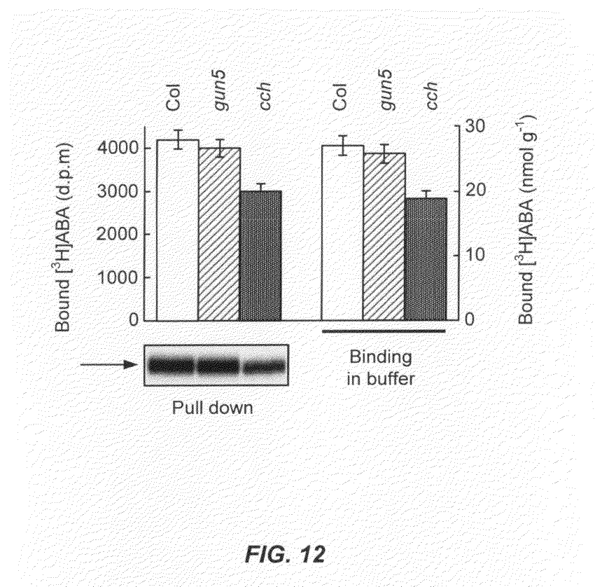

Further assays were performed with a series of mutants defective in chlorophyll metabolism or plastid signalling. hy1 (Davies, S. et al., Proc. Natl. Acad. Sci. USA 96:6541-6546 (1999); Muramoto, T. et al., Plant Cell 11:335-347 (1999)) and hy2 (Kohchi, T. et al., Plant Cell 13:425-436 (2001)) mutants, containing lesions in haem oxygenase and phytochromobilin synthase genes, respectively, are alleles of two gun mutants, gun2 and gun3 respectively, that are defective in plastid signaling (Mochizuki, N. et al., Proc. Natl. Acad. Sci. USA 98:2053-2058 (2001); Surpin, M., et al., Plant Cell S327-S338 (2002); Nott, A. et al., Annu. Rev. Plant Biol. 57:730-759 (2006)). The gun4 mutant has a lesion in a second Proto-binding protein-encoding gene GUN4 (Larkin, R. M. et al., Science 299:902-906 (2003); Verdecia, M. A. et al., Plos Biol. 3:777-789 (2005)). ch1 mutants contain lesions in the gene encoding chlorophyll a oxygenase (Espineda, C. E. et al., Proc. Natl. Acad. Sci. USA 96:10507-10511 (1999)). cch is also a gun mutant and an allele of gun5 but with a single nucleotide substitution at a different site, resulting in a single amino acid mutation Pro 642right arrowLeu (Mochizuki, N. et al., Proc. Natl. Acad. Sci. USA 98:2053-2058 (2001)). These mutants have lower chlorophyll contents except gun5, which possesses a chlorophyll level comparable to that of its wild type (Col , FIG. 7c), and they have ABAR levels comparable to those of their wild-type plants except the cch mutant, which has a lower level (FIG. 7c). All the gun mutants hy1/gun2, hy2/gun3, gun4, gun5 and cch have been shown to be involved in the same MgProto-triggered plastid-signalling pathway (Strand, A., et al., Nature 421:79-83 (2003)), but only the cch mutant had the ABA-insensitive phenotypes in germination (FIG. 7d), seedling growth (FIG. 7e) and ABA-induced stomatal movement (FIG. 7f). In all the mutants, no significant correlation of the chlorophyll levels with the ABA-responsive phenotypes was observed (FIG. 7c-f). The cch mutation, but not gun5, significantly decreased the ABA-binding activity of ABAR (FIG. 12), which may explain the ABA-insensitive phenotypes in the cch mutant and wild-type phenotypes in the gun5 mutant. Taken together, these data show clearly that ABAR is a positive regulator in ABA signal transduction involved in a signalling process that is distinct from chlorophyll metabolism and MgProto-mediated plastid retrograde signalling.

### ABAR is Ubiquitous and Regulates ABA-Signalling Genes

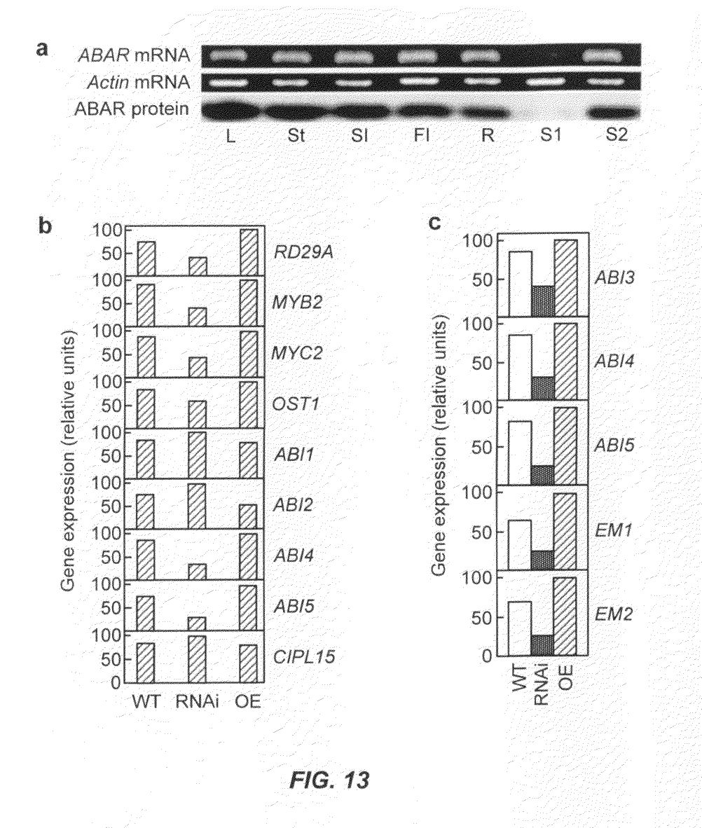

The CHLH expression was previously reported to be limited to the green tissues (Walker, C. J. et al., Biochem. J. 327:321-333 (1997); Surpin, M., et al., Plant Cell S327-S338 (2002)). Available data at the Genevestigator site (http://www.genevestigator.ethz.ch) show the presence of Arabidopsis CHLH mRNA in seeds. We found that ABAR/CHLH is a protein that is expressed ubiquitously in the non-green tissues, including the roots (FIG. 13a). ABAR might therefore function at the whole-plant level.

We found that downregulation of ABAR expression by RNAi decreased the levels of the positive regulators of ABA signalling RD29A (Yamaguchi-Shinozaki, K. et al., Plant Cell 6:251-264 (1994)), MYB2 (Abe, H. et al., Plant Cell 15:63-78 (2003)), MYC2 (Abe, H. et al., Plant Cell 15:63-78 (2003)), ABI4 (Finkelstein, R. R. Plant J. 5:765-771 (1994); Finkelstein, R. R. et al., Plant Cell 10:1043-1054 (1998)), ABI5 (Finkelstein, R. R. Plant J. 5:765-771 (1994); Finkelstein, R. R. et al., Plant Cell 12:599-609 (2000)) and OST1 (Mustilli, A. C. et al., Plant Cell 14:3089-3099 (2002)), ut enhanced the levels of three negative regulators, ABI1 (Leung, J. et al., Science 264:1448-1452 (1994); Meyer, K. et al., Science 264:1452-1455 (1994); Gosti, F. et al., Plant Cell 11:1897-1909 (1999)), ABI2 (Gosti, F. et al., Plant Cell 11:1897-1909 (1999) Leung, J. et al., Plant Cell 9:759-771 (1997)) and CIPK15 (Guo, Y. et al., Dev. Cell 3:233-244 (2002)) in leaves (FIG. 13b). These results are essentially consistent with those from the inducible RNAi protoplasts (FIG. 11). The seed-specific ABA-signalling genes ABI3 (Giraudat, J. et al., Plant Cell 4:1251-1261 (1992); Nambara, E. et al., Plant J 2:435-441 (1992)), ABI4 (Finkelstein, R. R. Plant J 5:765-771 (1994); Finkelstein, R. R. et al., Plant Cell 10:1043-1054 (1998)) and ABI5 (Finkelstein, R. R. Plant J. 5:765-771 (1994); Finkelstein, R. R. et al., Plant Cell 12:599-609 (2000)) and their downstream genes EM1 and EM6, which are both responsible for late embryogenesis (Finkelstein, R. R. et al., Plant Cell S15-S45 (2002); Manfre, A. J. et al., Plant Physiol. 140:140-140 (2006)), were all downregulated in the siliques of the RNAi plants (FIG. 13c). In most cases the ABAR-overexpressing plants regulated these genes in a manner contrary to the RNAi plants (FIG. 13b, c). The expression levels of these genes in the gun5 mutant were similar to those in the wild-type Columbia (data not shown). The regulation of these ABA-signalling genes by ABAR supports the contention that ABAR is a positive regulator and indicates that it might function through various pathways.

### Discussion

Previous studies have shown a multiplicity and complexity of ABA perception sites that may act at the outside or inside of cells to mediate different biological functions in plants (Leung, J. et al., Annu. Rev. Plant Physiol. Plant Mol. Biol. 49:199-222 (1998); Finkelstein, R. R. et al., Plant Cell S15-S45 (2002); Himmelbach, A. et al., Curr. Opin. Plant Biol. 6:470-479 (2003); Razem, F. A. et al., Nature 439:290-294 (2006)). We show that ABAR is an ABA receptor to perceive the ABA signal in seed germination, post-germination growth and stomatal movement, essentially from the following evidence: first, ABAR specifically binds ABA; second, transgenic downregulation of ABAR expression results in a decline in the number of ABA-binding sites and leads to ABA-insensitive phenotypes; third, ABAR-overexpressing plants have ABA-hypersensitive phenotypes with an elevated number of ABA-binding sites; fourth, a loss-of-function mutation in ABAR results in an immature embryo; and last, a cch mutant that downregulates both ABAR expression and ABA-binding activity is an ABA-insensitive mutant like the post-transcriptional gene-silencing RNAi or antisense mutants. Thus, ABAR is a common key component in the chlorophyll biosynthetic process of chelating Mg2+ to Proto, plastid retrograde signalling to the nucleus and perception of the ABA signal. However, ABAR-mediated ABA signalling is distinct from other pathways like ABAR/GUN5-mediated plastid-to-nucleus signaling, which is independent of chlorophyll biosynthesis (Mochizuki, N. et al., Proc. Natl. Acad. Sci. USA 98:2053-2058 (2001)).

As a receptor, ABAR regulates a series of the components involved in the ABA signalling network (FIG. 13), but the downstream components interacting directly with ABAR will have to be identified in the future to explain how ABA signal perception by ABAR is relayed in cells. ABAR is a single-copy gene in the Arabidopsis genome, is highly conserved in plant species and even shares high sequence similarities to its homologues in bacteria. This evolutionary conservation indicates a possibly vital role for it in these organisms. Gaining a further insight into how ABAR works in this complex signalling network will be of great interest in understanding cell signalling in plants.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of enhancing abscisic acid sensitivity in a plant, the method comprising introducing an recombinant expression cassette into a plant, wherein the expression cassette comprises a promoter operably linked to a polynucleotide encoding the H subunit of Mg-chelatase, wherein the promoter is heterologous to the polynucleotide, wherein the plant has increased abscisic acid sensitivity compared to an otherwise identical plant lacking the expression cassette.

2. The method of claim 1, wherein the plant has improved drought tolerance compared to an otherwise identical plant lacking the expression cassette

3. The method of claim 1, wherein the H subunit of Mg-chelatase is at least 80% identical to a protein selected from the group consisting of SEQ ID NO:2, SEQ ID NO:4, SEQ ID NO:6, SEQ ID NO:8, SEQ ID NO:10 and SEQ ID NO:12.

4. The method of claim 1, wherein the promoter is constitutive.

5. The method of claim 1, wherein the promoter is inducible.

6. The method of claim 1, wherein the promoter is tissue-specific.

7. The method of claim 1, wherein the promoter directs expression in guard cells.

8. The method of claim 1, comprising generating a plurality of plants comprising the introduced expression cassette, and screening the plants for abscisic acid sensitivity compared to an otherwise identical plant lacking the expression cassette.

9. A method of decreasing abscisic acid sensitivity in a plant, the method comprising introducing an recombinant expression cassette into a plant, wherein the expression cassette comprises a promoter operably linked to a polynucleotide comprising at least 20 nucleotides complementary or identical to a contiguous sequence in a cDNA encoding an endogenous H subunit of Mg-chelatase in the plant, wherein the promoter is heterologous to the polynucleotide, thereby reducing expression of the H subunit Mg-chelatase in the plant, wherein the plant has reduced abscisic acid sensitivity compared to an otherwise identical plant lacking the expression cassette.

10. The method of claim 9, wherein the polynucleotide comprises at least 50 nucleotides complementary or identical to a contiguous sequence in a cDNA encoding a H subunit of Mg-chelatase in the plant.

11. The method of claim 9, wherein the polynucleotide comprises at least 200 nucleotides complementary or identical to a contiguous sequence in a cDNA encoding a H subunit of Mg-chelatase in the plant.

12. The method of claim 9, wherein the H subunit of Mg-chelatase is at least 80% identical to a protein selected from the group consisting of SEQ ID NO:2, SEQ ID NO:4, SEQ ID NO:6, SEQ ID NO:8, SEQ ID NO:10 and SEQ ID NO:12.

13. The method of claim 9, wherein the promoter directs expression of the polynucleotide to abscission zones of the plant.

14. A recombinant expression cassette comprising a promoter operably linked to a polynucleotide encoding the H subunit of Mg-chelatase, wherein the promoter is heterologous to the polynucleotide, and wherein introduction of the expression cassette into a plant results in enhanced abscisic acid sensitivity in the plant compared to an otherwise identical plant lacking the expression cassette.

15. The recombinant expression cassette of claim 14, wherein introduction of the expression cassette into a plant results in improved drought tolerance in the plant compared to an otherwise identical plant lacking the expression cassette

16. The recombinant expression cassette of claim 14, wherein the H subunit of Mg-chelatase is at least 80% identical to a protein selected from the group consisting of SEQ ID NO:2, SEQ ID NO:4, SEQ ID NO:6, SEQ ID NO:8, SEQ ID NO:10 and SEQ ID NO:12.

17. The recombinant expression cassette of claim 14, wherein the promoter is constitutive.

18. The recombinant expression cassette of claim 14, wherein the promoter is inducible.

19. The recombinant expression cassette of claim 14, wherein the promoter is tissue-specific.

20. The recombinant expression cassette of claim 14, wherein the promoter directs expression in guard cells.

21. A transgenic plant comprising a recombinant expression cassette, wherein the expression cassette comprises a promoter operably linked to a polynucleotide encoding the H subunit of Mg-chelatase, wherein the promoter is heterologous to the polynucleotide, and wherein the plant has enhanced abscisic acid sensitivity compared to an otherwise identical plant lacking the expression cassette.

22. The plant of claim 21, wherein the plant has improved drought tolerance compared to an otherwise identical plant lacking the expression cassette

23. The plant of claim 21, wherein the H subunit of Mg-chelatase is at least 80% identical to a protein selected from the group consisting of SEQ ID NO:2, SEQ ID NO:4, SEQ ID NO:6, SEQ ID NO:8, SEQ ID NO:10 and SEQ ID NO:12.

24. The plant of claim 21, wherein the promoter is constitutive.

25. The plant of claim 21, wherein the promoter is inducible.

26. The plant of claim 21, wherein the promoter is tissue-specific.

27. The plant of claim 21, wherein the promoter directs expression in guard cells.

28. A seed, flower, leaf or fruit from the plant of claim 21.


SEQUENCE LISTING (60 sequences)

SEQ ID NO: 1 - DNA, 4428 bp, Arabidopsis thaliana
  thale cress magnesium-protoporphyrin IX chelatase(Mg-chelatase) H subunit (CHLH), abscisic acid receptor (ABAR)
gcggccgccgatgagagaatcataaactcccacttggagctcaaaaagtgtaagagacaaccaacaataaacgattcatctcttctcctatcctctcctcttcgaattcaacgtttggagaatccagcagccgcaaaatggcttcgcttgtgtattctccattcactctatccacttctaaagcagagcatctctcttcgctcactaacagtaccaaacattctttcctccggaagaaacacagatcaaccaaaccagccaaatctttcttcaaggtgaaatctgctgtatctggaaacggcctcttcacacagacgaacccggaggtgcgtcgtatagttccgatcaagagagacaacgttccgacggtgaaaatcgtctacgtcgtcctcgaggctcagtaccagtcttctctcagtgaagccgtgcaatctctcaacaagacttcgagattcgcatcctacgaagtggttggatacttggtcgaggagcttagagacaagaacacttacaacaacttctgcgaagaccttaaagacgccaacatcttcattggttctctgatcttcgtcgaggaattggcgattaaagttaaggatgcggtggagaaggagagagacaggatggacgcagttcttgtcttcccttcaatgcctgaggtaatgagactgaacaagcttggatcttttagtatgtctcaattgggtcagtcaaagtctccgtttttccaactcttcaagaggaagaaacaaggctctgctggttttgccgatagtatgttgaagcttgttaggactttgcctaaggttttgaagtacttacctagtgacaaggctcaagatgctcgtctctacatcttgagtttacagttttggcttggaggctctcctgataatcttcagaattttgttaagatgatttctggatcttatgttccggctttgaaaggtgtcaaaatcgagtattcggatccggttttgttcttggatactggaatttggcatccacttgctccaaccatgtacgatgatgtgaaggagtactggaactggtatgacactagaagggacaccaatgactcactcaagaggaaagatgcaacggttgtcggtttagtcttgcagaggagtcacattgtcactggtgatgatagtcactatgtggctgttatcatggagcttgaggctagaggtgctaaggtcgttcctatattcgcaggagggttggatttctctggtccagtagagaaatatttcgtagacccggtgtcgaaacagcccatcgtaaactctgctgtctccttgactggttttgctcttgttggtggacctgcaaggcaggatcatcccagggctatcgaagccctgaaaaagctcgatgttccttaccttgtggcagtaccactggtgttccagacgacagaggaatggctaaagagcacacttggtctgcatcccatccaggtggctctgcaggttgccctccctgagcttgatggagcgatggagccaatcgttttcgctggtcgtgaccctagaacagggaagtcacatgctctccacaagagagtggagcaactctgcatcagagcgattcgatggggtgagctcaaaagaaaaactaaggcagagaagaagctggcaatcactgttttcagtttcccacctgataaaggtaatgtagggactgcagcttacctcaatgtgtttgcttccatcttctcggtgttaagagacctcaagagagatggctacaatgttgaaggccttcctgagaatgcagagactcttattgaagaaatcattcatgacaaggaggctcagttcagcagccctaacctcaatgtagcttacaaaatgggagtccgtgagtaccaagacctcactccttatgcaaatcccctggaagaaaactgggggaaacctccggggaaccttaactcagatggagagaaccttcttgtctatggaaaagcgtacggtaatgttttcatcggagtgcaaccaacatttgggtatgaaggtgatcccatgaggctgcttttctccaagtcagcaagtcctcatcacggttttgctgcttactactcttatgtagaaaagatcttcaaagctgatgctgttcttcattttggaacacatggttctctcgagtttatgcccgggaagcaagtgggaatgagtgatgcttgttttcccgacagtcttatcgggaacattcccaatgtctactattatgcagctaacaatccctctgaagctaccattgcaaagaggagaagttatgccaacaccatcagttatttgactcctccagctgagaatgctggtctatacaaagggctgaagcaggtgagtgagctgatatcgtcctatcagtctctgaaggacacggggagaggtccacagatcgtcagttccatcatcagcacagctaagcaatgtaatcttgataaggatgtggatcttccagatgaaggcttggagttgtcacctaaagacagagattctgtggttgggaaagtttattccaagattatggagattgaatcaaggcttttgccgtgcgggcttcacgtcattggagagcctccatccgccatggaagctgtggccacactggtcaacattgctgctctagatcgtccggaggatgagatttcagctcttccttctatattagctgagtgtgttggaagggagatagaggatgtttacagaggaagcgacaagggtatcttgagcgatgtagagcttctcaaagagatcactgatgcctcacgtggcgctgtttccgcctttgtggaaaaaacaacaaatagcaaaggacaggtggtggatgtgtctgacaagcttacctcgcttcttgggtttggaatcaatgagccatgggttgagtatttgtccaacaccaagttctacagggcgaacagagataagctcagaacagtgtttggtttccttggagagtgcctgaagttggtggtcatggacaacgaactagggagtctaatgcaagctttggaaggcaagtacgtcgagcctggccccggaggtgatcccatcagaaacccaaaggtcttaccaaccggaaaaacatccattgccttagatcctcaggctattcccacaacagcagcaatggcaagtgccaagattgtggttgagaggttggtagagagacagaagctcgaaaacgaagggaaatatcccgagacaatcgcgcttgttctttggggaactgacaacatcaaaacatatggggagtctcttgggcaggttctttggatgattggtgtgagaccaattgctgatacttttggaagagtgaaccgtgtcgagcctgtgagcttagaagaactaggaaggccgaggatcgatgtagttgttaactgctcaggggtcttccgtgatctctttatcaaccagatgaaccttcttgaccgagctatcaagatggtggcggacgtagatgagcccgtagagcaaaattttgtaaggaaacacgcgttggaacaagcagaggcgcttggcattgatattagagaggcagcgacaagagttttctcaaacgcttcagggtcatactcagccaacatcagtcttgctgttgaaaactcgtcatggaacgatgagaaacagcttcaggacatgtacttgagccgcaaatcgtttgcgtttgatagtgatgctcctggagcaggaatggctgagaagaagcaggtctttgagatggctcttagcactgcagaagtcaccttccagaacctggattcttcagagatttctttgactgatgtgagccactacttcgattctgaccctacaaatctagttcagagtttgaggaaggataagaagaaaccaagctcttacattgctgacactacaactgcaaacgcgcaggtgaggacactatctgagacagtgaggctggacgcaagaacaaagctgctgaatccaaagtggtacgaaggaatgatgtcaagtggatatgaaggagttcgtgagatagagaagagactgtccaacactgtgggatggagtgcaacgtcaggtcaagtagacaattgggtctacgaggaggccaactcaactttcatccaagacgaggagatgctgaaccgtctcatgaacaccaatcccaactccttcaggaaaatgcttcagactttcttggaggccaatggtcgtggctactgggacacttccgctgaaaacatagagaagctcaaggaattgtactcgcaggtggaagacaagatcgaagggatcgatcgataaacaatgggatataagcctttcttcttgtaaatgaacttaaaactttatttttgtgtattacagtattctaccaaacgacatcatctctctgtgaataagagaatgttttggctgacattttctgactttaaaaaaaaaaaaaaaa

SEQ ID NO: 2 - protein, 1381 aa, Arabidopsis thaliana
  thale cress magnesium-protoporphyrin IX chelatase(Mg-chelatase) H subunit (CHLH), abscisic acid receptor (ABAR)
MASLVYSPFTLSTSKAEHLSSLTNSTKHSFLRKKHRSTKPAKSFFKVKSAVSGNGLFTQTNPEVRRIVPIKRDNVPTVKIVYVVLEAQYQSSLSEAVQSLNKTSRFASYEVVGYLVEELRDKNTYNNFCEDLKDANIFIGSLIFVEELAIKVKDAVEKERDRMDAVLVFPSMPEVMRLNKLGSFSMSQLGQSKSPFFQLFKRKKQGSAGFADSMLKLVRTLPKVLKYLPSDKAQDARLYILSLQFWLGGSPDNLQNFVKMISGSYVPALKGVKIEYSDPVLFLDTGIWHPLAPTMYDDVKEYWNWYDTRRDTNDSLKRKDATVVGLVLQRSHIVTGDDSHYVAVIMELEARGAKVVPIFAGGLDFSGPVEKYFVDPVSKQPIVNSAVSLTGFALVGGPARQDHPRAIEALKKLDVPYLVAVPLVFQTTEEWLKSTLGLHPIQVALQVALPELDGAMEPIVFAGRDPRTGKSHALHKRVEQLCIRAIRWGELKRKTKAEKKLAITVFSFPPDKGNVGTAAYLNVFASIFSVLRDLKRDGYNVEGLPENAETLIEEIIHDKEAQFSSPNLNVAYKMGVREYQDLTPYANPLEENWGKPPGNLNSDGENLLVYGKAYGNVFIGVQPTFGYEGDPMRLLFSKSASPHHGFAAYYSYVEKIFKADAVLHFGTHGSLEFMPGKQVGMSDACFPDSLIGNIPNVYYYAANNPSEATIAKRRSYANTISYLTPPAENAGLYKGLKQVSELISSYQSLKDTGRGPQIVSSIISTAKQCNLDKDVDLPDEGLELSPKDRDSVVGKVYSKIMEIESRLLPCGLHVIGEPPSAMEAVATLVNIAALDRPEDEISALPSILAECVGREIEDVYRGSDKGILSDVELLKEITDASRGAVSAFVEKTTNSKGQVVDVSDKLTSLLGFGINEPWVEYLSNTKFYRANRDKLRTVFGFLGECLKLVVMDNELGSLMQALEGKYVEPGPGGDPIRNPKVLPTGKTSIALDPQAIPTTAAMASAKIVVERLVERQKLENEGKYPETIALVLWGTDNIKTYGESLGQVLWMIGVRPIADTFGRVNRVEPVSLEELGRPRIDVVVNCSGVFRDLFINQMNLLDRAIKMVADVDEPVEQNFVRKHALEQAEALGIDIREAATRVFSNASGSYSANISLAVENSSWNDEKQLQDMYLSRKSFAFDSDAPGAGMAEKKQVFEMALSTAEVTFQNLDSSEISLTDVSHYFDSDPTNLVQSLRKDKKKPSSYIADTTTANAQVRTLSETVRLDARTKLLNPKWYEGMMSSGYEGVREIEKRLSNTVGWSATSGQVDNWVYEEANSTFIQDEEMLNRLMNTNPNSFRKMLQTFLEANGRGYWDTSAENIEKLKELYSQVEDKIEGIDR

SEQ ID NO: 3 - DNA, 4164 bp, Oryza sativa
  rice (japonica cultivar-group) magnesium-protoporphyrin IX chelatase (Mg-chelatase) H subunit (CHLH), abscisic acid receptor (ABAR)
atgtcgtctttagtgtcgacgccgttcacgacggcaaccggggtgcagaagaagctgggggcgcccgtgccgctccactcgttcttgctgagccggcggcagccagcggcgggcgcggggaggggacgcgcggcggcggcggcgatacggtgcgcggtggccgggaacgggctgttcacgcagaccaagccggaggtgcggcgcgtggtgccgcccgagggcgacgcgtcgcggcgcggggtgccgcgggtgaaggtggtgtacgtcgtgctggaggcgcagtaccagtcgtcggtgacggcggcggtgcgggagctcaacgccgacccgcgccgcgcggcggggttcgaggtggtgggctacctcgtggaggagctccgggacgaggagacgtacaagaccttctgcgccgacctcgccgacgccaatgtgttcatcggctcgctcatcttcgtggaggagctcgcgctcaaggtgaaggatgccgtcgagaaggagcgcgaccggatggacgccgtgctcgtcttcccgtcgatgccggaggtcatgcgcctcaacaagctcggctccttcagcatgtcccagctcggccagtccaagagcccattcttccagctcttcaagcgcaagaagaactccggtggcttcgccgatagcatgctcaagctggtgcgcacgctgcccaaggtgctcaagtacttgccctccgacaaggcgcaggacgcccggctgtacatcctcagcctccagttctggctcggaggctcgccggacaacctccagaatttcctcaagatgatcgccgtctcctacgtgccggcgctcaagggcgccgacatcaagtacgacgaccccgtcctcttcctcgacgctggtatctggcacccgctggcgcccaccatgtacgacgacgtcaaggagtacctcaactggtacggcacccgccgcgacaccaacgacaagctcaaggaccccaacgcgccggtgatcggcctcgttttgcagaggagccacattgtcaccggagacgacggtcactacgtcgccgtgatcatggagctggaggccaagggtgccaaggtcataccgatcttcgccggcgggctggacttctcgggacccacgcagcggtacctcgtcgacccgatcaccggaaagccgttcgtgaacgcggtggtgtcgctcaccgggttcgcgctcgtcggagggccagcgaggcaggaccatcccaaggcgatcgccgcgctgcagaagctcgacgtgccgtacatcgtggcactgccgctcgtgttccagacgacagaggagtggctgaacagcacattgggcctgcacccgattcaggtggcgctgcaggttgcgctcccggagcttgacggtggcatggagcccattgtgttcgccggccgtgaccccagaacagggaagtcacatgcgttgcacaagagggtggagcagctctgcactagagcaatcagatgggcagagctgaagaggaaaactaaggaggagaagaaactggcaatcactgttttcagcttcccaccagacaaaggcaatgttggcacagcagcatacctgaatgttttcaactccatctactccgtcctccaagatctgaagaaggatggctacaatgttgagggtcttccagacacagctgaggccctcatcgaggaggttattcatgataaggaggcccaattcaatagccccaacctcaatgttgcttaccgcatgaacgtgcgggagtaccagtcactcacttcctatgcctccttgctggaggagaactggggcaagccacctgggaaccttaattctgatggtgaaaacctccttgtctatgggaaacagtacggcaatgtattcattggagttcagcccacttttggctatgaaggagatccgatgcggcttctgttctcaaaatctgctagccctcaccatggctttgcagcatactacacctttgttgagaagatcttccaggctgatgctgttcttcactttggtacccatgggtctcttgagttcatgccagggaagcaggttgggatgagtgatgcatgctatcctgacagtctcattggcaacatccccaatatctactactatgcagcaaacaatccatcagaagcaactgttgccaagcgcagaagctatgcaaacaccataagctacctgacaccaccagctgaaaatgctggtctctacaaggggctcaagcagctttcagagctcatctcttcttaccaatctctcaaggacacaggacgtggtccgcagattgtgagctcaatcattagcactgcaaaacagtgtaatcttgacaaggatgttcccttgcctgaggaaggtgtggagcttccaccaaatgagcgtgaccttattgttggaaaggtgtatgccaagatcatggaaatagaatcacgcctcctaccatgcggtctgcatgtgataggtgagccaccaagtgccatcgaggctgtggccaccttggtgaacatagcttcccttgatcgcccagaggatgaaatatactcactgcctaacatacttgctcagacagtgggcaggaacattgaagatgtgtacagaggaagtgacaagggaatactggcggatgttgaactgttgaggcagataacagaagcttcacgtggtgccatcactacctttgttgagaggactacaaacaacaaagggcaagttgttgatgttacaaacaaacttagtaccatgcttggttttggtttatcagaaccatgggtacaacacttgtccaagaccaagttcatcagagcagacagagagaaattgagaaccttgtttactttcttgggagaatgcttgaagctaattgtggcagataatgagctgggaagcttgaaacttgccctcgagggaagctatgttgaacctggccctggtggtgatccaatccgtaacccgaaggttctcccgacagggaagaacatccatgctcttgaccctcaggcaatcccaactacagctgccttgaagagcgccaaaattattgtagaccgtctgctggagcggcaaaaggttgacaatggtggcaagtatcctgagacaattgcacttgtcttgtggggcaccgataacatcaagacctatggtgagtcattggcccaggtgctgtggatgattggtgtgcgcccggttgctgacacctttggccgtgtcaaccgtgtggaacctgtcagccttgaggagcttggacgtcccaggattgacgttgttatcaactgctcgggtgtcttcagagatcttttcatcaaccagatgaatctactggaccgggcagtgaagatggttgccgaactggatgagccagaagagatgaactacgtgcgtaagcatgcacaagagcaggcacgggaacttggcgtttcattaagagaggcggcaacaagggtgttctcaaatgcatcaggctcttactcatcgaatgtgaacttggcagtggagaatgcatcatggactgatgagaagcagctccaggacatgtacctgagtcgcaagtcttttgcatttgattgtgatgctccaggggcaggcatgcgagagcaacgcaagacatttgagcttgctctagcaacagcagatgccacattccagaacctagactcatcagagatttcactaacagatgtgagccactactttgactcagacccgacaaagctggtgcaaggactgcgcaaggatgggcgggcaccttcctcatacatagcagatacaaccacagcaaatgcacaggtgaggacattgtcagagacagtgcgccttgatgcaaggacaaagctactgaaccctaagtggtacgaggggatgatgaaaagtggctacgagggagttagagagattgagaagcggctgacaaatactgttggatggagtgcaacatctggacaggttgacaactgggtttatgaggaggcaaatgccacatttattgaagatgaggctatgaggaagaggctcatggacaccaaccccaattcattcaggaagctagttcagaccttcctagaagccagtggcagaggctactgggagacatcagaggaaaacttggaaaagctcagggagctctactctgaggttgaagacaagattgaaggaattgaccggtaa

SEQ ID NO: 4 - protein, 1387 aa, Oryza sativa
  rice (japonica cultivar-group) magnesium-protoporphyrin IX chelatase (Mg-chelatase) H subunit (CHLH), abscisic acid receptor (ABAR)
MSSLVSTPFTTATGVQKKLGAPVPLHSFLLSRRQPAAGAGRGRAAAAAIRCAVAGNGLFTQTKPEVRRVVPPEGDASRRGVPRVKVVYVVLEAQYQSSVTAAVRELNADPRRAAGFEVVGYLVEELRDEETYKTFCADLADANVFIGSLIFVEELALKVKDAVEKERDRMDAVLVFPSMPEVMRLNKLGSFSMSQLGQSKSPFFQLFKRKKNSGGFADSMLKLVRTLPKVLKYLPSDKAQDARLYILSLQFWLGGSPDNLQNFLKMIAVSYVPALKGADIKYDDPVLFLDAGIWHPLAPTMYDDVKEYLNWYGTRRDTNDKLKDPNAPVIGLVLQRSHIVTGDDGHYVAVIMELEAKGAKVIPIFAGGLDFSGPTQRYLVDPITGKPFVNAVVSLTGFALVGGPARQDHPKAIAALQKLDVPYIVALPLVFQTTEEWLNSTLGLHPIQVALQVALPELDGGMEPIVFAGRDPRTGKSHALHKRVEQLCTRAIRWAELKRKTKEEKKLAITVFSFPPDKGNVGTAAYLNVFNSIYSVLQDLKKDGYNVEGLPDTAEALIEEVIHDKEAQFNSPNLNVAYRMNVREYQSLTSYASLLEENWGKPPGNLNSDGENLLVYGKQYGNVFIGVQPTFGYEGDPMRLLFSKSASPHHGFAAYYTFVEKIFQADAVLHFGTHGSLEFMPGKQVGMSDACYPDSLIGNIPNIYYYAANNPSEATVAKRRSYANTISYLTPPAENAGLYKGLKQLSELISSYQSLKDTGRGPQIVSSIISTAKQCNLDKDVPLPEEGVELPPNERDLIVGKVYAKIMEIESRLLPCGLHVIGEPPSAIEAVATLVNIASLDRPEDEIYSLPNILAQTVGRNIEDVYRGSDKGILADVELLRQITEASRGAITTFVERTTNNKGQVVDVTNKLSTMLGFGLSEPWVQHLSKTKFIRADREKLRTLFTFLGECLKLIVADNELGSLKLALEGSYVEPGPGGDPIRNPKVLPTGKNIHALDPQAIPTTAALKSAKIIVDRLLERQKVDNGGKYPETIALVLWGTDNIKTYGESLAQVLWMIGVRPVADTFGRVNRVEPVSLEELGRPRIDVVINCSGVFRDLFINQMNLLDRAVKMVAELDEPEEMNYVRKHAQEQARELGVSLREAATRVFSNASGSYSSNVNLAVENASWTDEKQLQDMYLSRKSFAFDCDAPGAGMREQRKTFELALATADATFQNLDSSEISLTDVSHYFDSDPTKLVQGLRKDGRAPSSYIADTTTANAQVRTLSETVRLDARTKLLNPKWYEGMMKSGYEGVREIEKRLTNTVGWSATSGQVDNWVYEEANATFIEDEAMRKRLMDTNPNSFRKLVQTFLEASGRGYWETSEENLEKLRELYSEVEDKIEGIDR

SEQ ID NO: 5 - DNA, 4449 bp, Glycine max
  soybean magnesium-protoporphyrin IX chelatase (Mg-chelatase) H subunit (CHLH), abscisic acid receptor (ABAR)
ctatggaccccccaagttttcctttcaaagcaattttgtgctttgctttgattgtattccttttccttccctacacttcaatttgtagcaagaggaatttgttgtttcctacttagcttgattatttatcaatggcttctttggtatcttcaccatttactcttccaagctctaaacctgaccagcttcattctcttgcccagaagcatctttatcttcattctttccttcccaagaaagccaattacaatggtagctcaaaatcctctctgagagtgaaatgtgctgtcattggcaatggcctattcacccaaaccacccaagaagttcgtagaatagttccagagaatgaccaaaacttgccaacagttaaaattgtgtatgtggtccttgaggctcagtaccaatcatccatcactgctgcagtgatagctctcaacagcaaaaggaagcatgcttcctttgaggttgtgggttacttggttgaggagcttcgtgacgcggcgacgtacaagaccttctgcaaggacttggaggatgctaacatcttcattggttccttgatttttgtggaggagctcgccctcaagatcaaggctgcagtggagaaagaaagggacaggcttgatgcagttttggtgttcccatcaatgcctgaagtgatgaggctcaacaagttgggttctttcagcatgtcacagcttgggcagtctaagagcccctttttccagctgttcaagagaaagaagcctcagtctgctggctttgctgatagcatgttgaagcttgtgaggacattgccaaaggttttgaagtatttgccaagtgataaagctcaggatgccaggctctacatactgagtctgcagttttggcttggggggtctcctgataacttgcagaatttcctgaaaatgatttctggatcgtatattccggcgctgaaagggacgaagatcgagtattcggagccggttttgtacttggatgtggggatttggcaccctttggctccttgtatgtatgatgatgtcaaggagtatttgaattggtatggaaccagaagggatgcaaatgagaagctgaagagtccgaatgcaccggtcattggtttggttttgcagaggagtcatattgtgactggtgatgatgggcactatgtggctgtgatcatggagctggaggctagaggggctaaggtcattcccatttttgccggcggacttgacttttcagggccagtggagaagttcttcattgatccgattacaaagaaaccgtttgtgaattctgtggtttccctcactggctttgctcttgttggaggccctgcaaggcaggaccatccaagggcggtcgaggctttgatgaaacttgatgttccttacattgttgccctgccactggtgttccagacaacagaagaatggctcaacagtactcttggtctgcatccaattcaggttgctcttcaagttgctctgccagagctagatggaggcatggagccaattgttttcgctggtcgagatcctaaaacaggaaaatctcatgctcttcataagagagtggaacagctctgcatcagggcaatcagatgggctgaattgaaaagaaaatcaaaggaagagaagaaactagcaatcactgtcttcagtttccctccagacaaaggaaacgtgggaactgctgcctatctcaatgtcttcgcctccatatactcagttatgaaagaactaaaaaaagatggttacaatgttgatggccttccggagacttcagaagctttgattgaagatgtacttcatgacaaagaagcccaattcagcagcccaaatctgaacattgcttacaaaatgaatgttcgcgagtaccaaaatttgactccctatgccacagcactggaggagaattggggaaaaccccctggcaatctgaatgcagatggagaaaatcttctggtatatgggaaacagtatggtaatgtcttcataggtgttcagcctacatttggctatgaaggggatccaatgaggctgcttttctccaaatctgcaagccctcatcatggatttgcagcatattattcctttgttgagaaaatcttcaaagctgatgctgtacttcattttgggacacatggttcccttgaattcatgcctgggaaacaggtggggatgagtgatgtatgttaccctgacagtctgattgggaatattccaaatgtatattactatgctgcaaacaacccttctgaggccaccattgccaagcgcaggagttatgcaaacaccattagctatctgactcctccagcggaaaatgctgggctatacaaaggtcttaagcagttaagtgagctcatctcctcatatcagtccctcaaagacaccggccgtggggcacaaattgtgagttcaattatcagcacagctaaacaatgcaatcttgacaaggatgtgactctgccagacgagggtgaggagatcccacctaaagagcgcgaccttgtggttggacaggtgtattccaagatcatggagatcgagtctcgtttgttaccttgtgggcttcatattattggtgagcctccctcagccttggaagcagttgctacactggtcaacattgctgcacttgatcgtcctgaagatggtatttcttctcttccatcaatattagctgacactgtaggaagagatatagaagatgtgtatagaggaagtaacaaaggaatattgaaggatgtagagcttcttagacaaataaccgaggcatcacgtggagcaatcactgcctttgtggagcgcactactaataataagggtcaagttgttgatgtagctgataagcttagttcaatccttggatttggcataaatgagccatggatacagtacttatcaaacaccaaattttaccgagctgatagggaaaaacttagaaccttgtttgtgttcctgggagaatgtttgaagttgattgtggctgataatgaagtgggaagtttaaaacaagccttggaaggtaaatatgtggagccagggcctggtggtgacccaataagaaatccaaaagtcttgccaacaggaaagaatattcatgccctggaccctcaatctattcctacaactgcagcgatgcagagtgccaaaatagtggtagataggttgattgagaggcagaaagctgagaatgggggaaaatatcctgagacaattgcacttgtattgtggggaactgataatattaaaacatatggtgaatccctggctcaagtcttgtggatgattggtgtggaaccagtggctgatacctttggcagggtaaaccgggtggaacctgtaagtcttgaagagcttggaaggcctaggattgatgttgttgttaattgctcaggagtgtttagagaccttttcatcaatcagatgaatcttctggatagagcagtgaagatggttgctgaattagatgaaccagcagagcaaaactatgtaaaaaagcatgcatcagaacaagctcaagcccttggagttgaagttcgagaggcagcaacaaggatcttctccaatgcttctggctcctactcctcaaacataaacttggctgtggagaattcttcatggaatgatgagaagcagctccaggacatgtatctaagcagaaagtcttttgcttttgattctgatgcccctggtgctggcatgactgagaaaagaaaagtcttcgagatggctctgagcacagcagatgccacattccaaaatcttgattcatcagaaatttccctcactgacgtcagtcattactttgactcagacccgactaatctggtacaaaatctaaggaaagatgggaagaagcctagtgcatacattgctgatacaaccacagccaatgctcaggtacgtacactttctgagacggttagacttgacgcaagaaccaagctgttgaatccaaagtggtatgaaggcatgttgtctactggatatgagggtgtacgcgagatcgagaagagactcaccaatacagtggggtggagtgcaacttcaggccaagttgataactgggtgtatgaagaagccaacacaactttcattcaagatgagcaaatgctgaacaagctcatgagcactaatccaaactccttcaggaaactggtgcagacattcttggaagccaatggacgtggttattgggaaacttcagaagataatattgagaagctcaggcagttgtattcagaagtggaagacaaaattgaaggcattgatcgctgaagttaatagctattattgaaaagagcacacaacagtgaagagaagagcatcctttaaatgtgatttttgttttccttttctcattttttgagatcaagtctcctttgtgcagttctgaactccaaccccggtgaacaaataaaggttcaagttcataattgattta

SEQ ID NO: 6 - protein, 1383 aa, Glycine max
  soybean magnesium-protoporphyrin IX chelatase (Mg-chelatase) H subunit (CHLH), abscisic acid receptor (ABAR)
MASLVSSPFTLPSSKPDQLHSLAQKHLYLHSFLPKKANYNGSSKSSLRVKCAVIGNGLFTQTTQEVRRIVPENDQNLPTVKIVYVVLEAQYQSSITAAVIALNSKRKHASFEVVGYLVEELRDAATYKTFCKDLEDANIFIGSLIFVEELALKIKAAVEKERDRLDAVLVFPSMPEVMRLNKLGSFSMSQLGQSKSPFFQLFKRKKPQSAGFADSMLKLVRTLPKVLKYLPSDKAQDARLYILSLQFWLGGSPDNLQNFLKMISGSYIPALKGTKIEYSEPVLYLDVGIWHPLAPCMYDDVKEYLNWYGTRRDANEKLKSPNAPVIGLVLQRSHIVTGDDGHYVAVIMELEARGAKVIPIFAGGLDFSGPVEKFFIDPITKKPFVNSVVSLTGFALVGGPARQDHPRAVEALMKLDVPYIVALPLVFQTTEEWLNSTLGLHPIQVALQVALPELDGGMEPIVFAGRDPKTGKSHALHKRVEQLCIRAIRWAELKRKSKEEKKLAITVFSFPPDKGNVGTAAYLNVFASIYSVMKELKKDGYNVDGLPETSEALIEDVLHDKEAQFSSPNLNIAYKMNVREYQNLTPYATALEENWGKPPGNLNADGENLLVYGKQYGNVFIGVQPTFGYEGDPMRLLFSKSASPHHGFAAYYSFVEKIFKADAVLHFGTHGSLEFMPGKQVGMSDVCYPDSLIGNIPNVYYYAANNPSEATIAKRRSYANTISYLTPPAENAGLYKGLKQLSELISSYQSLKDTGRGAQIVSSIISTAKQCNLDKDVTLPDEGEEIPPKERDLVVGQVYSKIMEIESRLLPCGLHIIGEPPSALEAVATLVNIAALDRPEDGISSLPSILADTVGRDIEDVYRGSNKGILKDVELLRQITEASRGAITAFVERTTNNKGQVVDVADKLSSILGFGINEPWIQYLSNTKFYRADREKLRTLFVFLGECLKLIVADNEVGSLKQALEGKYVEPGPGGDPIRNPKVLPTGKNIHALDPQSIPTTAAMQSAKIVVDRLIERQKAENGGKYPETIALVLWGTDNIKTYGESLAQVLWMIGVEPVADTFGRVNRVEPVSLEELGRPRIDVVVNCSGVFRDLFINQMNLLDRAVKMVAELDEPAEQNYVKKHASEQAQALGVEVREAATRIFSNASGSYSSNINLAVENSSWNDEKQLQDMYLSRKSFAFDSDAPGAGMTEKRKVFEMALSTADATFQNLDSSEISLTDVSHYFDSDPTNLVQNLRKDGKKPSAYIADTTTANAQVRTLSETVRLDARTKLLNPKWYEGMLSTGYEGVREIEKRLTNTVGWSATSGQVDNWVYEEANTTFIQDEQMLNKLMSTNPNSFRKLVQTFLEANGRGYWETSEDNIEKLRQLYSEVEDKIEGIDR

SEQ ID NO: 7 - DNA, 6252 bp, Hordeum vulgare
  barley magnesium-protoporphyrin IX chelatase (Mg-chelatase) H subunit (CHLH), abscisic acid receptor (ABAR), XANTHA-F
taacacgagagccgccaaaacgggaacccctggccaccatgcagctgcagggagggcaacaatcccattcccacgggatcgcaacctttgtccccacgtctcacggacgcgtcgtccagagcctcggaccatctctcgcccgtcagccacagcccccaaagccaaagccaaagcccagcatgagcaagccaccaaacgtaccccttccacgagccataaagcaaaaaaggccccacaaacatcacacgcacggacgggcaccacggagccgtagcatcgagcgaaatgcacggcccaagacggaaaaacaaaaggaaggagaaacggaatccgccaacgcgacgccccacgggaaggcaggagtttgggggggggtgccctttgcggctggggtggatgccacgcgcaggccgcgaacagcgaccgcgggctatccgcccgcctcatcgccatcccccggcggggcgcgcccagggaatccgccgcggccaatcgcagcgcgcgcagccccgatcgcgatccgcttttctcacccccgtgcccccaccgccccgtcacgcgccaacgtgtgccgcctcccgccaccggatgccccggaccgccccgcagccgcttgccagccacctccatagtacttggcaacaaacctatacattcccgagctggcacactctccagctcgctcctcgcctcctcctcctcctcctcccctgcttctgcttcagagggacacgacaacaacacgagcctagagcagagcaagagccgagagccaccctcagcgaagaggagaggtgtgcttggattgccgctccattgctgaataatatatactctgctagtggtagttttttattgattttagccgggaggggataaaagatgtcgtcgctggtgtccgcgccgttcgccacggccaccggggcgcagaagaaggcgcgcgggccgcggcccgcgccgctgcactccttcctgctcaccggccggcgggggcggcgcgcgacgatacggtgcgccgtggccgggaacgggctgttcacgcagaccaacccggacgtgcgccgcgtggttcccgccgagcgcggcctgccgcgggtcaaggtggtctacgtcgtgctggaggcgcagtaccagtcctccgtcaccgccgccgtgatgcagctcaacgccgacccgcgccgcgccgccgagttcgaggtggtgggctacctggtggaggagctgcgggacgcggacacgtacgccgccttctgcgacgacgtggcggccgccaacgtgttcatcggctcgctcatcttcgtggaggagctggcgctcaaggtgcgggacgccgtggcgaagcaccgggaccggatggacgccgtgctcgtcttcccgtccatgccggaggtgatgcggctcaacaagctcggctccttcagcatggcgcagctgggacagtccaagagccccttcttccagctcttcaagcgcaacaagaaggactcgagcggcttcgccgacagcatgctcaagctcgtgcgcacgctgcccaaggtgctcaagtacctgccctccgacaaggcgcaggacgccaggctctacatcctcagcctacagttctggctcggcggctcgccggacaacctccagaacttcctcaagatgatcgccgtctcctacgtgccggccctcaagggcgccgacatcaggtacaacgaccccgtgctcttcctcgacaccggcatctggcacccgctcgcgcctaccatgtacgacgatgtcaaggagtacctcaactggtacggcacccgccgcgacgccaacgacaggctcaagaacccagaggcgcccgtcatcggcctcgtcctgcagcgcagccacattgtcaccggggacgacggacactacgtcgccgtcatcatggagctcgaggccagaggcgccaaggtcatccccatctttgccggcgggctcgacttctccggccccattgagcgctacctggtcgaccccatcaccaagaagccgtttgtgaacgccgtggtgtcgctcaccgggttcgcgctcgtcggcgggccggccaggcaggaccaccccaaggccatcgcctcgctgatgaagctcgacgtgccgtacatcgtcgcgctgccgctcgtgttccagaccacggaggagtggctcaacagcaccttgggccttcaccccatccaggtggcgctgcaggtcgcgctcccggagctcgacggtggcatggagcccatcgtgttcgccggccgggacccgagatcaggcaagccactcctccgtcccccttggcctcttggtctgatttgtctcttgcatcaatgtactttgagctgaactggaaatgctgtttattgtgtttgatgcagggaagtcgcatgcactgcacaagagggtggagcagctctgcaccagagcaatcagatgggcagaactcaagaggaaaactaaggtatgcatctcgcttccacatggatgctatatattggtagtatcatctcttcttgaggtataaatagacaaactcatcgacatattaggctttattagttcaactattagtccctgtcatgctgaaaaccgggactgctaattcaatttgtgcatcaaaccttccaccatgtttaccccaaaaaaaaaaaatagtccacttacaaatagacaaactaatcaacatgttagctcaaataacctcactgtcatgctgaaaactggaactactggttcaatcgatcgattgacttaactgtatctttgcgcccttcacccactgaacagttgttgcttttttacataccaacaccccaagcctgttaatgctgaaagtctaatgcaccctgataatgcctatctatcatcacgtcgaagttcaatgaaaagtagcagcaactcaaaagtttaagctaacaagtgctaactaactgttcttgtacttgctccttcaaatgaaacagaagatcatgtgaggacaaaattaaatacttcactgcagaatctcaaacttcagttatgcgatcatgtcaaattagtggctattaatattttgaggaagtcgaattcctcatgggtgaaaatactctacatgtttctcggttcttgtatgcacaggtttgcaaaagcgaagccataaagtaattacctatttacttgtttgttgcagatggacaaaaaactagccatcaccgtgttcagcttcccaccagacaagggcaatgtcggcactgcagcatacctgaatgtcttcagttccatctattctgtcctccgggatctcaagaaggatggctacaatgtcgagggtcttccagagacacctgaagaactcattgaggaggttattcatgataaggaggcccagttcaacagccccaacctcaatgttgtttaccgcatgaatgtgcgggagtaccaagcactcaccccctacgccaacatgctggaggagaactggggcaagccacctgggcatctcaactctgatggtgagaacctccttgtctatgggaagcagtatggcaacatcttcatcggagtgcagcccacttttggctatgaaggtgatccaatgcggcttctcttctcaaagtctgccagtcctcaccacgggtttgcagcatactatacttttgttgagaagatcttcaaggcagatgccgttctgcacttcggcacgcacgggtcccttgaattcatgcccgggaaacaagttgggatgagtgatgcatgcttcccagatagtctgattggtaacatccccaacatctactactatgcagcaaacaacccttcagaagcaacagttgccaagcggcgaagctatgcaaacaccataagctacctgacaccaccagctgagaatgccggtctgtacaaggggctgaagcagctgtcggagctcatcgcctcttaccagtctctcaaggacacgggacgtggcaaccagattgtgagctcaatcatcagcactgcaaaacagtgtaacctggacaaggatgtcgccttgcccgatgaaggcgaggagctcccagccaatgagcgtgacctcgtcgtcgggaaggtgtatggaaagctcatggagatagagtcacggctactgccatgtggtctgcatgtgataggtgagccaccaactgccgtcgaagctgtggccacattggtgaacatagctgcccttgaccgcccagaggagaacatattctcgctgcccggcattcttgctgcgacagtgggcaggaccattgaagatgtctacaggggcagcgacaagggcatactggctgatgttgaactcctgaagcagatcactgaagcttcacggggtgccgtaggtgcctttgttgagaagagtacaaacagcaaagggcaagttgttgatgttacaagcaaactcagttccatccttggctttggtctctcagagccatgggtggagtacttgtcccagaccaagttcatcagggcggacagagataagctgaggaccttgtttggattcttgggagagtgcctgaagctgattgtggcagacaatgagttgggagccttgaagactgcccttgagggaagctatgttgagcctggccctggtggtgatcccatccgtaacccgaaggttctcccaacagggaagaacatccatgctctcgacccgcagtctatcccgactgcagctgccatgaagagtgccaagattgttgtggaacgtctgctggagcggcaaaaggctgacaatggtggcaagtatcctgagacaattgcacttgtcttgtggggcaccgacaacatcaagacctacggcgagtcactggcccaggtgatgtggatgctcggtgtggagccggttactgatggccttggccgtgtcaaccgtgtggagcctgtcagcattgaggagcttggacgccctaggatcgatgtcgtcgtcaactgctcgggtgtgttcagagaccttttcatcaaccaggtatgaactaataccgcctctgcctttgatttgtatgttttgcagataactaaattattcatcacttaactcatgcactaacgttacacatctgaccaatgcagatgaatctgctggaccgggcagtaaagatggttgctgaactggaagagccaatcgagatgaactatgtgcgcaagcatgccatggagcaggcagaggagctcggtgtctcggtaagagaggcagcaacaaggatcttctcaaatgcatcaggctcctattcgtcgaatgtgaacttggcagtggagaatgcatcatggacagatgagaagcagctccaggacatgtacctgagccgcaagtcttttgcattcgacagtgatgctccaggggtaggcatgctagagaaacgcaagacgtttgagcttgctctagcaacagcagatgccacattccagaacctggactcctcggagatctcattgacagatgtcagccactacttcgactcagacccgacgaagctggtgcaagggctgcggaaggatgggcgggcgccttcctcgtacatagcagacacaaccacagcaaatgcacaggtgcgtacattgtcggagacggtgcgtcttgatgcaaggacaaagctactgaaccctaggtggtatgaggggatgatgaagagtggctatgagggagttagagagatcgagaagcggctgacaaatactgttggatggagtgcaacatccgggcaggtggacaactgggtttacgaggaagcaaataccacattcatcgaagatgaagagatgaggaagaggctgatggacactaatcccaattcgttcaggaagctgcttcaaaccttcctagaagcaaatggcagaggctactgggagacatcagaggataacttggaaaggctcagggagctctactcggaggttgaagacaagatcgaaggaattgaccggtgaactcatttcactgatcaaccagttccagcacttgaccaagaaggaattcttctgcctcgttgagttgaaacttgtataatttgtaatttatataagttgtaacatgcaatacagacctttaacaaagagggtggttcgttgtgaatgtaaaaaggaagcatgagtagaagttcttttttggggaatattggtactttgctgtcacatgttcattgacagatatagtaacagagagtggggtatcatttttatctgttttaacaacagaacatttgaagttagcattcttcgttgttatctttacattgaactgcaacggcccaa

SEQ ID NO: 8 - protein, 1381 aa, Hordeum vulgare
  barley magnesium-protoporphyrin IX chelatase (Mg-chelatase) H subunit (CHLH), abscisic acid receptor (ABAR), XANTHA-F
MSSLVSAPFATATGAQKKARGPRPAPLHSFLLTGRRGRRATIRCAVAGNGLFTQTNPDVRRVVPAERGLPRVKVVYVVLEAQYQSSVTAAVMQLNADPRRAAEFEVVGYLVEELRDADTYAAFCDDVAAANVFIGSLIFVEELALKVRDAVAKHRDRMDAVLVFPSMPEVMRLNKLGSFSMAQLGQSKSPFFQLFKRNKKDSSGFADSMLKLVRTLPKVLKYLPSDKAQDARLYILSLQFWLGGSPDNLQNFLKMIAVSYVPALKGADIRYNDPVLFLDTGIWHPLAPTMYDDVKEYLNWYGTRRDANDRLKNPEAPVIGLVLQRSHIVTGDDGHYVAVIMELEARGAKVIPIFAGGLDFSGPIERYLVDPITKKPFVNAVVSLTGFALVGGPARQDHPKAIASLMKLDVPYIVALPLVFQTTEEWLNSTLGLHPIQVALQVALPELDGGMEPIVFAGRDPRSGKPLLRKSHALHKRVEQLCTRAIRWAELKRKTKMDKKLAITVFSFPPDKGNVGTAAYLNVFSSIYSVLRDLKKDGYNVEGLPETPEELIEEVIHDKEAQFNSPNLNVVYRMNVREYQALTPYANMLEENWGKPPGHLNSDGENLLVYGKQYGNIFIGVQPTFGYEGDPMRLLFSKSASPHHGFAAYYTFVEKIFKADAVLHFGTHGSLEFMPGKQVGMSDACFPDSLIGNIPNIYYYAANNPSEATVAKRRSYANTISYLTPPAENAGLYKGLKQLSELIASYQSLKDTGRGNQIVSSIISTAKQCNLDKDVALPDEGEELPANERDLVVGKVYGKLMEIESRLLPCGLHVIGEPPTAVEAVATLVNIAALDRPEENIFSLPGILAATVGRTIEDVYRGSDKGILADVELLKQITEASRGAVGAFVEKSTNSKGQVVDVTSKLSSILGFGLSEPWVEYLSQTKFIRADRDKLRTLFGFLGECLKLIVADNELGALKTALEGSYVEPGPGGDPIRNPKVLPTGKNIHALDPQSIPTAAAMKSAKIVVERLLERQKADNGGKYPETIALVLWGTDNIKTYGESLAQVMWMLGVEPVTDGLGRVNRVEPVSIEELGRPRIDVVVNCSGVFRDLFINQMNLLDRAVKMVAELEEPIEMNYVRKHAMEQAEELGVSVREAATRIFSNASGSYSSNVNLAVENASWTDEKQLQDMYLSRKSFAFDSDAPGVGMLEKRKTFELALATADATFQNLDSSEISLTDVSHYFDSDPTKLVQGLRKDGRAPSSYIADTTTANAQVRTLSETVRLDARTKLLNPRWYEGMMKSGYEGVREIEKRLTNTVGWSATSGQVDNWVYEEANTTFIEDEEMRKRLMDTNPNSFRKLLQTFLEANGRGYWETSEDNLERLRELYSEVEDKIEGIDR

SEQ ID NO: 9 - DNA, 5071 bp, Antirrhinum majus
  snapdragon magnesium-protoporphyrin IX chelatase(Mg-chelatase) H subunit (CHLH), abscisic acid receptor (ABAR), olive gene
agtacatttttgtttttactcatttatttttcctctttctggtaaaaggccaagtacaatcctcttctcagaaaaaaatatcagtttctttcagctaagggccacaaactcacaaactaaaagaatctttccattttccaatcaaaaaggtactcataataatgacatcaaaaaccaactataaaaaaaaaaaaaacaaaattgacatgaaaatctctgatcaattaatctatccacactccttccctcctctccaattccccgaaaatccaaccaaccaatcagaatccaagaaacttcatcacaaatgaccccatcagataaccagactacaaacaattaaacacactcccatgaaactgaactcagcctcagaatactgtctaccatctatgccacgtgtccctttctctcaccactaacaagaaaaactaacatttttcacctctgttttcatattatataaaactcaccactctgctaaaagaaaagaagaacaacagcttctacaaaaaaagctgaagaaaatcttgaacttgaacagagtctctcaggttcatagcttcacaacccatttctctaatctttattctatttgattctcttgtgtacatattgtgtgtgtgtcatcagggttatttttcttttttttgttgttgttgtactctgtttcttgaaaataaacagagtttgtgcaaaaatggcttcattggtatcttcgccatttacattgccaaactctaaagttgaaaacttgtcatcaatttcacaaaagcattactttctccactcatttctccccaagaaactcaaccaaaacaacaagtcacaaaaattcaaatgtgttgcaattggcaatggtttgttcactcagaccacacaggaggttcgtcgaatcgtgcccgaaaacttaaaaggccttccgactgttaaaatcgtgtacgtcgtgttagaggctcagtaccaatcatcactcacagctgcagtgcaaagccttaaccagaatgggaaatatgcttcttttgaggtggtggggtacttagttgaggagcttagagaccccaacacatacaagtcgttatgtaaagatctcgaggacgcgaatatctttatcgggtcgttgatctttgttgaggagttggccttgaaggttaaggatgcagtggagaaagaaagggagaggcttgatgctgtgttggtattcccatcaatgccagaagtaatgaggctcaacaaattggggtcgttcagtatgtcgcagctcgggcagtcgaagagcccgtttttccagctgttcaagaagaacaagtcatctgctggctttgctgacagtatgttgaagcttgtgaggacactacctaaggttttgaagtacttgccgagcgataaggcgcaggatgctagattgtacatacttagcttgcagttttggcttggaggttcacctgataatctggtgaatttcttgaagatgatttctggttcttatattcctgctctaaaagggacaaagatcgagtattcggacccggtattgtatttagataccgggatatggcaccctttggcgccttgtatgtatgatgatgttaaggagtatttgaattggtatggtactagaagggatgccaatgagaagctgaagagttcgaaagcacctattgttggactggttttacagaggagtcatatagttactggtgatgagagtcactatgtggctgtgatcatggagctggaagctagaggggctaaagtgataccaattttcgccggtggtctcgacttttcaggaccagttgaaaagtattttattgatccaatcacgaagaagcctatggtgaattctgtgatttcgcttacgggatttgctcttgttggaggtccagcaaggcaggaccatccaagggctattgaggccctgatgaagcttgatgtgccttacattgtggctgtgccgttggtgtttcaaacaacggaagaatggttgaacagcacattgggacttcaccctgttcaggtggctctacaagttgctctgccagagcttgatggaggcatggaaccaatcattttcgcgggacgtgatcctagaacaggaaaatcacatgctcttcacaaaagagtggagcagctatgcacaagagctatcaactggggtaatctgacgagaaagaagaagactgaaaagagggtagctatcactgtatttagcttccccccagacaaaggcaatgtcggaactgctgcttacctgaatgtttttgcctccattttctcggtactcaaagatttaaagaaggatggctacaacgtcgagggccttccagaaactgctgaagcgttaatagaagagataattcatgacaaagaggctcaatttaacagtccaaatcttaacatagcctacaaaatgaacgtgcgggagtaccaggctctgactccctattctgctgctcttgaagaaaactggggaaaaccccctggtaatctgaacgccgacggagaaaatctcctggtgtacggtaaacagtacggaaatgtctttattggcgtccagccaacgtttggttatgagggagatccaatgaggcttctgttctctaaatctgcgagtccacaccatggatttgctgcttactactcttttgtagagaaaatctttaaagctgatgctgtccttcactttggcactcatggttcattagaattcatgcctgggaaacaagttggcatgagtgatgcttgtttccctgacagtcttattggaaacattccaaacatgtactattatgcagctaacaatccatctgaagccaccattgccaagcgtcgtagttatgcaaataccattagttacttgacgcctccagctgaaaatgctgggctatacaagggacttaagcagcttggtgaacttatctcctcctatcaatctctaaaagactctggccgtggtcctcaaattgtgagctctatcatcagcactgccaggcaatgcaatcttgacaaggatgtggaacttcctgaagagggagctgagatttctgccaaagaacgagatctcgtggttggaaaagtttattccaagattatggagattgaatcccggttactgccatgtgggcttcatatcattggcgaacctccaactgctatggaagcagttgccacactggttaatattgctgcattggatcgtccagaagaagggatttccgctcttacatcaatattggctgagactgtgggcagaagtatcgaggatgtttaccgaggaagtgacaagggtattttgaaagacgtggaacttcttcgtcagataactgaagcgtcacgtggtgcaattactgcatttgtggagcgaaccactaacgacaaaggccaggtggttgatgtttccaacaagttgacatcaatacttggatttggtattaacgaaccgtgggttcaatacctgtcgaataccaagttttacagagctgacagagagaagctcagagtcctgttccagttcttaggggaatgcttgaagctagtagtggctaataatgaggtgggaagtttgaaacaagctttggaaggtaagttcgtggagccgggtccaggaggggatccgattaggaatcctaaagtgttgcccactggaaagaatatccatgctttggacccacagtctattcccacaactgcagctatgcaaagtgcgatggtggtcgttgataggttgctcgagaggcaaaaggccgacaatggagggaaatttcccgagactgttgctcttgtactctggggaacagataacatcaagacttatggggaatcactggctcaagtcctttggatgattggagttaaacctgtttcagatacttttggacgcgtgaatcgggttgaacctgtaagccttgaagaacttggaaggccaagggttgatgtggttgtcaattgttctggtgtattcagagatcttttcattaatcagatgaatctccttgatcgggcagtgaagatggttgccgagttagatgagcctgtagaacaaaactttgtaagaaaacacgcactagaacaagcaaaagaattaggagttgaagtgcgtgaggctgcttctcgtatcttctctaatgcctcgggttcatactcttccaacataaatctcgccgtcgagaattcatcgtggaatgatgagaagcaacttcaagacatgtacttgagccgaaagtcatttgcattcgactctgatgctccaggtgttggcatgactgagaaaagaaaaatctttgaaatggccctcagcacagctgatgccactttccaaaatctcgactcctcagagatctcgctcacagatgttagtcactactttgattcagatcccacaaaccttgttcagaaccttaggaaagacggaaagaagccgagtgcatacattgctgacacaaccacagctaattcacaggtacgtacattgtctgagacggtccgtcttgacgcaagaaccaagttgctgaatccaaaatggtacgaaggcatgctgtcaagtgggtacgagggagttcgtgagattgagaagcggctgactaacaccgttggctggagtgcaacttcaggacaagtcgataattgggtgtatgaagaggcaaatactactttcatcgaagatgagcaaatgttgaacagactgatgaacacgaaccctaactcttttaggaagttgcttcagacgtttttggaggctaatgggcgtggatactgggaaacctctgccgagaacattgagagattgaggcaattgtactcagaagtcgaagacaagattgaaggaattgatcgttgagtcttgattcaaacaaactgttgttcaactgcaaataacattatatacctccttttcctcgtagtaggatggatctttccataatttggagcaccatttttttccttctcactaattgcccactgtattcattttcccagggtggttgtcttcattgtatgtaacttgtgagacactgattcactataaattcaactttatgccattgctatcaaaaaaaaaaaaaaaaaa

SEQ ID NO: 10 - protein, 1379 aa, Antirrhinum majus
  snapdragon magnesium-protoporphyrin IX chelatase(Mg-chelatase) H subunit (CHLH), abscisic acid receptor (ABAR), olive gene
MASLVSSPFTLPNSKVENLSSISQKHYFLHSFLPKKLNQNNKSQKFKCVAIGNGLFTQTTQEVRRIVPENLKGLPTVKIVYVVLEAQYQSSLTAAVQSLNQNGKYASFEVVGYLVEELRDPNTYKSLCKDLEDANIFIGSLIFVEELALKVKDAVEKERERLDAVLVFPSMPEVMRLNKLGSFSMSQLGQSKSPFFQLFKKNKSSAGFADSMLKLVRTLPKVLKYLPSDKAQDARLYILSLQFWLGGSPDNLVNFLKMISGSYIPALKGTKIEYSDPVLYLDTGIWHPLAPCMYDDVKEYLNWYGTRRDANEKLKSSKAPIVGLVLQRSHIVTGDESHYVAVIMELEARGAKVIPIFAGGLDFSGPVEKYFIDPITKKPMVNSVISLTGFALVGGPARQDHPRAIEALMKLDVPYIVAVPLVFQTTEEWLNSTLGLHPVQVALQVALPELDGGMEPIIFAGRDPRTGKSHALHKRVEQLCTRAINWGNLTRKKKTEKRVAITVFSFPPDKGNVGTAAYLNVFASIFSVLKDLKKDGYNVEGLPETAEALIEEIIHDKEAQFNSPNLNIAYKMNVREYQALTPYSAALEENWGKPPGNLNADGENLLVYGKQYGNVFIGVQPTFGYEGDPMRLLFSKSASPHHGFAAYYSFVEKIFKADAVLHFGTHGSLEFMPGKQVGMSDACFPDSLIGNIPNMYYYAANNPSEATIAKRRSYANTISYLTPPAENAGLYKGLKQLGELISSYQSLKDSGRGPQIVSSIISTARQCNLDKDVELPEEGAEISAKERDLVVGKVYSKIMEIESRLLPCGLHIIGEPPTAMEAVATLVNIAALDRPEEGISALTSILAETVGRSIEDVYRGSDKGILKDVELLRQITEASRGAITAFVERTTNDKGQVVDVSNKLTSILGFGINEPWVQYLSNTKFYRADREKLRVLFQFLGECLKLVVANNEVGSLKQALEGKFVEPGPGGDPIRNPKVLPTGKNIHALDPQSIPTTAAMQSAMVVVDRLLERQKADNGGKFPETVALVLWGTDNIKTYGESLAQVLWMIGVKPVSDTFGRVNRVEPVSLEELGRPRVDVVVNCSGVFRDLFINQMNLLDRAVKMVAELDEPVEQNFVRKHALEQAKELGVEVREAASRIFSNASGSYSSNINLAVENSSWNDEKQLQDMYLSRKSFAFDSDAPGVGMTEKRKIFEMALSTADATFQNLDSSEISLTDVSHYFDSDPTNLVQNLRKDGKKPSAYIADTTTANSQVRTLSETVRLDARTKLLNPKWYEGMLSSGYEGVREIEKRLTNTVGWSATSGQVDNWVYEEANTTFIEDEQMLNRLMNTNPNSFRKLLQTFLEANGRGYWETSAENIERLRQLYSEVEDKIEGIDR

SEQ ID NO: 11 - DNA, 4578 bp, Nicotiana tabacum
  common tobacco magnesium-protoporphyrin IX chelatase (Mg-chelatase) H subunit (CHLH), abscisic acid receptor (ABAR)
agccactactctccacataaaactataaacttagaacatttcacttgaaaaaagagaggaaaaaagtgaagcagaaatcttttctcaaaacacaatctataggaagttaaattcaacttccacacttccaagattcttgtttcaagtttcgtttagtttttttttcttggttttttttatagttttctgtacaattttgtgtagaatcaagaaacgaaagagttaaagtttgaaacttttttacaagtttgaaacaatggcttctttggtttcttcaccatttacattgccaaattcaaaagtagaacacttgtcatccatttctcaaaagcattactttcttcactcatttcttcccaagaaaataaaccccacttactcaaaatcaccaaagaaattccaatgtaatgctattggcaatggtttattcactcaaacaactcaagaagttaggagaattgtgcctgaaaatactcagggacttgctactgtgaaaatagtctatgttgtattggaagctcagtaccaatcatcacttactgctgctgttcagacactgaacaagaatggtcagtttgcttcttttgaggttgtggggtacttggttgaggagcttagagatgagaatacttataaaatgttttgtaaagatcttgaggatgcaaatgtgtttattggttcattgatttttgtggaagaattggctttaaaggtaaaatctgcagtggagaaagaaagggacagacttgatgcagttttggtgtttccatcaatgcctgaggtgatgaggttgaacaagttgggatcttttagtatgtcacaattggggcaatcaaagagtccattttttgagcttttcaagaagaagaaaccttcttctgcaggtttttctgatcagatgttgaagcttgtgagaacattgcctaaggttttgaagtatttaccaagtgataaagctcaagatgctaggttgtacatactaagtttgcagttttggctaggaggttcacctgataatttggtgaatttcttgaaaatgatttctggttcttatgttcctgctcttaaagggatgaaaatcgactactcggatccggttttgtacttggataatggaatttggcaccctttggctccttgtatgtatgatgatgtgaaggagtatttgaattggtatgcaacaaggagagatactaatgagaaactcaagagttcaaatgctcctgttgttgggctggttttgcaaaggagtcatattgttacttgtgatgagagtcactatgtggctgtgatcatggaattggaggcaaagggggctaaagttatcccaatttttgccggtgggctagacttttcgaggccaattgagagatatttcattgatcctattacaaagaagccttttgtgaattcagtaatatcactttctggttttgcacttgttggagggccagcaagacaagaccatccaagggcaatagaggctttgatgaaacttgatgtgccttatattgtggcattgcctttggttttccaaacaacagaggaatggttgaacagtactttggggctgcaccctattcaggtggctctacaagttgctctccctgagctggatggaggaatggagcccatcgtattcgccggtcgcgatccaagaacagggaaatcacatgctcttcacaaaagagtggagcagctttgcaccagggcaatcaaatggggagagttaaagagaaaaacaaaggctgagaagaggttggcaatcactgtcttcagctttcctccagacaaaggcaatgtcggaactgctgcatacttgaatgtctttgcctccatatactctgttctcaaagatctcaagaaagacggctacaacgttgaggggctgcctgagacttctgcacaacttattgaagaagtaattcacgacaaagaagctcagttcagcagcccaaatcttaacatagcttacaagatgaatgttagagaataccagaagctaaccccctatgctactgctcttgaagaaaactgggggaaagcacctggtaatttgaactctgatggagaaaacctcttggtatatggtaaacagtacggcaatgtctttatcggtgttcagcccacgtttggatacgagggtgacccgatgagacttctgttctccaaatcagctagccctcaccatggttttgctgcatactattcctttgtggagaaaattttcaaagctgatgcagttctccactttggtactcatggttctcttgagttcatgccaggtaaacaggtgggaatgagcgatgcttctttccctgatagtctcattggaaacattcccaatgtctattactatgcagcaaacaacccatctgaagcaactattgccaaacgaaggagttatgcgaataccattagctacttgactcctccggctgagaatgctggactctacaagggactcaagcagctcagtgagctcatttcctcataccaatctctgaaagactcaggccgtggccaacagattgtgaactctatcatcagtacagctagacagtgtaatcttgacaaggatgttgatcttccagaagaaggggaggaaatctcggccaaagagcgtgaccttgtggtaggaaaagtatactctaagattatggagatcgagtctcgtcttcttccgtgtggacttcacatcattggtgaacctccaaccgcgatggaagcagttgctactcttgtcaatattgcgacattggaccgtcctgaagagggtatttctgcccttccatctatattggctgcgacggttggaagaagcattgaggagatttacagaggcaatgaccagggcatcttacgagatgtggagctgctccgtcaaattactgaggcatcacgtggagcaatatcagcatttgttgaacgtacgacaaacaacaagggtcaggttgtgaatgtcaatgacaagctaacctcaatccttggttttggtataaatgaaccatggatccagtatttgtcaaacacccaattttacagagctgatagggacaagctcagagttctattccagttcttgggagagtgtctgaagctaattgtcgctaacaacgaggtgggaagcttgaaacaggctctagaagggaaatatgttgaaccaggtccaggaggggatccgatcagaaacccgaaagttttgcctactgggaaaaacatccatgctttggacccacaagctattcccacaatagcagcagtgcagagtgccaaaattgttgttgaaagattgttggagaggcaaaaggccgacaacgggggcaagtacccggagactgttgctctggttctttggggaacagacaacatcaagacctatggagagtcattggcacaggttatgtggatgattggtgttaggccagttacagactcgttaggacgggttaaccgggtggaacctgttagccttgaagagcttggaaggcctagagttgatgttgttgtcaactgctctggggtgttcagagatctcttcatcaatcagatgaatctccttgaccgagcagtcaagatggttgcagagctcgacgagccagaagaccaaaactacgtcaggaaacatgcactagaacaagcaaaaacactcggagttgatgttcgtgaagctgctacaaggatcttctcaaatgcttcaggatcttactcctccaacattaaccttgctgttgagaattcaacatggaatgatgagaagcaacttcaagacatgtacttgagccgaaagtcatttgcatttgactgtgatgcccctggtgttggcatgactgagaagaggaaagtttttgagatggctcttagcacggctgatgccacattccagaaccttgactcatctgaaatttcattcacagacgtgagtcactacttcgattcagacccaaccaaccttgtgcaaaacctcaggaaagacgggaagaagcctagtgcatacattgctgacaccactactgctaatgctcaggtacgtacgttgtctgagactgtgaggcttgacgcaaggacaaagttgttgaaccccaagtggtatgaaggcatgctatccactggctacgagggtgttcgtgagattgagaaacgattaactaacacagtggggtggagtgcaacttcaggccaagttgataattgggtggatgaagaagccaacacaaccttcattcaagatcaggagatgttgaacaggctcatgaacacaaatccaaattctttcaggaagttgcttcagacattcttggaagccaacgggcgtggatactgggaaacttctgcagagaacattgagaaactcaagcaattatactcagaagttgaagacaagattgagggaatcgatcgataaatgtatagcaaaaagaatgatctctgattattgcctgtttgttcctaactgtttctgatgtgaattcctttgacagtccccagtgtaattttgttcatttttggggatgtcctacttctatgagaaaatactgcttccatatattcaaatttgagcttgaaaaaaaaaaaaaa

SEQ ID NO: 12 - protein, 1382 aa, Nicotiana tabacum
  common tobacco magnesium-protoporphyrin IX chelatase (Mg-chelatase) H subunit (CHLH), abscisic acid receptor (ABAR)
MASLVSSPFTLPNSKVEHLSSISQKHYFLHSFLPKKINPTYSKSPKKFQCNAIGNGLFTQTTQEVRRIVPENTQGLATVKIVYVVLEAQYQSSLTAAVQTLNKNGQFASFEVVGYLVEELRDENTYKMFCKDLEDANVFIGSLIFVEELALKVKSAVEKERDRLDAVLVFPSMPEVMRLNKLGSFSMSQLGQSKSPFFELFKKKKPSSAGFSDQMLKLVRTLPKVLKYLPSDKAQDARLYILSLQFWLGGSPDNLVNFLKMISGSYVPALKGMKIDYSDPVLYLDNGIWHPLAPCMYDDVKEYLNWYATRRDTNEKLKSSNAPVVGLVLQRSHIVTCDESHYVAVIMELEAKGAKVIPIFAGGLDFSRPIERYFIDPITKKPFVNSVISLSGFALVGGPARQDHPRAIEALMKLDVPYIVALPLVFQTTEEWLNSTLGLHPIQVALQVALPELDGGMEPIVFAGRDPRTGKSHALHKRVEQLCTRAIKWGELKRKTKAEKRLAITVFSFPPDKGNVGTAAYLNVFASIYSVLKDLKKDGYNVEGLPETSAQLIEEVIHDKEAQFSSPNLNIAYKMNVREYQKLTPYATALEENWGKAPGNLNSDGENLLVYGKQYGNVFIGVQPTFGYEGDPMRLLFSKSASPHHGFAAYYSFVEKIFKADAVLHFGTHGSLEFMPGKQVGMSDASFPDSLIGNIPNVYYYAANNPSEATIAKRRSYANTISYLTPPAENAGLYKGLKQLSELISSYQSLKDSGRGQQIVNSIISTARQCNLDKDVDLPEEGEEISAKERDLVVGKVYSKIMEIESRLLPCGLHIIGEPPTAMEAVATLVNIATLDRPEEGISALPSILAATVGRSIEEIYRGNDQGILRDVELLRQITEASRGAISAFVERTTNNKGQVVNVNDKLTSILGFGINEPWIQYLSNTQFYRADRDKLRVLFQFLGECLKLIVANNEVGSLKQALEGKYVEPGPGGDPIRNPKVLPTGKNIHALDPQAIPTIAAVQSAKIVVERLLERQKADNGGKYPETVALVLWGTDNIKTYGESLAQVMWMIGVRPVTDSLGRVNRVEPVSLEELGRPRVDVVVNCSGVFRDLFINQMNLLDRAVKMVAELDEPEDQNYVRKHALEQAKTLGVDVREAATRIFSNASGSYSSNINLAVENSTWNDEKQLQDMYLSRKSFAFDCDAPGVGMTEKRKVFEMALSTADATFQNLDSSEISFTDVSHYFDSDPTNLVQNLRKDGKKPSAYIADTTTANAQVRTLSETVRLDARTKLLNPKWYEGMLSTGYEGVREIEKRLTNTVGWSATSGQVDNWVDEEANTTFIQDQEMLNRLMNTNPNSFRKLLQTFLEANGRGYWETSAENIEKLKQLYSEVEDKIEGIDR

SEQ ID NO: 13 - DNA, 36 bp, Artificial Sequence
  Description of Artificial SequencePCR forward primer for isolation of ABAR/CHLH gene open reading frame (ORF)
taggcgcgccaaaatggcttcgcttgtgtattctcc

SEQ ID NO: 14 - DNA, 35 bp, Artificial Sequence
  Description of Artificial SequencePCR reverse primer for isolation of ABAR/CHLH gene open reading frame (ORF)
ggactagtttatcgatcgatcccttcgatcttgtc

SEQ ID NO: 15 - DNA, 34 bp, Artificial Sequence
  Description of Artificial Sequence amplificationforward primer for Gene-specific DNA fragment covering parts of 5′-untranslated region and coding region of full length ABAR cDNA
acgggtaccgagagaatcataaactcccacttgg

SEQ ID NO: 16 - DNA, 32 bp, Artificial Sequence
  Description of Artificial Sequence amplificationreverse primer for Gene-specific DNA fragment covering parts of 5′-untranslated region and coding region of full length ABAR cDNA
tcgtctagagagtgagtcattggtgtcccttc

SEQ ID NO: 17 - DNA, 29 bp, Artificial Sequence
  Description of Artificial Sequence amplificationforward primer for ABAR/CHLH gene-specific 653-bp fragment
cggtctagaacagagattctgtggttggg

SEQ ID NO: 18 - DNA, 31 bp, Artificial Sequence
  Description of Artificial Sequence amplificationreverse primer for ABAR/CHLH gene-specific 653-bp fragment
atacccgggggcacttgccattgctgctgtt

SEQ ID NO: 19 - DNA, 31 bp, Artificial Sequence
  Description of Artificial Sequence amplificationforward primer for ABAR/CHLH gene-specific 596-bp fragment
atagagctcttttgccgtgcgggcttcacgt

SEQ ID NO: 20 - DNA, 31 bp, Artificial Sequence
  Description of Artificial Sequence amplificationreverse primer for ABAR/CHLH gene-specific 596-bp fragment
ctgtacgtaggcacttgccattgctgctgtt

SEQ ID NO: 21 - DNA, 31 bp, Artificial Sequence
  Description of Artificial SequencePCR amplification forward primer for ABAR/CHLH gene-specific 351-bp fragment sense orientation insert
ccgctcgaggttcttggatactggaatttgg

SEQ ID NO: 22 - DNA, 32 bp, Artificial Sequence
  Description of Artificial SequencePCR amplification reverse primer for ABAR/CHLH gene-specific 351-bp fragment sense orientation insert
acgcgtcgacggtccaccaacaagagcaaaac

SEQ ID NO: 23 - DNA, 30 bp, Artificial Sequence
  Description of Artificial SequencePCR amplification forward primer for ABAR/CHLH gene-specific 351-bp fragment antisense orientation insert
cggaattcggtccaccaacaagagcaaaac

SEQ ID NO: 24 - DNA, 29 bp, Artificial Sequence
  Description of Artificial SequencePCR amplification reverse primer for ABAR/CHLH gene-specific 351-bp fragment antisense orientation insert
gactagtgttcttggatactggaatttgg

SEQ ID NO: 25 - DNA, 653 bp, Artificial Sequence
  Description of Artificial Sequencestable expression interfering RNA (RNAi) construct sense arm
acagagattctgtggttgggaaagtttattccaagattatggagattgaatcaaggcttttgccgtgcgggcttcacgtcattggagagcctccatccgccatggaagctgtggccacactggtcaacattgctgctctagatcgtccggaggatgagatttcagctcttccttctatattagctgagtgtgttggaagggagatagaggatgtttacagaggaagcgacaagggtatcttgagcgatgtagagcttctcaaagagatcactgatgcctcacgtggcgctgtttccgcctttgtggaaaaaacaacaaatagcaaaggacaggtggtggatgtgtctgacaagcttacctcgcttcttgggtttggaatcaatgagccatgggttgagtatttgtccaacaccaagttctacagggcgaacagagataagctcagaacagtgtttggtttccttggagagtgcctgaagttggtggtcatggacaacgaactagggagtctaatgcaagctttggaaggcaagtacgtcgagcctggccccggaggtgatcccatcagaaacccaaaggtcttaccaaccggtaaaaacatccatgccttagatcctcaggctattcccacaacagcagcaatggcaagtgcc

SEQ ID NO: 26 - DNA, 596 bp, Artificial Sequence
  Description of Artificial Sequencestable expression interfering RNA (RNAi) construct antisense arm
ttttgccgtgcgggcttcacgtcattggagagcctccatccgccatggaagctgtggccacactggtcaacattgctgctctagatcgtccggaggatgagatttcagctcttccttctatattagctgagtgtgttggaagggagatagaggatgtttacagaggaagcgacaagggtatcttgagcgatgtagagcttctcaaagagatcactgatgcctcacgtggcgctgtttccgcctttgtggaaaaaacaacaaatagcaaaggacaggtggtggatgtgtctgacaagcttacctcgcttcttgggtttggaatcaatgagccatgggttgagtatttgtccaacaccaagttctacagggcgaacagagataagctcagaacagtgtttggtttccttggagagtgcctgaagttggtggtcatggacaacgaactagggagtctaatgcaagctttggaaggcaagtacgtcgagcctggccccggaggtgatcccatcagaaacccaaaggtcttaccaaccggtaaaaacatccatgccttagatcctcaggctattcccacaacagcagcaatggcaagtgcc

SEQ ID NO: 27 - DNA, 407 bp, Artificial Sequence
  Description of Artificial Sequencestable expression interfering RNA (RNAi) construct loop sequence fragment of GUS gene
cccgggtggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgta

SEQ ID NO: 28 - DNA, 351 bp, Artificial Sequence
  Description of Artificial Sequencechical-regulated inducible interfering RNA (RNAi) construct sense and antisense arm
gttcttggatactggaatttggcatccacttgctccaaccatgtacgatgatgtgaaggagtactggaactggtatgacactagaagggacaccaatgactcactcaagaggaaagatgcaacggttgtcggtttagtcttgcagaggagtcacattgtgactggtgatgatagtcactatgtggctgttatcatggagcttgaggctagaggtgctaaggtcgttcctatattcgcaggagggttggatttctctggtccagtagagaaatatttcgtagacccggtgtcgaaacagcccatcgtaaactctgctgtctccttgactggttttgctcttgttggtggacc

SEQ ID NO: 29 - DNA, 1073 bp, Artificial Sequence
  Description of Artificial Sequenceantisense-ABAR.CHLH construct
gagagaatcataaactcccacttggagctcaaaaagtgtaagagacaaccaacaaaaaacgattcatctcttctcctatcctctcctcttcgaattcaacgtttggagaatccagcagccgcaaaatggcttcgcttgtgtattctccattcactctatccacttctaaagcagagcatctctcttcgctcactaacagtaccaaacattctttcctccggaagaaacacagatcaaccaaaccagccaaatctttcttcaaggtgaaatctgctgtatctggaaacggcctcttcacacagacgaacccggaggtccgtcgtatagttccgatcaagagagacaacgttccgacggtgaaaatcgtctacgtcgtcctcgaggctcagtaccagtcttctctcagtgaagccgtgcaatctctcaacaagacttcgagattcgcatcctacgaagtggttggatacttggtcgaggagcttagagacaagaacacttacaacaacttctgcgaagaccttaaagacgccaacatcttcattggttctctgatcttcgtcgaggaattggcgattaaagttaaggatgcggtggagaaggagagagacaggatggacgcagttcttgtcttcccttcaatgcctgaggtaatgagactgaacaagcttggatcttttagtatgtctcaattgggtcagtcaaagtctccgtttttccaactcttcaagaggaagaaacaaggctctgctggttttgccgatagtatgttgaagcttgttaggactttgcctaaggttttgaagtacttacctagtgacaaggctcaagatgctcgtctctacatcttgagtttacagttttggcttggaggctctcctgataatcttcagaattttgttaagatgatttctggatcttatgttccggctttgaaaggtgtcaaaatcgagtattcggatccggttttgttcttggatactggaatttggcatccacttgctccaaccatgtacgatgatgtgaaggagtactggaactggtatgacactagaagggacaccaatgactcactc

SEQ ID NO: 30 - DNA, 34 bp, Artificial Sequence
  Description of Artificial SequencePCR amplification forward primer for full-length ABAR/CHLH coding sequence
tcgtcgacaaaatggcttcgcttgtgtattctcc

SEQ ID NO: 31 - DNA, 36 bp, Artificial Sequence
  Description of Artificial SequencePCR amplification reverse primer for full-length ABAR/CHLH coding sequence
tagcggccgctatcgatcgatcccttcgatcttgtc

SEQ ID NO: 32 - protein, 6 aa, Artificial Sequence
  Description of Artificial Sequence6xHis tag
HHHHHH

SEQ ID NO: 33 - DNA, 25 bp, Artificial Sequence
  Description of Artificial Sequenceforward primer for RNA gel blotting for ABAR/CHLH expression
ctgagtgtgttggaagggagataga

SEQ ID NO: 34 - DNA, 25 bp, Artificial Sequence
  Description of Artificial Sequencereverse primer for RNA gel blotting for ABAR/CHLH expression
ctctaccaacctctcaaccacaatc

SEQ ID NO: 35 - DNA, 23 bp, Artificial Sequence
  Description of Artificial Sequence real-time PCRRD29A gene-specific forward primer
atcacttggctccactgttgttc

SEQ ID NO: 36 - DNA, 26 bp, Artificial Sequence
  Description of Artificial Sequence real-time PCRRD29A gene-specific reverse primer
acaaaacacacataaacatccaaagt

SEQ ID NO: 37 - DNA, 20 bp, Artificial Sequence
  Description of Artificial Sequence real-time PCRMYB2 gene-specific forward primer
tgctcgttggaaccacatcg

SEQ ID NO: 38 - DNA, 22 bp, Artificial Sequence
  Description of Artificial Sequence real-time PCRMYB2 gene-specific reverse primer
accacctattgccccaaagaga

SEQ ID NO: 39 - DNA, 20 bp, Artificial Sequence
  Description of Artificial Sequence real-time PCRMYC2 gene-specific forward primer
tcatacgacggttgccagaa

SEQ ID NO: 40 - DNA, 24 bp, Artificial Sequence
  Description of Artificial Sequence real-time PCRMYC2 gene-specific reverse primer
agcaacgtttacaagctttgattg

SEQ ID NO: 41 - DNA, 23 bp, Artificial Sequence
  Description of Artificial Sequence real-time PCROST1 gene-specific forward primer
tggagttgcgagattgatgagag

SEQ ID NO: 42 - DNA, 25 bp, Artificial Sequence
  Description of Artificial Sequence real-time PCROST1 gene-specific reverse primer
cctgtggttgattatctcccttttt

SEQ ID NO: 43 - DNA, 24 bp, Artificial Sequence
  Description of Artificial Sequence real-time PCRABI1 gene-specific forward primer
agagtgtgcctttgtatggtttta

SEQ ID NO: 44 - DNA, 25 bp, Artificial Sequence
  Description of Artificial Sequence real-time PCRABI1 gene-specific reverse primer
catcctctctctacaatagttcgct

SEQ ID NO: 45 - DNA, 25 bp, Artificial Sequence
  Description of Artificial Sequence real-time PCRABI2 gene-specific forward primer
gatggaagattctgtctcaacgatt

SEQ ID NO: 46 - DNA, 24 bp, Artificial Sequence
  Description of Artificial Sequence real-time PCRABI2 gene-specific reverse primer
gtttctccttcactatctcctccg

SEQ ID NO: 47 - DNA, 24 bp, Artificial Sequence
  Description of Artificial Sequence real-time PCRABI3 gene-specific forward primer
tccattagacagcagtcaaggttt

SEQ ID NO: 48 - DNA, 24 bp, Artificial Sequence
  Description of Artificial Sequence real-time PCRABI3 gene-specific reverse primer
ggtgtcaaagaactcgttgctatc

SEQ ID NO: 49 - DNA, 22 bp, Artificial Sequence
  Description of Artificial Sequence real-time PCRABI4 gene-specific forward primer
gggcaggaacaaggaggaagtg

SEQ ID NO: 50 - DNA, 23 bp, Artificial Sequence
  Description of Artificial Sequence real-time PCRABI4 gene-specific reverse primer
acggcggtggatgagttattgat

SEQ ID NO: 51 - DNA, 25 bp, Artificial Sequence
  Description of Artificial Sequence real-time PCRABI5 gene-specific forward primer
caataagagagggatagcgaacgag

SEQ ID NO: 52 - DNA, 21 bp, Artificial Sequence
  Description of Artificial Sequence real-time PCRABI5 gene-specific reverse primer
cgtccattgctgtctcctcca

SEQ ID NO: 53 - DNA, 22 bp, Artificial Sequence
  Description of Artificial Sequence real-time PCRCIPK15 gene-specific forward primer
cagagaaggaaaagaagcggtg

SEQ ID NO: 54 - DNA, 23 bp, Artificial Sequence
  Description of Artificial Sequence real-time PCRCIPK15 gene-specific reverse primer
ctcctccttctcctctcccttct

SEQ ID NO: 55 - DNA, 24 bp, Artificial Sequence
  Description of Artificial Sequence real-time PCREM1 gene-specific forward primer
caaagcaactgagcagagaagagc

SEQ ID NO: 56 - DNA, 21 bp, Artificial Sequence
  Description of Artificial Sequence real-time PCREM1 gene-specific reverse primer
cctcccttgctccttccttca

SEQ ID NO: 57 - DNA, 20 bp, Artificial Sequence
  Description of Artificial Sequence real-time PCREM6 gene-specific forward primer
cagcagatgggacgcaaagg

SEQ ID NO: 58 - DNA, 25 bp, Artificial Sequence
  Description of Artificial Sequence real-time PCREM6 gene-specific reverse primer
tattacatccgtgtggggaagtttg

SEQ ID NO: 59 - DNA, 32 bp, Artificial Sequence
  Description of Artificial Sequence forward primer for ABAR/CHLH cDNA fragment corresponding to N-terminal 258 amino acids 52-310
ttagaattcggaaacggcctcttcacacagac

SEQ ID NO: 60 - DNA, 36 bp, Artificial Sequence
  Description of Artificial Sequence reverse primer for ABAR/CHLH cDNA fragment corresponding to N-terminal 258 amino acids 52-310
cgcgtcgactcccttctagtgtcataccagttccag